SEQUENCE LISTING (7 sequences)

SEQ ID NO: 1 - DNA, 97963 bp, Homo sapiens
tggggaggagtatgacacctcactgaccacctgtaaattccccacacacaggagcagaagagatcctcccgggcacctcccacctccacaccagcccaggccctcaattctgctgaccgcgctccccccttgctggagctcgcccatgttggtcacctcacacctggccacctgagttggcagggccctgggcaggtcaccacctgcggaggccaagccgggcctccaggccacaccctcccgaggacatgccagggggctttgggtctgctgctcccagggtcacccagccctggagagcgggccccaggagacactaaccgagtcccgctggacgctcccgtcccagggtctggcacacctgcttgaatctgactcgacaacgtcaactgtaaaacatcctcctgcaacgagtgccacatttccttggaggaagcaagaatctgaaatttaagtaaacatgtaaaacaaagacagagacagtcagctgagaaacccaagacacggtgacaagactgctaccccagggcctggagggtcactgtgagctcggtccccgcttggaacttgggggcgagagtacaggttgacaacgaggctgaggttccacccccgaaaggccccaccattcctgaatcaggctgtgcatacccaagggcctcactgcaaatgctgggacccctgacctcacagatgacaacacagtttcattccccacacaggtcctcagccccagcactcacggggtcagccagagcccccagagccttggagaggggagaggagcctctctcttggagacgtgcagagaaatgatctagggggtgctcagccagggaggatcaggagaccccaactctcccccaaattggcacggagcatctcggcaacccatcaatgaacaggactcaggggcacaggagggctggaaaggaaggcttgctcctttccaggaacagaataacaacaggtaaggcttgaacaaatcaggaagtgccctgccgaaaacgtggcctgctctagccgtggagacccagagggctgaaggagacggggatgacaggggacacactgcctgcaaggtggtgagatgtcccatcaccgtgatgccggaggactgacggcgtctacccttacaactcacagcgtgctgctcctgactgggccccagggcgctggcaagccgcaggaactgcacagacatctccaggaccgaggccatgtcctcccgccggccatcgaactggggcagcagggcccgcagacgctcacagctcaacgacatccgcttcctggttccggtcaagaaacaaatacctctgggccctcctgccctccccgagaagggacagcaactgctagaacctgccagatagaccctgggcgcttgtcaggcagagggggctacaagatgactcagagctgcccccgtggtctgaagccccgagatccacatcagagcacagcctgggcctgacccctggacctcggcgacaggctgtccagcccaggcccaaaccatggctgctttgcggtgtggaaaagacagcgagggtcagaggcagtcagaagcacagagggtcacaggtgcacagcctcaagaggccaaggcttcaggataaagaggggctgcaggatgttccccaacagggcttttctggtttacttggagatgtgcagtctgtccttctcctgggcacaggccacgagcccctcccagctgcctgacacctggtggcagccccgaacataatctcacccgctccacccctttacagcgccctcacccctgggaggcaccaccactcacctgcgctccctctcgctgatcacgttccgccgaaggcaggagctgggaccctcggccaccgtaggggccttgggcgggcccgagccccgggccgagtcctcgcagcaggagagggcaccagacagggagccgctgccgagaaagccaagagcaccgggccctgagaaccccagaaaaggccaccaggagtcccagatgccagaatgggcgtctttggggtgcgggtggaagccaagactgggtcacgtagggacacggcccggccctctctgcaccccttcccctctcccagggtccagggctccgccccaggaggcccaggatgggtggagcccagcaacttgcggaagaacccctgggtacaggccttgggcccccaaccccttggccgccagcccaattcctcacttgcatcccctgacggtagggattctggagacctccgggtagggctcggagcaccgggacgccatgaactcgcagctgcggagcgaccccacgcgcacggccccttccgcaggcagccccgccccctcacgtgtgctctgcccacctgccacgtgctttccacctagcccaccccacccccacttgcaatccgcccccatagcgcatgcgctctagccctccccacgcagccagcccggggcccacgtgacccgcgttcctgcaaagaaggtgctggaaagggactcggggggtccacccactctatgcccccgcaggacgccaggggaaggggccccagcccaggtggccctgcctctacccttaccttcccgggggtcgcctcggttcccacttatgcggtggtctcggtgcccttgggcactttggagatgcgggctctgcccttaggagcccagcccctggtgggagtgggcgggctgggctgagttgtttagcccaggcctgggggacccagggacacagggagggggaagggtacccgcctgctccccagtcgcccacgcctccattttcccccaaatctagtacgcttgcagacgctgcgcctcagggtagggtcaccatccacacttctgggtcgcccagaaccaggggcatcttgtccccaccccaggccaccatcgtccgtgctcttcctcccgctgagctctgccgggcaccctcggaaccgtctgtcccttcacctggccctgcccaagcaccatccccgctttatgccctggggccccagctcgacccctcacctcacactctcaacaggtttctgcgggatcgcgggggtcctgcccatccaaattcagaaagctccggcgccaggtgtttgctcacctccccgctgaactccaggagccgccctgcgctgggcttgagtcccccagtacatacttttggggggatgacggagggaagggggaggtggagacccttcctctttcacggaggttgcggggggccctcagggaagcagaggggacgagggtggggggtgaggctggactgttgggaggacatccctggcctggctcgtgctgaggtcgtagcgggaggaggtggggtgggtggcaggggctctcctcccgagaggggaggcaagaaggagcccgggcaggtgccaccacacggggacgtctgtccggggtgccccagtctcctgtggagaaggcgcggctctgggcctcgggctggcatcctggactttgggccaaagatgggtgaagggtgtttggagcagagggaggttatgtaatataaggtggtgtctccatccctgctccgcccagccttggctgcaggtcagacatggaggaggacggggttggagaggagggcaaaagctctcggggacctgggcccctctcccggccctgcccctggccagggagggatggacgggctggcgatctggggctcctcagtggcagagtggggccaagtttttgtttccctaaagccctcggaatgtcatcaaacctgtgactctcctgccgttgacaacacgtgtaattactgctaattaatcacagatgcagatggtgcggcctctcagttttttctgagttccccaggcaggagggcgttggcagaaaggccaccatcttgccccggcggcacccgcccaggggtggtgggaccccggcagcagcaccggccaatctagggacctgaggaaaaacccccaagatgcccacgagctgtgtggcgggggcacaggctgaacctctgctggggacttagcatcctagacttccccgagtgttccccacccacccctcaaaagcatctgtgttcagcctgctgcctcctggagaaacagggaaggaccctctggagagacactggggaggccagggaccacaaagtacatggcatgcgccctccaaatggacctggccctccccctggccctcgtgggcacagtcctggcttagtgaggggcctggtccaccctgcaaaccaacaaagctctggggacagaaatcgcagttctccagagctctgtgtgaggctgagccagggtgacgcatgtgtaacagtgtgagcacgtgcacgggaaagggggcccgcagggggaccctctgcttcctagtctttggcctgcagcatcttaagttggggcccctccccacccgtctaaaggtgctgtgtgtgcccgtctgggtgagagcacagggcggggtgcagtcatccccctcccccaggccaagcccaggatgtggcgcccacccacagatgccgcagcctgcggggggtgtgggggcgggggggcggggagtcgtcttgaatctggcttgtgttcaatgaccagtgttgaccacggcagagcagcacaagcgggaggtgctgacggtccctgggcgcagcggctggacgctggcggtccgagcagcgcatgtgccctgctgcccgccaaggacccagagccccctgagcccccgcccaccacggagccctcccaggggtgtagggcccggtggcggcgcgcttgggcttttctagccattgacgaaccccgtcctgtttttcggagttttgagggctcagagggccgcggcgggagcggatcgccctccctaccccgcacccccgtcccagcctgggccagagccgccgcgggtcccggcgcggccggcagggggcgcgcgcggacagacgcctgcacccgaccctggaactggcgtcttttcctcgctaatctgcgaggaaaaaaaaaatgtttttcagggcaacgcgagggaagaaggtggcggctcccactcgcttctccctcgggtcgggtccgagctgccaggccgcatgccactccctgacggggcgcggaccccggggggcgtctgccgggaggcgcgcggcgggggctacaccaaccggaccttcgagtttgacgacggccaatgcgcccccaggtacagtctgctgcgccctccccacgcggggaggccccggtctaacctaagacccccaagttccccctcaggctcccgcaccctccaggacccgccattcccagggccatccaacttccccaggacccgcgagtgccctcccaaccaccttggggaacaggggcgcccctcagggtgggccgcactaatcagcaggtgccgaggcagcagcgtccgtccccatctgtcctcgacctggtaagaagctggggggagcggccgtggctacatgggggtcctgagcatctcccaggtgtggggagtggcagggaggctgggggcggtgcagggccttccatctcccccagcccctgccccaccccagcttggggaggaaggttccatccttcttgtgtggcatggcagtgctcggccagggtggggtgcccggcgaggttaccccacacagtagggggtttccagagcaaccttccaaggaagagtccccagtgggaagtgggctaagtggctgtcagatgagggcgggagtcctgggttctggtcactcctcgttccccgctggcctatccctgggaggagagggccagttgagaatggcattcaggtgacctggagctagctgttccctgtgcagtgtcctgggatgccatgcgtctgccgtccacccagtgaccagtgcagaggcacggaccggcagctgagtgggacagtcctgagcggaggttgccccagggtccaggttccccatcctctcccccatccctcagctcttgaaaacaggcaagtctgcccctcaccctgtccccagagccagactccagggaaccctgggcagagccggtcactgccttgggcgaacccctccccaccaggacagagccctttcttctggatcctgcccagtctcaggctgtcctgaggccagagaagggggtggaagagcatacctgagagcgggaggcctggtgcggctgcctgtttgttctatgggactcagtttccccgagtgtaaaatgaggaggtagggcggatggtctttaagggctgcctctggtgagggaccaggctggagaggaggttcccagaatcaagaggcaggcacagcacccacctggcacccgggggagtccggggtacagcgtgtgtgaggagcaggtgctctgagggtggacagtgacttggtgatgtccttgggaagtgggcacagcctggacgtgctctgagggactatccgggggatgcctgtcaggcacagagaaaatgaccccaagggggtcctggattgccctcatctttgtgggctggccggcccctaccatctccctccccagcctagcgttgccctccctggccattctgctggagtttatcacctaccctgctgggcctgacctgctccagacgagccggcaggacagtgtcctcagggtccagcctttagctccaggcctcatcctccgggtccttccagacacctgtctctcccaattggccctgcccaccggcccgggacagctcctcctccccgctggactgagctggcctccgttgcaacagagattccctgtcacactgcaggcagagcagccgccggcagtggagggatgtcaggcgtcaggcttggggagggagcaggacctgggcttggctgggctcgggagcggggagctgagctccaggtgaggccaaggcttgcggcccaggcaggtggcttcagctgcagaaagaagaaccttcgtgctgcctggtggtggggggagctctgtggggcgggtttccccaccagctctcacatggggaggagggtgccaggcaacagcgtggtcagaacccaggccctggagtccataggtcttggtgccaggccttgagcaggcctctgtctttcctgggtgctggaggatgccagccttggtgctatggttcagaggggccaggaggtgcttggacaccccaccccactccagtgccccagctcaaacccccagctccacgctagtggtgtccctgagtcagcctcaaggccatcaggccggaaagtggggctccagagaggagctcctggccttccagtttgcaggggtcttcacgggcctctggcctccaggctccctgctcccacctccatgccatggagtcccaggccctcagctgcctccccacaggccctggagatgacagctgttgtaggacagggagggaccagtggccctggtggccccagagctggcaagtgcccaccccctgcccagcgcacttcgcatcagagcccctgccaggcctttcctggaaataccacttcccagcagctgtgctccagggcgggcctgggaccctctaggccctgatgggatcaggccctggcatcctgccagggtacatgcctggcccaaggtcctggccagccctccaggcccatattagggagcagtgccttcagcagcaggaagtagctcagggctcatgggaaaggtttgagggcccaagataccgcccagcactggccggcaggagggacggctcaggccacagggaactcaggggtctgctgtggcccaggagtggccacagtaacagctcaggaggacagaggtgagtgagaagggagcagacagggtccccacgagggcctctgcttctgttccagtggctggcctgggtgtggcctgtgtcctcgcacccggcagcctccaagtgtgctggtctggttcacccaccgtatggggagctcttgattaacacagaaaattacttgggacgagcctggtgtggcagtagtggggccttgctgtggggccatctcatgccctcggagggtgttggggggcatctcgggctctcccctgaggctttttgtcctgcttccccgggatgcaaacttgcaggctttgacccaggtgccatcggccctggaaagggtggcacttctgtgtctcccacaaacaatcccaggtttctctgcaacaggaaagtttgtccaagtgtgtcccagccctgcatgggtgagggatgctgaacgggtatcgtgtacccaaaaacaagtgctgtgtgtgcatgtgaacctgggcgctccgtgagccaggcaggcgcaccgtgggcacgtgtgtgagagctggaggaggatgtgtggggcccgccagccagcttgtgtgaccttgaagtcaccggtcatgagtctggggaaaggcaggctgggctcagcgtgcccgtgctaggtggctgggtgccaaggcccggttaaatgggcatctttcaaaggtgtcttgggacctctcccagctgtgtgtgtggataaggccacccatgttcacccagacccactttcacctgtggaacttggaagtgagccagcggggctggaactgaggccaggatgcgattggcccagggaggtgagaccagtactagcagctggcaagtgtaacaggtttgcaggggacagtggcagggcagaggccgcgagcttcctggggtaggtccctgggaaggccctccttgagagggggcctccagctgctccctagatcgggggcgggaggtgggggaggtgggagaggagggtggcctcactggagcagaggtgggggcatcaggagatggagggcattgcaggaccagggtctggactggcacaaggcgggcgccccgggaggagcagagggcctggaggagcttctgggaactgtgtctgtgaggttggtccatcccagcactgagatgcagtagctgtgtcagctctggcgagtcacttcacctctctgggcctcagtttcctcatctgtagaatgggagtgctagcatggaccttacgaggttgtgttgagcccatgctgacagcacatacttggtgtttacaaagctgtgttggcgtcgggagcttgcacaccagccagttcgtctttgccttgtcacgcaccagcctgtgttgacacgtcagagctaagcatttgtccggtgacttcccaagatggattcttaaaagtggaattgcccagtcaaaagattcccacagtgttaatgtttgccgcatggcccacggcccccaggggtgagaatggctcctcctcacaacagtgccggggtgtggtgtgtgccagctgccaccgtctcctccttggcaccgtcgccctggggcacctgtgtcctgccaggcaccactcaggcctcggaggctgagacacagctggccccttggtgcccaggaaaggcacacaggcctcctcctttgtagccactctggggcactccacaagaggggcaaagagggcttccatctgcctgtgactccctccaggggccagaagtgggtcctcatgcttcacaggcaccaatagtgcttttgcctggggcagaccccagctgtgacagacagccagggctcagaccaccacacccagcagctttccagtagattttcctgaccccagccacattgagggagggggctggggcaggtattgggagcgccagtgagttggtcattttctgagcatgatggcagctcagggtacgggcccctagatggagcaggggccctgtgcctcagcctccctaaagtggcccagccctcccagagttcattggtgctgaggaaatgaagcctcacttgtgcctcagttcccagacaggcccaaggccaggcacggtgggtcatgcctgtgatctcggtgcttttggaggctaaggcgggaggattgtttgaggccaggagttggagaccaacttaagcaacatagcaagacccctgtctctacaaaaaaaaaaaaaattaaaaattaagatcagataaggcccggtgcacccctgcaggccatgccagccctcaggtgctcagcatggccagcccagggacctggcacagttctctgggggaatgagcaggggcatggtggtgcctcccatagctgtgggagaggggcggcttctcctccaggtgcagcccccaccacggcctccccaccctcaggctgtccaccctaggagggctgtccccaagggtcccctgccggctccaggctgtgctccttgctatgatcaccattgcccccagcagccagctcagctcgagggtactgacagctgacgtgtgcagtcactaagcatctcccagcagccacagcgtggccgggggcaggggctgtgggccgatgcagctggagaacagggtgtggcctgtgagaaggaccagccaagggtcctgggcacccagggcccaggaccctgagatggacttgccatctgaccctgagtgaccagcacactgctgcctgctgcagggggtggggacagcagaagggctcgcctggcgtctggcccaagcccaggcttctgcctcccccgacaaccttgggcacgggtccaggggtgatggcccccacagcctcgcctggggcccctgtgtccactgtgctgctggccttcccctcttcccgccgggctgggagaaagctgctctgaagtctgtctggataaatctacagcttgatttcggctcctccatgtgttctgagctttcactttggatcctaaagcatctcaggagatggtgctgagcagccaccctcgcatgggcaagaagcccctcccctaaagagagacgtcaccgaggacgcttgcctgcccagctgcctgggcatctcgagcagctctgactttttaatcatcgtgcaaaggaagcgttgagttttctgccatggtcagcagagaggttctgcagctttgcacttgctggacactaagcccagcactcagacacccccaccacagtgatgctatgcctgcctgggggactctcctggatgcagggtagggatgcccacacacatacaggcacatgcacacagatccatgtgcatgcacacatatgtccacatgtgcacatgtatacatatgcccatgtacatgtatgcatacatgcacattgtacatacatgcatgcaatcccatccacacatgcatgcacatattcgtacacatgcctctgcatgcaaatacatgctcatttacacacacatcagcatgcatgtgcgtatttacgtgcttacatgtgctcatctccctcctccctaacttctctgtcaaacatggctcgcccagctcctggaaattttgaactgagtgtgaacaagaatgtattttaggagagaaacctcattcccttagccaaagacagtcacaatctttttttgttgttgttgtttcagacagaatctagctctgtcacccaggctggagtgcagtggcgcagtcatggctcaccgcagcctcaacctcatgggcttaaatgatcctcccacttcagcctcctgagtagctggaactacaggcaccatgcctggataacttttgttattttttgtagaggaaggtttttgccatgttgcccaggctggtcaggaactcctgggctcaagtgatctgcctacctcgggctcccagaatgctggaattacaggcgtgaggcaccatgctggaccacaagcaatgttaagagaagtctttgcgggaaaggacattttatccaggacttcctatgctgatcctgagaggcaggctccttgcttctttctgagccccacccggggcagaaataagggcctggtgatgctggagtttagggaaggacatctttagcccttagctgttccgtttgccataacacacatcgtgcatgatgtattcactttgcaaagcacaatgggtgagacgctgccctagaaccccaccctgcccccagcactgtggctgccagtgtgtggggacagtctctatatcacctacaagctgggtgcccagagaaacccacctcttctttagggttctgtggaaagcggtcgtgtttagtttttaaatggggttcattatatgccattctgtttgtgtagtgatgcatccacacgggctcacgcaggctcacagagacctgtgccttcacagagcagtcttggctgtagctgtcagtcgctcctcagttcagggacgtgcaggctgttttcagtgttcaccttcacacatgaggccgtgatgaaaatccatgcctgggcctcctcggccacagatcaaaaatgtccccaggtagaggttccaagttggagaggacccacgattccatcataattaatccactgctcccagtcggctggacactacatgagagccctggcttgcagaaccccccgttctgtaaccacagccgatcactggcgcaggagaccgtccaactctgcatctctctaattattagctaggtggcttgtctttacttgtgcgttgttttccattcctcttctttgaacctcactcatccatcctttgagtcgttttcttttttgagatggagtctcgctctgtcacccaggctggagtgcagtggtgcgatctcagctcactgcaacctccgcctcctgggttccaacaattctcctgcctcagcctcccgagtagctgggattataggtgtgtgccaccacaaccagctaatttttgtatttttagtagagatggggtttcaccatgttagccaggctggtctcaaactcctgacctcaggtgatccacccaccttggcctcccaaagtgctgggatgacaggcttgagccactgcacccggccatgttttctgccagattgttttcctttccacgggacctgtaggcaccaggctgtgttctgatgattattctgttgcaaatatgttcttcctgttggtcgcttattctttccttttgccagtgatgtcttttgttaaacagcagtgttttgttcagatgtagaagcttttataaatctattcctcttggaatagatgatttttgggtttcgtgtattaaataaaaggccttctcagctctgaacacttttccctgtattttctttccatcttgtccttggtttggggctgcagatggggtctaatcttattttatccaacagaagacactgcatagcataggctggagccaggtactgggttcaagcaccggccctcccaaggcaaggggacaagtgataaacaaggacaaataaggaacaaggtcaagtgcatttctgaagttctccggcctccatttctccatcaacacaatgggaaggatgtcgcccctcctgatatttgcaaccgtgacttgagtcaggacaggtggagctgcactcgttccaataccataggacgaggagtgccccccgcctcctttctcaagcctggattccaacgtgggcttcagctggtcaaggcactctgctgtgtctccccactctctgtcttttcttgggccagaccgcagagtgtcagctgctgtggctggacattggtgctggcagcagaagacgagggccctctctgttccttttcacaactgctgtggctgttcttgcacgtatagtcttcaggattaatttttgaaacagttcatcaaattctgagaaaatagtgatgcgctcttgattggcggcgattccctttacccgtgtgtttggagaagggactgttagaccctcaggaagaagacctccgtcttccgccagcgacagcttccagtccatctctcaggaacgctgtgcgcccctgtgggtttggggctcccgcgcctgctgtgagcagggctttctcccggtggctctctgctcctgctctccatggtgctcctgggttggccgccatactaggttccgtatttgctctagtaggttttcagttgattcctcttgggttttccagtaagcaatcatgccaactccaaacagagcccccttcatgactccttgcttttatcctttttctcgcagcagtgcacagatgaggcctcctttatgatggcaagggcagcctcacattcccaactggaacgggaatgcccagagcctttggcgggagaagcttgtctttaagcactttgtcagacagccctggctccatggccaccgggaacagatgcagggggagtgagaagcaggcactcctggctatcagaagagccccccagctggccaaaggcagcacagcccaaggtgaacctggacctggcccccaccccagtgcctctgccgggacctcagtgacgctccttgggctctgggagccacttgtccttgccaggaactagggatggagactggcttctgagaactgttctcaggattccatgccaccacctggcttgccagggtgcctggcgtgcagctggctctcagtccatggcagctggcactgagcacagctgccgggctcccctctgatggggttgtcagggtcagtgcacgtgactacgtgtgcaccaaacagctgacggggacttcctgttgccgtgcccaggtgtggactgcctcgctcattcttggcctctccagatggattccaaaagtcagtgtcccaagcctgagctctctgatgggcctcagctgagcggagggggcacgctgggcgccggccaccctgggtcatagcctttgtccataagagctcagccaaactgctggccacgctacacctgctgtagctgaagcactcacctgcaggccctgttccgcgtgcctcaggcatgctgtctggttacatcttcacactcttcctataagggaacaggcatcagtatctcccttcacgcatgagcatacaggctcagggaggtgaagtgactcgccgggggccgcccagcaggactgatgggccaggactgaactcatgccagcgggactcttgccactctctgggcctgcagggtgtgtgctgacagcccagagagtccccaagaggagcagcggggtgctctgagcccctgggctgccccggccttgtctagggggtgtgctgtgtcccgtcacctcagccctggctccaaggggtgtgtgagtcccacaggttgagctccacaccttgtccagagagtagtgtaagggctgaggagagggacaccttccttaagccaccaaacacagaattcttatggaaattggtaattatttggcacagcagtcactcagaaaataaaaagatcatggatgtggccttgggggtcctagatttcctccagggccccttgggtcacacagggaggacagcgcaagagaagagggggtgcagaattcgaggtgggccccagggtccccgtgtctggctatgccagggttatgttccagttccacctgaaccagcttcaacagggaccagaccccccaacactcccctctccccctgtgcccaaaaaagctggccgatgtaggccttcctgcttgcgaaaagtctcaaccagaccccgaggacacggggagcagctgttttgctgctgtcctcctgcgatgggcccctttcccagagaccctgtagccccggccctcagtcccagccctcagccaacacaacagccaaggattttagccccggccctcagccaacacaacagcgaagtattttcaaggtttctggctctgtggggggaacaaggcaggatgccaggaggcctgcaggacacctgcttatggccgtgtgctcgctcaagtgtcaaagcagcttcccaagggaagaagggcatcttgtccttgtcaccctccccaggccacatctggggagcaggggtggggccaggtctctggactcctggtgcacttccttcccctggtgactcctgaagcaagccaaggtccaagcccctgagcttgtccatgggccctgccaagactgggccttgaggccaggaggaggaaccagagccgctcaaaaggcagacggggtggggacgggaggaggcagccacgcctgaggagccggcagtctcttggggttttgccattgaaaggggctttcagtgaggaaggcacaaaatcaagaaaatagagccccgacccctggccacgtggttcccctgcctgcctgcacctccccatggcccagccagcttgtgactaaggaaaaacatgcagctacgctgatgcagttaggaaaatgttattttttaaatgctgccagatcctgggggttgaacctgtgacctcagaacaaggagcagggtttggggcacgcacctgctgcctgcagcttcagcctggggtgcccatagctccttgcctgctgcttgtctgagggttaggggacacaggagaggcacttggtgggggctcaaggaatgtgcccacccctcccgacttgaggagagagaggagagttttgtagcatccctggacctggtgttccaaatgagtggcctctgtgcagggcctacgggcacagcacggggagctgggtatggtcagagcctcctgcccctgcccgtgggggagccagccccctccatcccccctctgctctcctgctggcctcgaagccttgaacaagggccggcctgggccaggtaaggagctccctgcccctcagctcctgcttctcctggagcagccaaaactcacttcagtgggggacatggggacaggaaagatgcaagacccctggaagacacctgcttgggcccccaagaactgcagaccaccaggggtcctcccactctacctggtgtgccacgatgcccctaacctgcgctgccagctctctgtgtccctttgatccctcacctggtgacagtggcagggcagctcagggcccagagggcctggcagccccccggggttctgacctgagctgagctgctccgcctggggaggctgcaaacaagggtgtcttcttaggcctcctggtcactccaccctcacccctcaacagcctgagggtctgagacgggggtctctggggctggaccctgaaggaagattgagcagccggcctcggcaaagaccctggggacggggaggggagctggggagttgggaggggttgcaggggtgtggcggggcttcagagcagggggagggcctccatcctggctcttgggggatcagggaggagtggtgcgcgcctgtccctgagctggccatgacagctcccgtcccccttacctcctgccaggccccggggccgctttggagggagccccgccccctgcccctgcgcagcccccgccctagccccagcccggcgcagccggtcccgcgcgcccccgcccgcatgtgccgccaggcccgcccccgcccggccgcccgcccgcccggtgggtcgcggtggccgcgggctgcgctgcgcgggccgggcctggcgggccgggggctgcgcgcgtccgcgagggcgcccgacgcgggctgaggggcgctggcgtgtgcccgcaggcggccctgcgcgggggacggcgcgctcctggacaccgccggcttcaagatgagcgacctggactccgaggtgctgcccttgccgccgcgctaccgcttccgggacctgctgctgggcgacccgtccttccagaacgacgacaggtagggaccgggcgcggggtgggggctggggtcgccgtcccggcgccgccgcacgcccggagctgtccgcggtgctgacggccggtcccgcgcgcccccgcagccgccggcgcccttcaacttttcccggcttctggggacgcagaagtttcggagggggtgcgggcagggggaagcatcttctcgggagggggtctccggaggagggcgcgggatgctcggagctgcggagtcttccagagcccgacttggggcgcggaggcggagggcggcctggccttccggtgtctgctcccaacccccagcaccccagtttttctggttttgggggcaagccagggccggaagcgccccggtggcggagcccgcagcctggctgagggctggcgtcctccaggctcaggcagcggtccttgattaattattcaatgagggagcttgtccttcaggttcgtgacctggtcacttattccgagtcaccgtgtccccgtcccctcaacatctcctgggctaaaaaggcaaaatgaattgtgtctttcagttaaggaccaacaggatgagcccatgggaataattcatccgctgagggggctggcaggaagctggaagaaatgttgcttttctctcctccttttccccaaatagctgtcttgggtaaacacccagcactttagctctgccaggctctggggaggcgcttttttgcctgcaagtgaaggcaagtgctctgaggactcacgcggagctggagcccgcgggggcttcaggtcaggaaccgctcctcttccagcgtgtagggtacggcagggtcggggttgtcactgtctccagggctccgtcaccacccagccctgggctcagctagcaaatccgcactttcccccaacaacgcagttattccggttagcacacaaagtacacgaggttgaaaatggtcaggacacaaacagctttcagaataatctactgctgaaaccgcagctgaaaaagaaatcatgtttgtaactagagtcagtcctagggactgtatccatgaggaccatgaagatttgtaaaaagggggtcttaaaacccctctgcctgcggggaatggaatattctaccagacctcccataacatttggttgtgtgaaccaagagtcaggagtggggactgagcccagtcttcatccactcagaatcagggagtgagcgtcctgcagagaggccttttaaacttggtttttcatcttgttgaactcagtgctctaataatgagcccacctaagtgcaaatgcccaggtgaagagtttagtttatctccctgtgtgggcagagggcagtcagccaggctggtcccagggcggcgtgcgtggctgggaggctctcctgggagccacacaaagcttccctaggccgaatccgtggactcaacatctttctaagccagcaccagcccctcagatgcctgttcagctgggacttggttaattcttgaaaacggcagccctgggagatccttccgatattgggcaggaagtggtgtccatccgcctaatggtgcctggggctcccagatgactcgataacgcctgtggctgtggttgatcaaattgtaatgctgtattcctgagtgttcaggagtggacagcagaggcttttgctgtccgcctagagagaaccatgcccatccctgggagaggagaagctggcatgggatttcacaatgagtccctgatcctgggtgtggttttttaaaattgtggtaaaatacataaaacagaatttaccgttagtgacatttagtacacaacactgtgccaccctcaccactgtaattcgagaacatttgtataccctagaaggaatctccttaaccactgggactcactccccaatgcccccttatcccccagcccctggcagctaccgatttgcattctagcggtcttggcgtgctgtgcccactgggcacatgggttgtgtgccgatggtattaggaggtggaccgctggacgtgtgtggctgtgtgtgctagctagtgcgtggctgtgggtcctctccaggaaaggcccatggggtacagggcatctgtgccacccccacaagtgccctgctctctctggttggaaatgaagtatacagtgaggttctattggagggagagtggccagggattagagcttcggggctgtgtggtcgccatacctgtcctgtggcgctgtccctcaagatggcctcagagatgctgtgctgcataccttttctcatgtgtgaaggatgggagtggacctggccctgtggtggtggtgtgcataggacgggaggggacctggcccctgtggtgttggtgtctataggacaggaggggacctggcccctgtggtattggtgtctataggacgggagggaggggacctggccctgtggtgttggtgtgtataggacaggagggaggggacctggccctgtggtgttggtgtgtataggatgggagggaggggacctggccccagtggtgttggtgtctataggatgagagggaaggggacctggctcttgtggtgttggtgtctataggatgagagggaaggggacctggctcctgtggtgttggtgtctataggatgggacagggaggggacttagcttccctctgagagtggttccccacttctcacctctgagaggaagcttgggcctggtggagatggtggccttgggcaccccgagatctggaaggtggggggctgctggtcggctggtgtccagcccggttcccagcagctttgcctggctccccctggaggctgcgggggctggtgaggctggctgggcacagtacaggagtgggcaactttccgggggcgcaggggcaagtgagttgggggcttctgcccaagtcacctgccagctcgccagccgctgcccggcacagctgaacccgctcctcacgtggactcaggccctcctgagaaattgcctcgactggatgtaagagagggtgccctagaggcctaggagcccggggaacccccacttaccccacccaggaatggcagctccttggtctcagcagcagcctgggggcctctcctcttgaaggcagctcagggagcctggcctgaagggtttctcttcctcaaaccttctttgcccaaaggggcttgcttcagcctccatggtagggttgggtgagtctggcatggcctctctcatgaccccgatgctggaatgggtgggcaacaatgagggaaatcaggatgctgacaggggtcctgaggtcacccaagccctcccctatccataaggacacgcacgtggggaccccagccctggctctgtggaaaccccaagctgacctggaggaggggatcagccgcttggaggaccctcctgggtgcttggggtggggtctgatgcctctatctgccattagaagccatcccatatctcctcatcttcctcctcatccccagtgcctgtgccttccccttcctggagtggagaccaggggtcgtctgcctggcctggtgggacccccttcctgtgtactgcagtgactcacaggagccgtgtgaggtgggaggacgtgcttatctgaatgcaggagggagtgcaggcggggtctggaggccatagggggcccaggtctgaaagggctgagcccagcaagacctgcctgccggctctgggtgagaaagcccttaggtagaggggaggggacactgggatggggtcctgggctgtggacggaagggctggtgtgcccggacacggcagcacaggctccactcgggcccctgagggaccatcgtggtggccttggccatgggggtgtctcaggtctgggtaaggtggaagtgccgcttggtctctctctgcaccaggggaagagattgaggggcagggagacccaaccatcagcacagggcaggtgcaatgtgggggctcagcacagactggcacccctcacgccctccctatttctaccctggggcaggctctgtgagggaagcgggtgctgctgccaacccctggcaggctgtgagcgagcaggtgggcgggcaggactcgggcttcctgtttcccccgggagtagagcctctggcagtgagtcaccacagcctccatcgcccgtggccttggacagagaagagctcggcgggccatgtgggcagaggaggctgtactgctgggcaggccgctcccagcggggatgacccagcccatcggtaccgcccggtggaggaagtggacagagttgggagctcactcacctcagggacctgatggtggaccctcaccactgcttaccagatggtgtctcgggtgctcccatggagcagcattcagccacagtccacctgcgcccaggctctggagccccttgagccatgctgacagtctcactgcccctctgcctgggagtccaggggcaactgatggaggccctggccctcccagccagagccagctctcaagagtgtcccggaagcctaagtgagcgtcatggcccttggaggacacagacttgggctcttcttctgcagaggggttaacagcccccaaagggcagctcgaggagccagcgggacaggggctgcctgagggcttgggtcctgacggtgtccccgagctgggtcccaggcaccttgtgcagactctggtctgtggcagtggaggtgcggcttccatcctgccctgcactgaggctcaggaagatgctgtgtatgtcgggggatgcatgctttggggacacttggggctgtcacaccacaggcccatctctccaaaggggcagattccagacaaggagtcccagtggggaggggtggggcaggcagccaccccccgccatgctgcaccaggacccagcccaccggggccactgaagccaccactggaggcaacgtcagggtcgccaacactcccaggcattaagcaggacatgagaggtgccaggaaggccctgcccactggctggggcttccggcaggcaagaggaagcttcgtccctgagcatggccagggccactgccttggctccttgggccaccccaagctgagtgccccaagctggtgccatggggcccaggcaccagggtcacctgggaagggagtggccgccttgccacagaaagcccggccccagtgcaggcctcccggggtcagaacactgccccagagcggcctgaccaggctgtttcattagctcctctgacaacagagaacggaacttgacttggttctctggagttgagcaacaccaaatattttttcacacaacaagctgagaagtggaccacaaacagggcccggcattgctgggaacacggggcagcgggcctgcgtggaggatggacccctgccggccccacaggcagcaactgtggatgggccaggcagggccagcaggagcaggaatgggtgcagaaggagggggcagcccagacccctccctggccctgctggatgcaccccttccattcacccgaccctaccccacagtgaatccagctgccccgcagggacagggtagctgggaacagagagaggcttctctcacagggtgaaggccccaccaggcctccaagcagaaggggttgcggtggggatgtctctaggaacagccctgccctccgagccctcatctgtcatccttaatagctgaacaatttctcagctggtcggggaagtgccttgagacctgggtgactcggtgtgctgagggccttctagaatcatccctcctgcgaagagcctgggattgcataagtgccctcctcctgcccagccctcccagctccatcatctcttcctatcccctccccacccccatcatcctgcccccacacaccagcctggcccctgctgaagaagcagggggcccaggcagccttcctccagagggagctttgctccgcctctcttggggctgggggcgggggtcctgcctgtacccccaaccccacctgctctctgaaggccactctgggaccctgagctgagaaggcccgggagccagccccagctccctgaagggcactgagcccagagtttctgcgtgaaggcgtgagtggcctgggcaggacctagggcacaggtcagctctccttcgggggtgctctggggaagctagggctgggctgcgcctgcaccaccctgctcctgccttagggccccaggtccccatgtgacagactcaggtgtagctggagctttgaaacagagggggacagtcaggaggaagggtgtggccaggcctgacccaaaggtgagggtggggcacgagggacacacggccgagcccaaggggcagtggtggtttaggggccgtgaaggttcgaggaggagtcggggaaccatgcagggtgagggctggggtgggagaggcctggagctgagtttgggaagggtggcccctggactgggggacccccagaaggggctgaagtggcccccaacgctctcggcctgagctgctgggccagccccagcctcgaagccactccgctccagcaccgggtcccctggcgtgcctcctgcccagagctgctgagctcagggaaacgccagcctgctcagtccctgggggtggtggggtagggggtggttatatttaacattctgcccggggccatcctgctgtgtgcaggcgtactaatagggcagggaggggccaggtgggatcccagtcctgcccctggcccttctcgccctggggccctttccagagcaaacacccacaggcttgggaggacacggccagcctgagaaatggccttggccaggccggtggggcccaggcaggctggggtctcctggccgggggagggtgccggccatgcgcataccaggagccaatcccagagcctcggggccgcctgtctgtctttcctggacccccttggtgtgccacctgcatgattgagtcaaggtctggtttattggagcaggaaaggcccgggatggtgtccaaaagttctggcaggttccatgtgctttacggtgtcctgagtgagtgaaatgcatgtcatgatttctccagtccctttccagctcagggtcggtttgcctgtgttgcccccactcgactgtggacagggctggaggccaggcctgctcgcccttgtctcctgggccccctaccaggaagcagggtcctggaaggtgggcctgagtcagggtgggggaaggtgacgcctctgctgtggagactgtggctcccgggtgggctgcaccttcctcctagccctgtccctccctccgacggtgggttccaggggcttgcgttgcctccagccctgggtgaccaaggagtgggcaggatcaacaggagggccacagaggctgtcggtgaccagggcccatcacatgtgaacggtgtcttgtggggcatcacctggctgtgggggcatggaggagggtccgtgatacccctgggcccagggctggctcagtcctcagcctgtgcgtctttatgactcaagcggggggcctctggctcccccaaacacacccccaggatatccagggagaccctgagagggcgaatcccgcgtcccctggggctgcatcccccgtcccacccctggggccggcacagcggggcacacggcttcatcaccaagcccatgtcccagggactcatgtgtgtgacacaaaatgaaattcctcccgcattagtgtaaaacagaacacagatgcccagtgggaggggctggcatcctccgcgtcaggaagccaccaaccgtttgggcttcaggttgtaattcccaatgcgctccctccactgttccgtttccatcatccggcgcgtccagctgaaatggaatgggtgtaagtgtgtttatgcctcgtctcagtcttcccagctgctatggaaaagcaccacagacccggcagcctcagcaacagagatttatttctcagttctggaggctggaagcccaggatcaaggcgccggcggaccaggctcctggtgagagcatctctcaggcttgcacgtggcgtcttctcactgcgcccacggggcagagaatgcaggctctctgctctcttcctgtggggcactgctcccatcccgaggccccactctcatgaactcgtccaagcctactcacctcctgaaggccctgtctctaaataccatcccatcagcggtgggggcttcaacaagggaatctgggggtgcaattcagcctggagcaggcttggagggaaagaaggggacgctgcaaaagatgtcgtgggaaatcctccgggggattaagttgccccttcatttcaaattccttaaacttcaagggcatctgccaggtccctcgggagagcctcctgcatgcagctttcctctcctctccagcgctcatcacaacagcggccactgtgtgagtgaaggctgaggccacactgcctgtgcctccccagcagtcagtgacgggcgtggctctccgaggcagtgaacttgcagagtgcaggaagctgggggctgcagagctcgcctgcagggcagcaccccagaggcctggcagggtgggtgagctgtcccgtgacccgtcacccctccaccaggtcccaaaagcaaaagcgttggtggggtgtcaggagttccagctgtacctagaaattgtcctgcatcctgttccaggagatatcgtccatcttggggtcaacatggaatgccacctgcatgaacagggccggcgccggcatccactgccattgcccgaggcttggctccacagggtggcaggcagggaggagaggctgctgctccctggcaggaagttcttgtgcaccacgtgagcgtgtggctcccgaaggggcattttgtgcctgtgcgaccgacagctgtgcccacgcgtctgctgcatggccatgtctacacggggcctgctgcctccagagggaccctggggcttggggggcctcagagccctgtcagtaggaagggggccctggctctcctggtgggtgtccggagcatctagagctgtcccgggaagtgccgtgtcccagggaccttgttactgatgattctgccagtctggcaagtccatcaaaggcatcaccgccttcagtgccaggccacccctcggggcttgcaggcctggtgtccggctgcccccagcccagcacaggtacctcatgtccttgaacaccccctcctccatccagtctgagcgccatggctcagcgcagccccgtcccagaccagtgtccgttcccctgagggtgctgtgggctggggaggcccaggtatccctgccaccttcactctgagcccgtctcttcctctccaggttttcataagaggccccgggaagctccatgccctcctcccagcactcctgctttcccctctgaatttttatccatcatgggcccctgaataagagctgactgtgggatccaggctcagaggctcgagaggcacagggggagggcaccaagggccttgcagtccaggccagcctcagggtcaggcctccaggggcctcaactttgtgtcctccccatggggtgcttttcatccccggaggccaggctgggggtggctcagggggcaccatctgcacaggtgggggcagccgtgggcgggtagagacagcgggccccgcgtgcactcccatcctgagccccctgagcctcaaacaagagtgaaggcagtgcccagggaagcacaggccaacgggggcaggcacagccttgtggtggccagagaaccaggtcctcacctgcaggatccaggcaggccagtggtcggcgaggggcgtggccagtctgcctgttccttcctccacgtatgcaggcattcattcattcagtcatgcaacagaggtcaagttgagtgtctgctgtaggcaccagattcagcaaatacaaatacaggatgcctggttaaatatgactgtcagataaacagtgaatcatttcccagtggaagtgtatcctatccaatatcggggacgtgcttatacttaagacaattattttccatctgacattgaaacctcaccaggtgtgtcatgtatttcattgggatccctagctccggctgggtcggctgggctgccccgagaggcccaacatgaaggccccggccacccacctggattggagcttcctccgcccaggcagagcctgggctcctgcaggcacagatgggccccccaccactggcttcgaggatgaccctgaaccttccctctagcagacaacagatctgacagggtcttccccccatgagagggggtgcccctgggctcccccatgtgatcctgagggtaatcttggtggaagcagcgctggggcctgtccctgccccacccaggaggcaactgcgtgggggggcgtgtggtggacacatgacaaacccagcccaggatcccttggtggctcaatggcccaaccgcctccctgcacagacaggaaaacggagtcctcagaagggcggggcctgtcgagggccacacagctgccgggggggacctcagaaggaggggtctgtggagagtactccaggtcccctatagtgtttgaacagggccttcgaaagattcctgtcctcccagaaccccagaatgtggcttatgtagaaacacggtctttgcagatgtgattagttgaggatctcaaggtgaggccattctggaccgaggggctcagatccagtgacttatacgaagagaaggaacaaacccagagacacagagaagaagccacgccgggacagacgcagagatcagagtgacgctaccacgagcccagggccaccaggagctggagacggcggaggacccgctacgcttgcgggggagggtggacctgccaacaccaggattttgcattctggtctccagaacagagagaagagaccgctgtagttttgggcactcagtttgtggtattgagccccaggacacccatgcacaaatgagagggtgcaggggtcactctgggacagcaccaaagccgggggggccccaggggccactaaggaggttcccacgtggagctgtggctgcccttgctgggtttgggaccctcagccgggtccaggcggacagagtctgtttccagcacagccccgtccttttgctgggcggtttgcctgggcgtgtcctaagttgattgaatatatgctggaaagcagaaatgccggaaaagaggccccctcccacccgccctgggagcccaggtctggcaggaggaaccacctgggcgataggggtctggggaggaagaggggttggtgggcagcagcctccccggccaccacccaggagccatggctgtgggggctcggagtggccaccccacccaggaggaggggcctgtcagggcaagagacccccacatcctcccctgaccttgatccacagaagggccggcaaaggactggggagaggcctgcagtcgtgggggcagggggatgaggggaggggacaggaggagccagggcgctgtctgggctgggggctgggctggggcaggggcagctgagggcctgcggtggatgctcccagcccctctccacagcactacgtggcacagggccagcagcccccgggggagactcccggcctgcagggccctctggggcctccctgggtctcggatcccttccagggacctggatgaggccgtacccccagggacagaagtgcccctgggagcaaagccagcccagtcttgggtggcctgagctcagagctccccctggcttctgtggctcgccatgtggccagggccgcatctttacccccaccccgttccaagcctcagtgggtcccttggtaggtagaacctgttgggagggtaaagcgaggcaccccaggtaagccaagttctgctacggacggggccccttggagacccagatcagactctgccaaggcctcgccctactcccgcccccaacctgatgattagctaattaattctgtctctccctccctctatctctccttccctctctctctctctacctctctccctccctctctctctctcactctctacctttccctccctctctccctccctctctccctctctctctcttcctctctctccgtctttcctattctctctctctctccctctctttcccattctctcactctctccctgtctctctccctccctctccctctctctttcccattctctctctccctctctccctctttcccattctctctctctccctctctctctctccctccctctccctctctttcccattctccctctccctctctttcccattctctctctccctctctccctctctctttcccattctccctctccctctctttcccattctctctctctctccctctctccctctgtttcccattctctctctccctctctctccctctttccccttctctcccctctccctccctccctctctccctccctcttcctctctccccccctccctcccccctttctccctctctctctctttcccattctctctctccctctccctctctctctctctttcccattctctctccccctctctctctccctctttctccctctctttcccattctctctctctccctgtccctctctttcccattctctctctccccctctctccctccctctttctccctctttcccattctctctctctctccctctccctctctctccctctctctcccactctctccctctcttttcccttctctcccccctctccctctctctctctctctctttctctctctccctctctctctgtcagtttccccactctctgcagggtcccctccacagtgattagtcaaggcccagcccgaggggcctgggaacggggagggggagcccagccgtgtgtcacctttgggagaggaggacctcttccatgcctggtggtactgttgtgcctttgaagggaggtgtggcaccaggccgggtggtgtccgccatgccaaaggccactctgcatcccggcccccagcccctgggtactgctcctccagcccccactcgctggcctggattctccggacagaggtggggcccacaggggagcaaaggaccatccagagagcgtgggtctgtgccagggcccttctcagagcagccgaaggatgaccatcctgactgaccgtcccaatctgcagcctgggactgggacctgctgtgcagggctctaaaaaggcgagtgagggaaagatgttgcagtggggaggccatcctggcccaaaacgcagtcacaaagtccttaaaagagggcggcaaagagggttaaaatggcaatatttgtgttatgcacatttactacaaaaacaagcttgccttaagaaaactcaaaaaagaaaaagattaacaggcagaaggagatggccgtgtgaaggcgggggcagagactggagtgatgtggccaccgccgaggggtgcctggagtccccagaagttggaagaggcaggaaggaccctccctagggcctccaaggaagcgtagccttgtgacactggacttcagcccagggacactgatgtccaactctggcctccagaactgttgcttcaagccccccgctgtggtactttgttatggcagccccaggacaccccgacaccaggaaatcgcataattgtttggagggagaacatcggggcagattcacctggtttctatgtggggcagaaaagagccagcggccaagccacccgcaggcccctccttgggaagtgagacccccacagcctcactgcggatgtaacagccacccacggcctgcctgggaaaagcccagatttcaggaggccctgattaggtctcccaagactcacgccctgttccgggggaacaggaagtgggaggcatctccccagaactcagggaggccaaggccatgcccacggaggggcagagagccgggggccggagagtgagggccgcagccacgggctgggaaggggagagggccgggcaggggtcctgggagaggggcagcccaagcccagcttggagagacatctccccagcggccccggcatgtgctcagcagctggtgagctcaatccaggagtgttttgccgggtctggttatggtgattcacaaacccctttttaatctggaacagctcccaccaggcgggtcccccaggatttcccaccctacccccgggattgtgaatctgagacatcacaccacgattgtcacgtgacatggtgttcgggattgaactgtgtcccccaagatttatatatcaaagccccaacccatggtaccttagactatgactgtatttggaaattggttctttagaggtaattaaggtgaaatgaggtcattcagttgggccctaattcaataggactaaggcccctctttatacaaaggaggaatggggagacaggtatacacgggagatataattaagttcgctgagcagttgaatttgaattaatcaaagggcgattatccgagtgggctgcgtctactcacacgtgccctctgaaagcagcgtttcctctggctggcagtggaagaggtgaagtgactggaggcagcagggaactgaacacatctgtacttgctttagagatgagggcatatgccaaggaaggaggtggccctggggagctgagagcaaccccctgccagcggccagcaagaaagggggtcctctgtcccactgtgcaggaggggattccgcagatggcgggaatgagcctgggagcagattcctgctgggctccagatgcgggcccagccctgcggatgcctccgtctcagcctcatgggaccctctgcagaaaaccaagctcagctgtgcgcagacttctaccctacagaactgagaagtgataagtgggtgctgctgtgagcttccaggctggtggtggttcgttacgcaacagaggtagcacgtacaccaggcctctcgtcctgcagaagtttccaccgtctgcacgtgtccggtggcctctctgtgggtggttcagcttgttcttctgcctccagtattttttgcaaagtggaagttgggtctgcaggcctgctggacagagcttacttgatcgaggcttgaaaatgttgcacgtgcccttggtgtctgcgtgtggcctcaggtcctggttgcccgtgtcaaagatggcctgatggacctgctggggccagcccaaccccaccactgggcaggtacttccccctggtcacctcaaggtggcctgtggctttacgagcacccagtgttccctctgtggctcttggccccctctgaaggcccttcccaagcctgtccttccactgggacctgggaggtgcaggctgcctttttctcttcctccctccatcagcgttacccacattctcctctgcactgtgctgatccttcatccatgggagctgtgcacccccaaatataattcctgctggaaacaccctcgaccctttctctttaatgacagattttcacagtaaggcagtgttgaattgcagcctctagtggcgacaacaaggaaaggggttccctggactcccccttcggagcatcaggtgtggacccatggagttccgtggacctgccgtgtgtggtgggccaaataatgctcccctgaagatgtccatgtcctgatcccagaaacccatgactatgtaaccatgcgtggcgagggggaccacacgtggtgagggggattagcagatatgattaagtggagggccttgaagatgggaaattatcgtggatgatctgggtgagccaagatcatcacagattcctaggggagggaggcagaggttcagagtcgctggagaaggagatgagcaggggaagcaaggactggagcgatgggaggaggggcccagccggggagggtggtggtcaccggaagctggaagaggcaaggaaatggacccctccctgggactccagaaggagccagccctgccggcacttcaggcttgggatttcggacctccgggatggtgtgcttaaaacacttgtgttggccgggtacggtggctcacacctgaaatctcagcactttgggaggccaaggcaggcagatctctcgagctcaggagttccagaccagcctgggcaacatggcaaaaccctgtctctacaaaaaatacaaaacttagccgagcatggtggtgcatgcctgtagtcccagctacttgggaagctgaggcaggaggatcacttgagcccaggaggtccaggctgcagtgagcgatgttcatgccactgcactccagcctgggcaacaaagtgagatcccgtctcaaggtaaaaaaaaaaaaaaaaaaaaaaagtgtggtttagcaattgaatttgtggtcatttgtaaccacagcagcaggaagcccacacgccatgtttgaatcattgtcccctctgagatcccaccgcctccactttggccaatgggagcccttcccactggcttccgtacccttggacatgaccccattcctctttgcaacttccttgttctctagcaaaactagattttccacactctcgaaccttccctgccctgagtcagtcattctctggggatgggtctttcagcgcggctcgcgttagaatctcagtcgcctggcgtgtcttcattttggatcacagtgtcctgcatacccacaatgttactaattttatcattgtttttactccaccaaggtttaaatgcttacctgttctataaccatattttgtgtaattctctaacgctctgtagccacctcagtgggcttcccgctgcccacccaccctttcatacggcttcttcacgctggaattctttctttggcatttcttttagcagcttcatttcatctttgaggtggctgtggttgtttttctcccaacaagggcgtgtgcatttgtgccgcgttctctcacgttcctttgcgttggaggacatcgcccctgtgtctctagactcgagtgacattttggctgggtataatagtcctagacctcattttttcccattagaacttgatcagcatgggaccgtcatcctctggtgcttagttcactgcagaaaatctgcagccagctcacttcacccccttcgtagtgactcactggttctgtcttgtgcacctgcagaatcttctttaccctcagctgaggaggtggagtaggagccatatctggatggaatgcatcctgtgtcttgttttcctgggactccgtgcacagttctttctttgcttcatcatcacatatctaaatacttttcaaatatgcgtgtatatatatatatatatatatatatatatatagagagagagagagagagagagagagagagagagagagagagagagagagggagtctcactctgtcatccaggctggagtgcagtggcatgatatttcagctcactgcaacctccgcctcccaggttcgagattctcctgcctcagcctccccagtagctgggattacaggtgcccaccaccacgtccagctaatttttgtatttttagtagagatggggtttcaccttgttggttaggctggtgttgaactcctgacctcaggtgatctgcccgcctcagcctcccaaagtgctgggattacaggcatgagccaccgcaactggcttcaaatatattttaaataccaaaaactagttgaggcttctacccaggggtacaagcaatcctccagtggaagtgtcctcacccttcctccggtgcctggatcttgttgtgcctttgctgtgctggtcagagccagccatctgtgtctataatccaatttcagccatgcctctcctgttcctcctggcttgggctcattgagtcataactgtatggcgtgtgggctctccgcttgcttctggagttctgtggtctccccctctgccactggctgctgagatgaacaggctcatctcatcttcgagtgtggcttccattgcaccttgtccttatcaagttgacacagtgcccaaattcctcctggggaacttccttcttctctgggttttgttttattgttggccgggttctttgtctgctgaagtccaacctatctctctctctctctctctctctctcattctctctctcttctcttttttctcccttccttcctttccttcttccttccctccttcctggggctccatgcccttctcccactgggggcaatcaagaggtctcacctcagtccaagggtcagccctggtgtctccccagccagacagtgaccaaagtcaggacacaccccctggttgcctcctgatgagggctaagggtgaggattccggcactcccagtttggactcagaagacaggagcagtgtggggagcagcaccgcctgtacctcctctgtgactcccctggacaccctccaggaggtctgtgatgctgacctgccctctccttgtcggctctgagtcattcatctccagttcttactatgcttgtttgttgtcctgggttagtgcagccctcatacctgcccccgcacctgcccccacacctgccttcacacctgcccccacaactgccccccatacctgcccctgcacctgccccacaactgccctcacacctgccccccacacctgcccctgcacctccccccacacctgccagcacacctgcccccacacctgccctcacacctgcccccacacctgccttcatacctgcccccacaactgccccccatacctgcccctgcacctgccccaactgccctcacacctgcccccacacccgcccccacacccgcccccacacctgcccccacacccgccctcacacccgcccccacacctgcctcacacctgcccccacaactgccccccatacctgcccctgcacctgcccccacacctgccctcacacctgcccccacacctgcctttgcacctgcccccacacctgccctcacatctgtaaatgtcacactgcccagccctgcactcctctcccacttggccagtttcaggctgtaccactccaccgaggtgaccccagctagtttggggcttcagatcccactgctgagccggagacccagatgggtctcagactgacagtctcagactggcagccccccatagcaccaggcgtgggtgcccacccacgagatgtcacctggcaggcactttgagaatgtccagaatgggtgtcataatccaccccccgcctgctccttttgtcttccccaccttggggtgtcaccacctgcctgttccctccccccagcctgggcgctgtccctcccccctagtccttcaggaagtctagccagtgccaccttcaaaacacaccttggatccacctggtcccctgtcctctgtccagcccgcagtcccactcagctcgcggtaggcaaagcgaggccccaccctctctgcagggctcttggagacacgtttgaaccacagcgtctgtgtgctctctaaaagggcagttgttattttttcctgagtttccaggatgtctgcccctgtgtagccatagggtcgagcctctccaggctccatgggagcagaaagtgctggggacacctcaaggctcacgtggctctgtggaccccgagcctggggccccagcccaccttcccctccgtgcctggctccagccatgggccctgcacccgtcctggtgccctgaggccaggaaagggggcttctgggactgctgctcgatgccgaaggtattgtccttcccacatgggaatcatctcactggactttctgtgacaaacatagacagtcatttcaaaataaatcaaacttcagacgtggtacagcagcaagttgcgaagatcctcctcccctccccgcaccccaggcggcacctgctgcagccggctgtgggcactccatggacaacacggcacagatgttctcttgtcacataactctcatcctgtacttcaaaggccacgagtaagtgaagacccactgcactctcgaaaagcaggaacggaaaaagtcacagcactgcatcatggagcagccgccccgggtgggcgcgcgcgtttgcctcctgcgtgaggcttcctccagcactgcggtgggctgtgggttcgttcccagggaatgtgagcgagccgcatccgcagcccaggcaccctccctcccagcttcctccccgccccagggtgaccatgtatgggcagccctgatgtccatcggccctttgtgcagtcatggcgcggtgaagggccctgcaggcggcgcccaggggtggtgcggggctccgcctggtctgtttactcacagcacagcatggggctcactcagcagcacgcccaggagaagagcccagcctcattctttacagccgttctggggcactccacaggagggttgaagggggcttccatctgctggtgctccccaacaccaggggctggcagagggtactcgtgctttgcaggtacaaacagcgctctcccctggggtagacccgagccacgacaggcagccagggcgacatgcataggagcgtgtgctcagaccacacacagtggcttttcaggggattttccttactcagccacatcggggagggcgctagggcaggtatcgggagcgccagtgagttggtcattttctgagcatgatggcggctcagcgtttggacccccagagggagcagggccctgtgcctcagcctccctcgagtggtccagccctccctcagcatgggtgctgaagaaatggaagcctgtgctgcctgtcagggacctgccgctggagttttacccccaccccatccgcgttggccacccaggcccctgtggcacgggtgcatggaggcctagtgaggtcagaaaccgtccagccaagggtggggggattcagggtgggggaacaggcagccggctccgggagggcttctggtgtgagtttggggcgggccggcccactccctggagttggtgctgtcagggggtcctaggcccacctgcctcccacaggcctctgtactcactgggccctggctgttggttgggggaagaaatagagacagatccctggcttgcaaggactccaggtctggggtcagagcgtggacaagtccggagggctctgccacccggcactgctgaggacaggggcgcatctgacagcctcctgcctctaccgagctggcttcggggcagctgggcctcccggtgccaataccccccggctgttacctgcctgtcacctgggcgagtcacctggagccgcgtctgtgacatggaagggaacggttacaaaagccacatcatgtgagatgtgcgggcccagccaggcatctggggcagaccagcgctgcacacacacaggtgggatctgtgcccggtgcccagcctggcatacaggcaccatcccggagtgcccccatgggctgtgccctgacagctttggaggtaggaattggggggtgaggaccctggtgaggacaggcaaactcagccatcacccccctggccagggtactacacccaccccacaccagggctgggggctaggggtgggcacagcttgggcaggccatcctgagcgccaggcctggtccgcaggccggctggagggccctgccctgccaagcgggactccggctcctctccagcatctgccgcccggggtgggcgcccagggccaccagccctcgcggctcctcccctccccccgtgcgcgactgtcattgttaccattgcttggcttctgccaaagcagccgcagggccaccgggtgcttgaaccttctccatggggcgtcctggcggtggtttgagccgggcagtcctccccgccctccccagccccccagccccggcacccggccgttccactgcgcggcggcgtggggagggcgcggcgcgagctgagatggttccgcccggcgccgcccccgctgcccgccgcccgccgcccgccgcgccgagcgctcccgcaggatggcgcgggccaagctgccgcgctcgccgtccgagggcaaggcgggcccggggggcgccccagccggcgccgcagcccccgaggagcctcacgggctcagcccgctgctgccggcccgcggcgggggctccgtgggcagcgacgtgggccagaggtaggggcgcgcgggcctgggaccccgcttgtcaaatcgcgcttgcatcctgctcgggctgcgctggctgggctgcacctgcgttgcctgcagcccggggaaggggggaggccagggtctcccctcctaagcccaccacagcgccctgcttcctgccgtgtctccagcctttgagagcctgcgggaccccccactccccccacgtaggggctgaacacggcggggctgcaggtgagactgcaggggagggaggaagtggacccagctctgtgccaggcactgggggcccctgcccgagccctgtcccacaggagccccgatcttgttggtgccagcgccactttgggggagacagggccggcatccgtgtccccaccccagaggtgagtgaggccgaagcctgcacagcaggtcagggctgggacacagggctcaggctgcctggggagccctgctgtggggtatgcttgggctggtgtctggtgcttgtggcaggaggggtgcaggtggccgtcctctggggatggttgggcaggtggagcagcgcctacccctccttggctcttccgcaagctcagggtgggacacagcacccccatttcacggatgaggacaccaaggcctcatctgcagcagcaaattcctagccagcacctttcccgtgccaggagccgttctagatgctgcgggggaaatgaggatgaatcagacctggcctggtgtgggcacatgtcgtgggcacggaacctgttcctgggagagagaggtgcaggccaggccgggacagaagagccggggactcctggggtgctggtccttccgggacaagcatggggagatgggcaggggccttccaggtagcggaagcagtgagggctgaagtttcatcagggctggggggcagggctggcagagcagggcctggcagatggcagggccaagggtgcccaggagcctctcccttacccctcctacctggctgtgggctgttgggcccctcccggcacccccatttctagctcttcacagcagccctgggggggcactgcccagcttagtcccatttgcagaagaggcggttgaggctaaggtcaagcacctggctgcagcctcctgcagctaatgggcagcctccgccagggaggcgagcactggccaaggccctggggaccacagtcccctccccctcctcttcctggatgctggcgtggggggaagggggagggcagtgatcctggaagccctgctacagccagccctcaaggggcaggttccttgccccatccctgttcctgcagggcccatagggatagcaggttctgcccaggctgcgtctgagcccgcagtgcagtgcagcatgggagctgtagcctaggcccacctcctccaggaagccctcccgcatgttcatcccagccctgtgggctatgcactgtgctgacggtttcacatgggtggcctcgctcatcccccggcagcccctgaggtgcagagtccttatagcctccttttgcagagcagaggtaatggagctgctgcccagctgggctgagcagcccctgggcctggagcattcatacaacagggctcgcgctgagatgagtagggcaggctcctgagcagaggacttggtaggtttgttcttctggagtgtggatctcacggaaaacacaggcgaggccagagccgagaggcaggtgtggtctgccccagggagctgggggtcgcagtgcccgcagctgcctcatcctgcaggcctgagctgccttcatgggccagccagggtcccggctccctcagccctctcagactctgcccgtctcattgttgagaagacagtgaagcagaggagaggtctccactgcccaccagggactgagccacaggaggcgttgtcgggggagggcacttgctcctggtgcccctgcccagagctcccctaacaccctgtgcccacttatgggtgctgcaggctggcctctgtccctcctcccacacagtccagctcccctgaccctgagctccggggtctcctccttccttctcactcccctgtgtgaggctggggagggattgggagctgattagacagcgtggcttcagtggtggggtcaggagaccatcactgcaaagcttttgagtaaaagtgcccaatggggggacagggcctctcagtgagaccccgctgcatcaggaggccctgagcccctctgtgtccctaccacccctggacctaggcggccttgcccctcccagctggggtctcccagcctcatgcctgacttaaggcaccaacacagccccctccccacatcctctccggggccgttcccccagccccatcctactcttctggccctagcactgcactgggcagggggcccatgctgaggagctcacctggactgcgtgttctgcccccacaccatccctgagggaggtggggcacctctctttgaccccagggagcaagcctgaggcccccacacagcctgaggtctagcgtggtccagttctcctgccactgtgcccagcaccccagtgtctatggcagccacaggtctgtcagtccagctgtggcacgcagagccccggccccgccccaagcccctcgccctgatgttccttggctcaggagatggtcccctgagccccccgaccccggacacccttgccaggacctccctcccctgcagatccagaccttcatggttgcccctctcctctccaagaacctggctgctcctccatgcccccctcaccccgggcacacgcccggcgccccaccctcttcatggtgttcacaccggctcggcacaccccttcccctgggcctgaagccccccttggagcttgccccagatggcctctctcccaggagggcccctcctgagtatggtcccgtcacccctgaggtgcccccgccctcctctggctttgccccgctccgggctgcacaccttctttccacacagtgctctgcgctctttctcccccaccctagaatgcacctgtttgggcggctggtttatccttgacccaggagcgtgcacagggagtgcccagccgcaggggtggggctgtgttcccaggccctcgggggacaccaggccctgtctttcaggggtgtttgctgggcccacacctcatttattgtctcacagtcctggaggctggaagtctgagctcaagatgtgggcagggctggttcctcctgaggcccctccccttggctcgtagacaccatcttctccctgtgtcctctcctggccgtcccgctatgtgtgtctgtgtcctcatctcttcttataaggacaccagttttgtgggattagggcccaccctcatggcctcactttaccttaatgaccgctttaaaggcctcgtctccaaacatagccacgtgctgagatactgggggtcaggacttcagcatgttagcctgggggcacaaaactccgcccgagataagggggtgcctttgttgcgtgtgggaacctgccgggacctggctgtgctctgggcattccccaggtttagaacagaggctcccaaactcccccaagcagggaaccccctcttccccaaagcatcttatgggacttggggttgtggggtgcagagggacactccactcttcccccagtcagcccagaggggctcctagagagcccagagggtccctccctctcgaggctggcagctgagactgctccttatgggcacaagtcaagtggggatggcccctcctcacctgggctccttcccgaccccagccccaggctctgcaggacacagcagggaagcagggctaagcccagatccttccactgaccactgctgccctaagcagggctgagaccctctcctgggagggaggtgacggcttcgggtgcaggttctgttcgtgtgttgacagcgagcctggccatgggaggtgggtgggcccagggatgaggccctcaccacttggtcccaacagctaagttagggctggcagagatgctgagtggcacccaggccaccaaccgctgggacaggcgagcatcagggtcctcctggaccaccgtgggtttggatgctgcggatagaggccgagcttcctcagctcccctcccctcccctcccacccctcaccctgcagttcacttggcagggtttgtgcagccgacagaccttgtttgcacagagctccagtcctcagccaggttctgcacaggccagcaaagccccaggtccctttgcccacaggccgctgccctccccttgggagtccctgggtggatcctctacccatgtgcttggtggccccttccaaaatcagtggggacggctgtcctgggcctcaggctgtgggacatctgacaggtggtggcttcctcaacactcagacaacagggccttgggcagagtagatgggaggccccatgcgcagctcagtagacactcagctagggctatatcgggagatgcagagggacgcggccagggagcacctgggagagaggacacagtgggccccagcagggaggagcccagccagggcctggccacgccggacctgacccacaggccaggcagacccgctgccatgggctgaggcactgccaggctcctgccagcctctgacacggctgttgccctggctcagagcttcaggcagcaccagggtccagcgagagggaagctgagccggacaaacacgcagaatctgccagaagctcccagcaaacccaaagatcagccccattaagcctggggttctgcctgctccgtgtggagctgctggcccggccaccatgcccagccaggacctcggccctcccaggggtgtgtccagcttctgcctcccccagggaccagaagctgctagggctaggggcaccaggagggcacctgcccacccggagagcctggcgtaggccgatgagcgtgtgcctgtgaccccaggctggcttcctccttcctgcctgtgtggcctcagaagactgtggtcctctctgaacctttgttttcttttctgaaaacatggttgttttgaggattaaaagagagaggtccctctgaagtcctttgaggaggagctggtccaggacaggcacatagtcagtgaaggcaaagaccctcggcactcccaccctgcccagcccccgaccaggccggccctctggagccctcagctcccatcagcctctctctccgagttccgaatccagtgcaggttgcaaccaccttggtgtcagccgcctgctggccagtctgggacaactggggccaggttgcctggggccaggctgaaggggtagcaggtgtgtggctgctggcgtggtggagggtggtgtgtccaggtcccctgagaggcatcagctctgagccctggcttgtcctatcgcagggagccttggggcagggctggcgggtgcctcacccgctggtctgggtgtagggcaggggagtggagacaaggggacagcctcgcagacctcggcatgaggtggagctcaccgcgctcgcttctggatgctggggaccacacatggggcctgggctggcttcgtgctgagcagggccagggcgagtgcccaaagcatcgtcacctgcttgcgtggctgaagggtggccctctcctcgagtgtggttgtcacccccccatctggcccaggcacaggagggtgcatgcgcggggacctccaagcgggctgtgctgctttctcgggccctgagggggccaggcttggctcagtgcccccacatggctctgcacatggactgcctcccacaggcacctctcccgtgcccgggggcgccaaaggtccgggccccccagagcctccctgcgtgtctgtgccccgcctggtcccagagcccaggctctccatctgtctgtctactgccttctccatccgtccgtctatctgtctgctgccttctccatctgtccatctgtctcccgcattctggttgatgcccctctgtgtccccaggcttcctgtagaagatttcagcctggactcctccctgtctcagtaagtgctttgcaagggaggaactgtcttggtggcagacaagggctcttagaggtgtgagagcccttgtttctggggggtcccctcaacccagcatcccctgcagctgggccagggccaccggcacctcccagtacccgctggtgcctctgggtcccaggtgctggggctggagacaggacagtcagtgtgcccccttggagaggagcctgaacttgggaaatgggctccccagaggtgggtggtccgcagcctcaggagaggcggatggcagagcaggggcccttgtcatggtgcctcccagggacaggcaggcaggcagctgctggggcctgggctctggctcagcccaccttggctgcctgtgccggtcgagctcccaggagctgggatgcgctctcggaggctgtgccactccccactgagccgccggggctctgccggtggtgcttcctgacttcagctaagatgtaggtcaaatggaagaggctttgaggccagggcagccaggccctgcgcactcctgggaagccccccgctgccatccaccttcagcgcggtctccaggaggggccccaggcttactgttccatgcacggcaccagcagcagaaaccaccgagcactcagcactcacggctgttgagcatccaggggctgcccggcacccatggtgggacctattattatcccaaaagagtcctggaggctgggaggccagtggccaccaccggacctctatcttcccagcctggggccccatggtgcagagggtgacgggtgtgagatagaagccgtgcccaccacccagggttctcctggccccaccccaggctcccgaggagtcgggtctcagacctgtggaggaaatggatgcgtctgcagccgaggctatagtgtgtgccgccgtggcacggtctggacacagccgatgttcctggacctttggaggcccagctggactcacccttcagccctgagccagcctcacctgggcccctggccaagctatgggggggccccagaggcccaagggtgtcaaaacagggaccccagggggacggagacatgtgtgtgccctttctgggcccttgggcgtatcagatgctctgtccaggctcagaatagctggccccagggttggcagtgggcctgaagcctgccctttcttcctgcctgcctgcctggtgctgggggcagaggcctgcgtggagctcccaggcacctgcctaagagccagccggcacccagccactgccagagagaggcagctcggacctcggccccatgtgagtgcagggctatggtgggccccgaggcgtgtgcctgacagcctggagctggtgtggagcttcccggcatggccatctgtgtatgactacctgccaggaatccctggaaggccagtcccccagcctgcagctggcctgccggtcagcagggagaactgttgccggcctctgccgaccccatttgtcacagggcggccggcacttgcctcagagggtcccgtcttggtgtgggggtgggcaccatgagagcccctttgcagcacaagggcctcctcaggcagagcagtggcgtgagcccctgggcacaggcaggcagtgggcatgggtacacgcggggcccaggcctggctcaacaccagccttgcaggactggggcagagccgccacctggggacagactcccctgctgccgctacagggagccccaaggacccaggcctggcactgcctctgacctgctgagtgactggggcaagcgtgggcctcggggagcctcggggggctacagcgggcttcagccgtgtagggacggggctggccaggagctgcctgtcctcacggtgccagggacaagttgttcgccatgggccagcgccgctgacggctctcccagggccagggccagggccagggtttggcctgtggattcctcagggaccaaggcctcaccccagggggtacacaagggactgtctgaggctccctgctgtgtctgggaccccctggccaggcctgcccgcacatgcccatgagtctcaccggccctgcgtcttgctcctgggctgtaggccgtgggcagtggcggtccctggtgtgccccgtcctccccaccagccgaaatgcagactggcagaggtgagacgggcccggccccctgtggcgagtgtgtggctgcgccctgagcccgcctcccagggtgggggtcgggacccgtggccggggatccgggaatgcagcacccgctgccgggtgtggactctgggaatggcgatgttcccagacggatgccgccatgagcgctcacttgctgtttgccagataaacaaatgagtgaactgatgagtggatgagaatggacacaggaagtagattcttctgccccgtccttgcctattgtcctgggagcctgcgttcacaggcaggttcttcctagccctgtgggcggctccccacgggagttctcggtcatctcccacctctcagggcgttccaccgagcccacgcccagctcctgggagcaacttgccatgagcgggtcccctgctttcccaccggccgggccctgacaggaagcggagagtgtggccccacccggggtcccgcctctgcccgtggtccacacctgtggcttcatagcctgggagccacggtgagtcgaaggcctgcgctgtctcttttggctgacgtgggtcctgatggtttgagcagacgggtgctggcccccagatgagcaccggggcagcctctcaatggagcctgtgagcaagggcgtcgtcccaagagaagcctgggacacagaggccattgtttctatctcagcacagacagagctcctccacctgtctgctgtcctcgggacacgccgggagttggtccctgtcctggttcccattccccagggaggaggcccaggcgctggcaggtggaggggaacagccgggcagaggagcggcagggcccgtgtagcctcacagggctcactgggtctcagccctgggcctctggctgccgggccgggagcatgctgtgactctgaggagctagcggagccctccaggctaccagtgtccatgaacctctgccgcagcaaggaggcttgggccactctggcaggaaccaccaggcgggtctaccccagccgggtcggcctagagctgaccccagccaggcctctgcagccacaggctccctccccaggtgccgctgcgtgactcgtgaagacagatcgcaccagtcacctgaaatacactgcaggggaaaatggatcgttttcatctcctttttgtggtggtgctcggagaagtgggcggccacagttcattaaaaattaaaagcagaggaggcttcattaatgtctctggcatcgcaggctccagtttctcagggagtctgccaggccccagcgtttctctgcctccacgtggagggtgccatctgtgcaggtgggcactccccgctgagctccgtgggggctacccagattggggcccacagcccctcggtgaggggaccagggtctggctgggctctctccctaaggggggctcagtgctcttgtttctggggtgccaggaggaggtcaaggggcctcaaagagaagctgctgggggcgcgactagtcccagaccagcagccctcgaccccacggtgtctggggaggaacttggctgtccggaagccagtgaggtcctggcaatgggcagggttgcatctctctgtttcctggctgtggaggccaaggggagcccagggggacgcattggcttcgtggtcttgccaggcagctgcagaggcccaaggcccagaggtccagctgcaccaggtccacggggaggtaaagggaggagcaggagctccttggaggcctatgaccgatggagggttgggggcgtggccggagctggctggggagatggagctggctgggtgttgccttggagtggagtgtggggcgatggtcagggcggagctgggcacccccgcctaggaggagtggccaggcaggagcccaaggcccagagggaggtcagagggaagaaacatcctgagggcagagctgcaggacctgctgatggctctggtctaggggaggggagggtacaggagcctgaggttggtgggggatggttggcctggtgccatcaggaatggagaattctgcctcggggagggggagccggtggatgccagagggtagagtggggctggacggctgccagcagagccagttgggaagtcacccctgcgtggcacttaattcccgggctgggtgggagagaggcacctgtggcaagccctggggtcccccaaggagaacggagcggaggggagaacaacagagctcggggaggccgtgctgagaggcccagggagtagaggagattctggggtctggtcaccgtgaagggcatggtgaggctccctgccggagaggagagggtgggaggcagtaggagcaagtgaggtgagcagaccccccgcgttgaggcttttgccatggagggcgggacagtggtagggaggcggggagggtgggttcctaagctccatgcttttagggcccggtggtccctgggtaccgccggcagtgaggggaggagggccttgggagaccctgggctcagcccccgggggcagctcccgcctcagccctgcctgcccccctgacatggccagaccaggcccagcgcagcctggacctccagcatcctgtctgagcgaaagacatccattcagagagaaagacggcggacagaccccccaaagagacacttatgcagggaggggcctccccagccagggctccaggcacacagaggagaggcgtggggcaggaggaggggctgagggacacagcagagctggctacgctgaggggttgcagggcatgatggggcactctgggggcagactgaccccccattagcactggttcagacgccactggtctgtgggaggcttgtcccactgcctggccccaggagccctgagtatgcccggtggggtgacaagcctgcccccaggacttggcctgagcctcctcttcagcatggggcaagggcacccagagccacacgcctcttcctggaccctggacccttcttcatttcatttatttatttattattcatttttgagacagggcctcgctctgtcacctaggctagggtgcagtggcgtgttgacggctcacttcagcctcaaactcctaagttcaaacgattctcctgcctcagcctccccagaagctgggactacaggcacgtgccgccatgcctggctactttttattgagacgggggtcttgctatgttgtctaggctggtctcaaactcccggcctcaagcgatcctcccacctctgcctcccaagttcctgggattatgggcacgagccactgtgaccggcccctggccccttcttgagctctgtaaccagcagaggccactggggcagatcaaggcaggtgctgggactctaccaaggcccctcccgcccgccccacagcagccccaccacgaaggctgctgcacacacctggtccacaccacttaccctgagtgttgtgagctttgggaaacatcgaatgttttaatcactttcggagatgttggggtcaccagccccaggccaggcagtgacatcaaattctcagattcgattgataggagccagtgcctgctcagaggggaggtgaggctcacagcccacccactgggtgtcacagcctcctgccctggtggacagtgacaaggaaggggttgaggcaggaacaggggttaggctggtgcttacctcccccgacttctggacagctgccctgggacctctgcttgaacactgcctcctccacgcagccgtccaggatccttccaggcagcacaagtcttcctccaggctccgctgtctgtctgccctactgtgttgtggccctcgcgctgcactgggcagggggcccatgctgaggagctcactggactgcgtgttctgcccccacaccatccctgagggaggcggggcacctctctttgaccccagggagcaaggccaggcaccggaggcagcgcccgagggctaaggcagccgactgataggagcagacgtcatggagggcacagggccatggctgggagcagccgagggtcccatggaggacagcccagcaccccagggcacgggccgtttagggggacatgtgctcccccttcccagagacggccatgcctgctgcaggaaccctggctctgggtcttgctcctgtctgtcctgcaggctggcagcattggagttttcctctcaggggatcattaaaccctggcctagcctggcccgcatcccagcacagccgggcacaggagaccctgcgtccaaggctggggcagtgggggcagcagccgtccacccccaggctccatcctactcttggtggccagcctgggtcatgcaggtggccacggtaggtgagccacgcaatctccccaccccacctcaccccacctgccacccccggcaactggcacactgtggtctcttctcaggaccgccggggcccggttggaggccagagggctgggtggtaggccccatgagcagcggcgtgcatgggggttgaggggtgtctgggaaccagtggctggggccataggtgagcctgagggaaggggagcggcactggctggaggggccccacagctggcctgcgaggtgggtgcagggatgaccacttcccaccagcccatcttccctgacagcccttgaggctgcagtggcccatagccttggagcacagccaccagcggcctccagacagagcagctgaccatcagctgtggacagcctgtatgacgcaggtcctacggcatgttctgagcacttgcggtgtcctgagcaccatccccagggccatatgggcaactttacctcctggcaggtgccctgcacccacggaggcttgggcgagggtgtgtggagtgagtgccgcaccctgagccccaacagggcggcctggagggcccagactcacagcagcgagtgcccagggctgcggggaggaatcttcagaggagccgaggctgggtcggctgtctgctgggtgtgacggaccccaggaccctcctgggtctgaggtggagggagtggtcctgctgggccctgacttctcaactcctgcagttggaaagttggaagaagtcagtcaggttggggctcccggcgtgtccccagcctgagtccccactggccctgagcctccatgcccctctctgcttcttcagggtccaggtggagttctacgtcaacgagaacaccttcaaggagcggctcaagctgttcttcatcaaaaaccaaagatcgagtgagtggggtgcctggagggccactcccaggcagggaggtgttgagggcgccgggagcaagcctggcgatggcgaaggctggggctgtgagctcagggagagtccatggggtgggagccacctgagtgctgcgggggcatttggaagcagggtgggggtgggtctgcatggtgagtggggcacagggtagccccctcttagcttcacactgtctgagaccagggcctctgcagcgggactcgtggggacaccgtcctgcctgctggacaccaggctgggtggggcaggaccatccataggtgccagcaaggtctcccctgtggccacgccctgcctcccacccctccatacagccccaccctccccaacagctccctgccccgttcccagccagagcttggcatcatgctccccccgggattgcaggtgaggacttggggggtttccccaggggctccacattcacctgcctgctccccaatccctatcctgtcctccaagaagtggagtggcctctagaaatcccctgcaccccaagttcaggcacctgtaaggtggggccagggacagagcctcttggaggctgacccaagagcatcagcccccaaacaacgcagggcacacagagctctgcgtgcagcccgggtgtgggaggggagcccagctggggccatccagagagcccagccagacccgggtgcaggccctgctccccgaagcccagagctgggtcagagccctgaggccgctgccctccccgcaggcctgaggatccggctgttcaacttctccctgaagctgctcacctgcctgctctacattgtgcgcgtcctgctcgatgacccggccctgggcatcggatggtgggccacgtgcgcggccgggcgcggggtcccgggtcccagggctgagccttcccactgggccgttacagaagctgatggcgtccctgggggagcccctgtgcctggggccttggggagtccttccttccccagtgcctgtcccaggatggaaaaagccaaggactcagtgcccaggctcccgcagacggctcagcgtagggagaagggtccccatcgtagacttccagctgtgcccttagctactagcggacccagaaggtccacggaaaggctggagcatctaggatgtccctccctgcccccacaagtgtctctgtgctcacttgagcttctgcaggtcagggaagggggcgcccagtccccctggggaaagcccctacctgtttctgggggtgtcttctacccctcctgggtcccaaggagtcccctccccagggcccatcgtcctggggcccatgctccagggacttctgggagaggtgctgggggcagcaatctgcaccgtcaggtccttaccagcctcgcagcagccgcgatgagggtgggtgggaacttgctctctctgagtcctgttctgtctccttctgctgttgctgcttcgagaagtggcggctgctgggacctactctgtgcccagcgtgccttggcaactcctgctcacctctgtgtgggtcccaggggacatcagcagggccctgcggccctcggtgccggcctgtcctgagtcatgtctttggggaccagcccccaaccccaggcctctctctctggctccctggcactcggatgctgtgctctgtgtcctgtttcttctctcccctctcctgcctcgttcccttgttcactcgtccacttgttctgtcctgcctcctggtttccagaaatcctttctctgaatgtccttccgaagatggtgtctccagtcctttcctgttttacgctttccccatctgctgggcctgtttctcccgagctcacattgggccctgactttttcatacccgtagatttcctcaaatgtctggtgccgtttatgcgtaagagtgctcaggcgctgaggggctctgcagccacgtgtgtggtgtggttgtcaccatccagccatcggggtgaaccctgccagcatgctggtcccccctctggctgcgcagagcaggttcttccctggagagaaggcctgactgccgggagcctggcttctggggacctaaaggcgtccatgtaaacttttgctcaatcccccttttcacctgttaccccgtcacaagggggcctggcatccccaggccagagactttccctctcctaagaatgaacctcctgtctttgtctaggtcagaagagggcagaacccactaggagagtttgagaaggagaactgggccctgactgactctcaagctactttcctaggtcactccccaccccaagtcccagggtcccctggggcttctggtttcacatccccacagggcccaagtctgttgtgttcactgcccgtcccctagcacagcgtccaggacatggatgggggtgggaagatgggtggatgatggatggatggttggatggatggtggatgatggatggatggacggacggacggatggacggatggatggagtggatggagggatgagtggatgggtggataatggatggataggtggatgcatggtgggtagatggatggatggatggaatagatggatggagggatgagtggatgggtggatgatggatggatggatggatggatgatgcgtggatgggtggagggatggatgttggatggagggatgagtgaatgggtggagggatgagtggatggatggagagatgagcagaaggatggagggatgagtggatggatgatggatggatggatggacagataagtagatggatagatggatgagtggatggatgatgagcagatggttggagggatgagtggatggatggatggagggatggatggatggacagatgagtagatagatggatgagtggatggatagagggatggatggatgagtggatggatggaggaatgagtggatggatggagagatggatgatggagaagtggatggatggatgagtgagtggaaagatggatgggtggatggatgatagatggatgtccagatacttctgtgggcacttagtttgcaacaaccccattaaaaatcaggggaaagtggatcagttgaaagaaaagacaagaggagctggccatcaccaggggccttgtgagcagtgaggtctccttggtcagtgaccctggcatcgatgatggtctgggacaaaactagcttgttggccatgccctggtcactgtcgcagcagctggttccaggtggcttgggggccgcttgtcctcagaagcagcttagggacgtgcctcttggagtcagcacctggggataggagcttcctctccctgtctcttgaacactgtccagccttgttcaaggcagctctggccttggggaggccctgagtgttcccacatgctctctctggccatgtaaggcccctccctgggagcctgttgtcagcacaggcagaaccagtgtgttgctgttgggttggggctacccccaatcccgcagatgtgggatgtaccctcgctgtctgcctgctgggctgagcagtggccagcccagcattcctcagttagaggccctcctgcaggatctccgcagtaggtggggagggggcaggctgccttgtctcccaggtgttagagctctgatgtgggggttagccccgggtgggtgagtaggaggcccccatgaacccgagcctgtggaagccctcgggcagcaagtccttgcagtggtgctagaggggccagggccgggccagcgctgtgtctctccacagctggggctgcccaaagcagaactactccttcaatgactcgtcctccgagatcaactggtgagtccacactccagctcccaatagccaggcgctcagaggcctgggaccagggtggggtgggatgagtctccactccagctcccaatagccaggcgctcagaggcctgggaccagggtggggtgggatgagtctccactccagctcccagtagccaggcgctcagaggcctgggaccagggtggggtgggatggacagaggctcacactgcctggtccctgagggataatgcccagcctcaacccagcagccacccaggttccccaggacacatgtgcccttagccctggctgtggtcaatgctgggaatgtccccttcaagaggagcagtttgagaattgtgagttcccagccagttccacagcctccacacgctcttcctgagcccagtgagagatgcaaggaagcccgtctcccagtgaggtggcggcttctcttccagttcaccatcttacggcaaaggaaggcacctgccccgggcaggcagtgggaggtgactgcggggcaactggggtgcctggtccagagattaagacctgtccccagaacctgcaaggcatgtccccagtgcctgagcccctgtccgcatcaggcaaggagcagatggccccctgcagccatgcccctgcctgtttatggatgaggaactgagactcggggaaacccagggggctgtgggttagatagcccgacctccctgctcttagggacattcatgggagcccggcttcagggacccaggtgaaaaaaacaggctacccagtctggggcccaggctgctgttgcctgtcgtgtccagagaggagaagctttgccacccttggtagtgtcatgggctgcaggccaaggctccatggagactcagccatgggccctggcctcacaagcacctcctggggggcctgcctgctgcagtcctgctccatcctcctctcttggggaaccttcccttccctcctccttatggtcccctccaaaggcaaggtaggctcctttggtccagccccaatgagcagcacatgcttggggccagtggaggccaatggttatggccccagccccagggtggctgggggacactagtggctgtggtgccctactgtgctgcctcctttctcttcccagggctcctattctgtgggtggagagaaagatgacactgtgggcgatccaggtgagtgccctaccctgcccccctcccgactgcagtggtgctcagtaagcactgaggaccaacccagactcagtaagtagggaacccaggctcggtgagcactgaggacatacccagtctcagtaaatgctaagaacaaacccaggctcagtgagcactgaggacagacccaggctcagtaagcaggggacccaggctcagcaaatgctgaggacaaacccaggctcagtaaacaggtgactccaactcagtgagcactgaggacaaacccagcatttactgacccaggctcagtaaatgctgaggacaaacccaggatcagtaaacagggacccaggctcagtgagcactgagtacagacccaggcttagtaagtaggggacccaggctcggtgagcactggggacaaactcaggctcagtaggcaggggacccaggctcagtgagcactgaggacagacccaggctcagtaagcagggaaaccaggctcagtaaatgctgaggacaaacccaggctcagtaaatagggaaaccaggcccagtgaatgctgaggacaaaccaagactcagtaaacaggtgactcagggtcagtgagcacttaggacagacccagcatttactgactcaggctcagtaaatgctgaggacaaatccaggctcagtaaatgctgaggacaaacccaggctcagtgagcactgaggacagtcccaggctcagtaagcatgggacccaggctcagtgaatgccaaggacaaagccaggctcagtaaaacaggtgactcaggctcagtgggcactgaggacaaacccagcatttactgacccagggtcagtaaatgctgaggacaaacccaggatcagtaaacaggggacccaggctcagtgagcactgaggacagacccaggctcagtaagcagggaaaccagactttagtgaatgctgaagacagacaggctcagtaagtagggaacccaggctcagtgaatgctgaagacaaactcaggctcagtaagcaggggtcccaggttcagtgagtgctgaggaccgacccaggctctactgctctccacacaattttcccagggatccctgaggacttcctgaccccctctgtctgcttcaccacctgaatctctccctccactgacccatctgtgtccttgctgttttcctggggtggagagtgcacaggccaggtccctggctggctcctgggtgggtgacctagttcatgactgggtcccatgtctccatctgagaccagagagggtctttcacacccacccctatcatgttccagaggggtcagtagatgcctgtgctgagaggggttccgtgggggaaaaccaggtccactgagcctccatctccgttccctccccaccagggcagcgggatagccgctctctggtgtcttcctgagcgtcctgcctcacccactgcaccctccatcccaccctgggcctagagtgggcggcccaggaggaccatggtttcaagactccatctgccctcggccctggcgggggtcagccaaaggccttggtggctaagactgtcctgacatgggagtgagggtcaaggcagaccccagacacacacctggctttgtgtggtacctgcccgcgaggcctgtggggtcaggccccagccccagccccggcctgctccagagctccttccctttcctgactcccaggtcatcgtggccataataagcttcctggagacgatgcttctcatctacctcagctacaaagtgagtgcctgcccgggatggcacctcacagggggtccccaccctccccaccctccccagcctcccccacctcccccaccctctcctatttccccacacttcccagcctcatccactgacctccagggtgggctcctcgccctcttccccaccctgcccctcccctgctgggccccacccccaccctccatcgcccccgctgataccccccgtttggccccagggcaacatctgggagcagatcttccgcgtgtccttcgtcctggagatgatcaacactctgcccttcatcatcacggtgggtgagccccagctgccaggagtgcgggccctggagccccagccctgacctgtccccttcacagatcttctggccgccgctgcggaacctgttcatccccgtctttctgaactgctggctggccaagcacgcgctggaaaacatgattgtaagccggggcggggggtgcagctgggacttgggggggccaccctcagcctcaccggccctggaagacactgtgcgacgtagcctgccacgccccggccctggcatgacctgtagaggaccgggaagccagggcagcagaaactctgtctctgctcctggaaaacatccaagccaagccccgaatgccctccttccaggaagaaactcaatcccagagcttttcctaagcctggaaatgaaatggctggcaggaggaggagggacaaagtgaagggtgtgacaaacccaggccacgaggaccagggccacaaacaccaggccacacgcaaactggggccacggggccacaggcaggccctgtgctgagcatctcaggaaacctgggccacggggccacaggcaggccctgctctgagtgtctcaacatggtccctgtcatgtgaatggcagcagtcagttccctaggcgcccaccctctccaagccagaccccttgactcacagcccccattcctgccccagtggtaaatcagcgttaccatgctgggtccaagccttgcacactaaccagccctctgacctgagacttaagcctcatccccggggatgtaggcaagatggggacccgtcctgagagaggacagcagagggggtgccctacccaggtgcaggctgcccgtgggcctcagcggagacgcaggtgtggccaggcgtgggctgccccttgggcctcagccgagacacaggtgtggccgtgggctgccccgtgggcctcagccgagacacaggtgtggccaggtgcaggctgccccgtgggcctcagccgagacgcaggtgtggccaggtgcaggctgccccatgggcctcagccgagacacaggtgtggccaggtacaggctgccccgtgggcctcagccgagacgcaggtgtggccgggccgtctgctttccactgtccttctagtctctattcattggctcctggcggggtccacagtccctgcccgctgacagccaccactccttccacagaatgacttccaccgtgccatcctgcggacacagtcagccatgttcaaccaggtcctcatcctcttctgcaccctgctgtgcctcgttttcacggggtgagtgccggccgtcagtgtgagcaccccaggacgttgggagggcccgagaggcaagcagggccgggcgaggggatacagatgcctatgtccaagctatcggggcagaaaaggccacagtgcctgggctgcgggtgtcgggccaccaagctgggactgaggtcaggaggcagctccaagcccacgtccccagtacacgagcagccctgcagcccgactcctccaaggacagagatacccagatctggcttcctggtctatgccatggacgtagagaaggggactggcccctaggccaggtggggtctcttggctgaggcccagctgaaagcagggtctggaggcagccagggtaaaggtgggggtgcccagagctgcgagggcctccagcccacccaggcatgcccactgtgcccacctgcctgtgtcctcgtggagggctccatgttgctgctctgccttgggtcccagcgaggcctggtcaccacttcccgtccccaggcagggatgtcaggcaagcactgtgccctgggggagggagagtgccctgcgtttcccgcctcccttcccccctgcccctcatgacagactgacagacacagagctgagtgggcagattggggcatccatgaggatagcatctgggacctggcggcgaccccagccctgcccattagacctcccagcctcaggcctgggcgcttgtctggctgtgccgggcagaggcctgagtgtggtgggtaaaggggcaaggctctgagatgggggtagagggccagaccccaggcccacccctgtgtcacccaagcccacgctgatgacacagccctgcatcccctgctcccagagaatgttccagggacctaggagagagccacccggcaggcagggaggctccggggaattcgccgtgaacagaggccgccatgctgtggccaagctgcattgtcagccagcgtcaggcaggaggtggctccggcagagcttggggacagatgggcagggctgagggcctgatgccacccagctgtcaggagggcggggctcgcctggtgatgcacagctcagtctcctgggcagtgagggtcccgtgggcaggcaggatctctgaggggccacggccccccagctcctgggccccaggccgcccctcactgccaggggttgcaggacctgcggcatccagcacctggagcgggcgggcgagaacctgtccctcctgacctccttctacttctgcatcgtcaccttctccaccgtgggctacggtgacgtcacgcccaagatctggccatcgcagctgctggtggtcatcatgatctgcgtggccctcgtggtgctcccactgcaggtgggtcctctgggcaccagccctgggtggcaccagcaaagggacaggcgggtgccagtagagggagggtgccactgaggctgtggcacagtgcgggggccactcccaggaggggacagtgaggccaggcgggtggtgcctgctccgttgcacgcccccactgagggtctacggcgggtccggtggtgctcagcatggtgggtaatgatggagtcccgtgagctggcctcttccttctggggagatggtgggtctccagtgccagggtgacctgcccctcccaggcccaggcagagtgcagggaagggtcaaggtggacagccggcccatttcccatccacagccaggtgcagcagcagctgccaggcccacagggggcacacccccccggccaccccagtggcttccccgtcaccactgctgtggcccactgcccactgagcagaggaggggacggggcaagacctcagtgggaaaggtggaggcctggagagggcagctgcctcagggtgtgaagtgcttgggcctggactgcctccgacacctcctccaggcaccccagcccaccctggagggaccctgctattggggagatgggagaaggaggggacccctgtgggtggtggaacattttccaggaggctgggtaggaggaagagcctgaggaggtggccagggccttctgggagacagacccccaggtggctgcaggatgccggggagacagggcagtgctcctagggagcctctgctgaccccaggctcagccccagctccctcccgctaaacagcagtgggcgtggcccaggtatgggcccaggccaggcctggctcttctcctcactatatccaagccaaagctgtggcaccagctgtacggcccccagcgtgggccatgttctccacatctgtggcttctgttctcctgagttcagatggggccgtgcgcgtctttccatctggttgtcggccactccaccgtccagtgccacgagtcccgctcctgtgagctgcccgctcctctttggtcttccccctggttctcgctgatgagcgagtctctgtgttctagaagaagccagttgtgtgtggctttcccgttacttcctgctctgtgaccgcccctctcactttgcttacagaatcttccattcacggatggtcttgattttttttttttaaattagagatagggtctcactctcattttgttgcccaggctggcctcgaactcctgggctcaagcgatcctcccgcctcagccccagaaatagctgggattacaggcggcttggctttgattcaccaatctttcccttatggtttctgttttcctgtctttactgaaaagatcattccctccctcgatcataaacctggcttcctattttcttctaaaagtgtaaagctcgcctctcacctggagggtttcacccaccaggaaccaccatgtggggtggggtggagctgggttctcccctctgacgcctgctcccccacaccctgcattctgctcctttctctgcgcatttcataaccaggcaagggcgggagtgagggtgggagtgaggccaggagcacagtgtggggggcactccgtgcagtgacactgcctccctcctcggccctcctgccccaatccgtaaatctcacctcaaattctttaccttaattacttctgcaaagacccttttgcaaatgaggactcatcctgaggtgcagggggacctgctttcagggccatccttgaccttgctacgggcccccagcctccgtccctggtccgccccggcccacacttcaccacgtggccggcacctgctgaggccgctgcacccagatgctctgcagaggcgttgacccatccagtctctgaattccccacaagcccttccagagaaacatccgagaggcccctggcccagccgaggtcagaggaagggtcaacagggccaatgcgtccccctttcccctgtgggcccacggcccagccacacgcagctccaccttcgggctgcgtccggctcctctccccaccccacacaccccagagcgaagaggagccccagccccagccacccccagggtcgctttcaaataaagcaggagcgaagcgctctctcccgtgcttctcacgagaccgtggcacccacgggtaagggccaaatcgggatgtgcagcaggcctgttccatgtccctctgctgcgtccacagcctccgggccggggctgccttcccattctgctcctgaagcaccaggggagcccccacctccccttatctccattaagaagtaagacaaggccaggcgcagggctcatgcctgtaaccccagtactttgaaaggccaaggcgggaggatcgcttgagcccaggaatttgagatcagcttgagcaacgtagagagactcccatctctaccaaaaagtatgaaaattagctgggtgtggcggcatgcacctgtagtcccagctacttgggaggctgaggcaggagaatcgcctgagccctgaggttgaggctgtggtgagctgtgatctcgccatcgcactctagcctatgcaacagagagtgagaccctgtttcaaaaagaaaaagaaaaaagggccaggcacaatgggtcctgcctgtaatcccagcactttggagggccgaggtgggtagatcacttgagatccggcgttcaagaccagcctgggaaacatggcaaaaccccgtctctaccaaaaaataggaaaaattagccaggcatgatggcacacagctgtagtcccagctcctcgggaggctgaggtgggaggatcacttaagcccaggaggcagaggttacagtgagccaagatcacaccactgcactccagcctgggtgacagagcgagatgccatctcaacaccatcttaaaacaaacaagacaaggtgactccaggtgcaccaggtcctgcaggcatcacacctgcactgctcttcagggaaattcacggaacaaatgcgccatcgtcacgagaactgattgcatcgatgggtatggccatggtgacaagtgaccgacagcccagcagcctgcacgcagaggtgtcactgtagctcgagctccgtgtccacagtggggtccctcagatcagccactgcctgactgccttgtctcctttctcccgtgtggggcaggcagagcaggccctgagtgggacaagctctgagcgaggcagggaggaaggcgggggcagggcctgggggctggcgcaccctccctcccatcagccctgccggacctggtgctcaaacctgacccaggtgggcctgtgctgtccctggcagggctgggggtgattgggacagtgatgccgctgaccccaccgtctaagccgctccctgcctcctccgaatgactggaaagactgagcctctcactggcgatggtaagggtgggctgcagtgcccttgcccttggtctccactgggctgaatgcaccagtaaaagcccttgaagcagagtgatgaccccgaggccctcgctcacgagcgtccttctggaacacagcagcgccggctgggtctcctggaagtatcctccaggctcttcgatcagcaagacagacaggcagcttaaacagcagatatgtatcctccgccagccctggaggacagaatcggaagtggaggggttggcagggctgaccctatcaggccgctttgggggagattgtcccatgcctctctccagcttctggtggggccgcaatccctggggctgcttctacggtggctgtagaaacctcagcctctgccccgtgtgcacttggcctcttctctgtacctctcactggatttagggcccactccaacccagggtgaccttatcttaaatcattacatctgcagagacctatttccaatcagatcacatggcgaggttctgggcagataggacttttagggtcactacggagctgccgtcattagctccttcatcctcacggcggccctggagggttgccgtgccatgcagccatcttgtaggtgaggaagctgaggctcagagtgggggacgtgagccctggggtgtctgacagcagagcccccgccagcaccatccgcggggaagcctctgttccggtcgccctggagtcttgagcacctcccagctgtggaaactgttttgtgtggcaaggcagtggagcctcgtcaccggaacaaagcaggcgctggataagtgcaatgcgatggtcccatttagaccagacgatagcacatcagccatgcagcccacagtccatgggagctcctgccattgttcatttaacacgtgttcaacaagccgggcaccatgactcagcctgtaatcccagcacttcaggacgacaaggcaggaggatcgcttgagcccaggagttcgagaccagcctgggcaatgtagtgagaccccatctctacaaaaaataaaaataaaactagctggtcatggtggtgatgcacctagaatcccagctactcaggaggttgaggcaggaggatggcttgagcccgggaagtcaaggctgcagtgagctgtgattgcaccacggcactccagcctgggtgacagagtgagcctccgtctcaaaaaaaagaaaagccaaagactttcacctagaggccagtggggccggtgctgggaggccccggtggcctctgttttggatctgtcccctttgagtctcaacacccagagcttgcatctggggagcatgagccagggcaggcagcatcctcagaccagcagcctcaaaccacctgccccccagacataggctcctgacagtgaggcctgcgagtgcctggaagaccggggtctgaggcctgatttggccacgcttaccatcaagagagtgatccccaatcccacgggctgagcctccccaccctcggtttactccacagtctccagccgcccctcctcccttggtccccacacatcctgccagccgggcctgcgtcctgctctgtgtcatctgaggctatcagcatctacagtttccatgtgggaaaggcccccctaatgtaggtgtcaccccccggccggccctgccccaggcccctaacagacagtcgtgtgtcagggcccaggttcttcaccgggagccctcgctccccaggcctggtcgctggtgctcacctgtttctcacctgcagttcgaggagctcgtctacctctggatggagcggcagaagtcagggggcaactacagccgccaccgtgcgcagacggagaagcacgtggtcctgtgtgtcagctccctcaagatcgaccttctcatggacttcctgaacgagttctacgcccacccccggctccaggtgaggccccttaccgtggcccagcagacgactccctcccggcccctagagacgccatcctctccccaggactgcttggcaagtccctgagtcctctcagccttggtttaccccttcagtgagggcagatgcctccctcccggcgcccagagacgccatcctctccccaggactgccatcctctccccaggactgcttggcaagtccctgagtcctctcagcctcagtttaccccttcagtgagggtggcagcccacagtcaggtggaggggccctgcgggggcccctcttttccctcccaccagatgcaagatcacagtgagctctagggcccagaggtggtgggcacaaacacagtcctgaaggcagggccgcccgggcggggcaggggctggctcagagggtctgaccctccgcctggccggcaggactattacgtggtcatcctgtgccccacggagatggatgtccaggtgcgcagagtcctgcagatccctctgtggtcccagcgggtcatctacctccagggctctgcactcaaagaccaggacctcatgcgagccaagtgagtgctggtgggcggagggggtggcatgggggcaccttcctgagtcaggtcggctgctcagggctgaggcattgaccgtcgctctcctggccaatgagagccatgagagcattatagactatccccagggtggaccatctaggtggactgtccagggtggatcgtccggggtggaccatttagggtggatcgtctggggtggaccattcagggtggaccttctgggatggaccatttaggtggaccatacagggtggatcatctggagtgaactgtcaagggtggaccatccagggtggaccatctggggtggaccatctaggatggactgtctggggtggaccattcagggtagaccttctgggatggaccatttagtgtggaccatccagggtggattgtctagggtgggtcatctggggtgaactgtccaggggggaccatccagggtgggtcatctgggatggactgttcagggtggaccatccagtgtagatcatgtggggtggaccatctagggtagactgtccagggtggaccatctggggtgggctgtctggggtgggccattcagagtgaaccatctgggatggatctctagggtggaccatccagggtggatcatctggggtggaccattcaaggtggaccatctagggtggaccatctggggttggtcctctggggtagatcatcaggggcggaccgtctggggtataccctctggggttgtctggggtgggccatccatggtggaccctcttgggtggaccatctggggtggatcgtctggggtggaccatccatggtggaccctcttgggtggaccgtctggggtggatcatccagggtggactgtctggggtggaccctctggggtggattgtctggggtagactgtccggggcctcttgcagtttctctagctcaggtcccaggcccagatgagcaggaccctgcagccaggcccttgtccctgcaccatacccactgtggagcagcccaggcaagcccaacccagctaagtctctgtctcccttttcagggtgaggggcagccttttgcgagctccaccttcccctgctccaagtctagggaaatctttgtcaagttctagggaggctcaaaatagaacaggggcgggagagtcagctgctgtgagtcaaggtgggagaacgggctgaaggtagagttccttttaaagacaaattgacagagtgacaaggtaacaagagtatattattacctggaactttccagaatgtgtcggtgcactttctgggacactccctggaaaaccctcctggtgacattttcattccaggagcctgaatgtgtccttagtggcatagtgttggtggtactcccagggtctctcctcatcactccccctccccctgccatgtccagagccccaagctggaacccattgcctatggaacgccacctcttttgtggtcccgtccccagcacacacaggcgatgcacctgcttgggcccagggagtggggagagggcaagggcgtaggccattcccagccaggcagggatgagcccagggctgtgggtacagggcctggggcccctgcggagcagacagtggtgccagtgatgtggtggggttccctgccctggggcagaggcgacagtttcttagacagcgtctcagcagtgctaggtggtttctgcagctgggctttcaggagacccaggaggcgctggaagtgaccacccaggttcccagaccacagagtagccaggccagggagcggacagggtctctgcccccagattcccccctccctcctgcaccccaacccagccctgggtgaatgtctcatggcaacatacaagatgggtaccccggaagccagtgcccaggtcactggcacacacctaggtggggtctccaccgggcctgctcctgccaaccctggacccctagcccctgacgcctgggagtatggacaaatccaggcagccccagccagcagagccccctcgcacgacctcctgatgcccctcctacctggccaacttagggacaagctggggggttgcttcagagaacagggcctgggggaggaagggcaggcactgccctaggaagcaccatgggctcccactcagggccgtggccaggagaagctgtcagctggtgtctccaggacacaccatccatagcatctgaccccctgcccccacccgctgtcagcctgcacacactcgggggacaggtgtggctcctgacccctgcccccacccgctgtcagcctgcacacgctcaggggacaggtgtggctcctgaaccctgcccccaccctctgtcagcctgcacacgctcgggggacaggtgtggctcctgtgccttctcgagtgtcggagcttggagtctcctagggtgtccaggagtcctgacccggctcagagcctgccagggatgggccagggagtctggagaggcccaggatgcctgcgggggggggggggggggcactgggataccggtggggggggcacagggatgcctgctggtggagggcacacaaggatgcccgtgggggacactgtgatgtgggttggggggggggggggcagtatggggatgcccactgagggggcactgtgactcctgaccagcagagagtaggggcctcctcccgccttccatcctccccgccttccatcctctccgccttccatccagccgtcctctcagtctctttctgtgcacctgctgcaccagcctcctcccagaggaggtcctccccacctcacctccgcacccccggctgcactgcccacctcccctgctccacccacgctcaggccctggtgcattgcaggatggacaatggggaggcctgcttcatcctcagcagcaggaacgaggtggaccgcacggctgcagtgagtgaggctgaggccctgcccaggcgggaggggcaccgtggggccggggagcgggggtccctgagggaagagacctgccccaggctgccggtgccgcccagcagcccacagaggccagcccgtctgcactgaccaaccacccaccccgccaggaccaccagaccatcctgcgcgcctgggccgtgaaggacttcgcccccaactgccccctctacgtccagatcctcaaacctgaaaacaagtttcacgtcaagtttgctggtgcgtctggggcacacgtgggtgatggtgtatctggggcagggcacgtgtgcacacgtgggtgatggtgcatctggggcagggcgcatgtgcacatgtgtgacggtgcgtctggggcaggacgcgtgtgcacacgtgggtgacggtgcgtctggggcaggatgcgtgtgcacacgtgggtgtcggtgcgtctggggcagggcgcatgtgcacacgtgggtgacggtgcgtctggggcagggcgcgtgtgcacacgtgggtgacagtgcatctggggcagggcacgtgtgcacgtgtgtgtcggtgtgtctggggcagggcgcgtgtgcatgctgttgggtgatggtgcgtctggggcagggcgcgtgtgcacacgtgggtgacggtgtgtctggggcagggcgcatgtgcacgcagttaggcgagctgtgtggggcagcgggaggggctggccctggagctcctcacacaagcacaccaggaggtgctggaggggacggcagacccccatcctcaccgcatccgagaagggacctagggggtccaaactcttcagatgaagtcttatgctgggatcctggggtcagtgaaggcagggtcagaggtcaggtgggggcaggagcaccgtctgatgagcacctctatgggcagggaccatgccgggtgcccggggaacggggggcaggccccatgccaggtgcccaggggacaggggtcagggcccacatggagtgcccaaggcacaggggccagggcctgccccgccctggaactcctcgctgagctgggagagaagcacaggagcgatggaaggtccaccgaggctcagaccaagtagggggttgaggtccacagactctcggggcagagatgctgaagccggacagcagacacgggggtgccaggcaagggtgcatctgcatagcaccttcaggaagtgagcaggtactgtgggggaggagagagccggcagagcggcaggtggaccggcctcccccactgcccgcagaccacgtggtgtgtgaggaggagtgcaagtacgccatgctggcgctgaactgcatctgcccggcgacctccaccctcatcaccctgctggtgcacacgtcccgcggccagtgagtgccccgtgccccgggggaccgacctccatggcggggccggcgcagggagacaacgcagggcctgcttgggggcggggatgggcttcccagaggaggggcacatggcgggcaaaagtcctgcatagggaggggatccatgccaggggaagcagaggggggcacctgcagacccagccggggagaaggggcagccatggccgagggtgacgctcccctggccccgccctggcccacagggagggacaggagtctccggagcagtggcagcgcatgtatgggcgctgctccggcaacgaggtgtaccacatccgcatgggtgacagcaagttcttccgcgagtacgagggcaagagcttcacctacgcggccttccacgcccacaagaagtaaggccgggctgcatccacagggctggcgctccagggctgctctgctctgtgccctccccaccctcccggtcaggcacaggggtggccctggggcggggctgcagagggctcgggggagggcatcaggtcatcctgcctggcgagggcagccgcaggactgggctccgggtccacataaaaacctgccacgcggctcctccctgaggcttgtgggctgaccccagctctctggtccccaacacctctgggacgggagggctcagccaaggtccctgaccccaaatggcccccaggaggaagacgcggagctccggtggggactctggtgatttgcaggaagggcaggcagggagcgggacagggcgggtgagcggcggtacctgaagttgccggtgcctctgcccaggtatggcgtgtgcctcatcgggctgaagcgggaggacaacaagagcatcctgctgaacccggggccccggcacatcctggccgcctctgacacctgcttctacatcaacatcaccaaggaggagaactcggccttcatcttcaagcaggaggagaagcggaagaagagggccttctcggggcaggggctgcacgagggtccggcccgcctgcccgtgcacagcatcatcgcctccatgggtgagccgggacaggcgcgcgggactccctgggcctgctcctttggcgggagaccaggcgggacaccggcaggtgaccaggtgggatgggagaccaggcaggacagggggaggtgaccaggtgggacaggagaccaggcaggacaggggcaggtgaccaggtgggacgggagaccaggcagggcgggagaccaggtgggacaggagaccagaccgggcagggcaggagaccaggccaatgtggcccccagacccagttcctcttggcctatgcctccaaccttggacaggtggagtctctctgggcctgtttttgaatctgtcaaacaggtgtccccgccttcctgtggccacctttgtgggcattgctcactgtgtccaagtgcctgtccaagtggggccgcccacaggaccggttggcagctcaaccggaggctcctggtggtcccatcagcccggagggtctgcttcacgtgtgtccctcaaacagtcgggagtcctgacgtccactggggccaggagttggcggaatgaggtcgcagtggccgagggctttggcctcttctcgtgccttcagggatcctcccggggagccgcttaccagacagggtcaggctgcctctgagcaggtggaggcccatggcccctagggcaccacccatagtggtgttgccccctccccagccaggccttcctgggggacagttagggagcagggccaggccaggaagccactcaggccacagacccacagccgggccagcatgttgccacctccgtacagtggcccaggcagaggctgaccctatggggcaccccagcacgcccaccctggggtgttgttacacggtggctgctggggcaccaggtggttcagtgcagtggggcacagtctccaagacccagagggccctggggtttggagacgtgccgatgcggagcccaccacctgccagaccccgcaggttcccggccgccgtctacggcagctcactcggggggccgggcctggccgcagggcactggggaggcaggctgctgctgcctagccacacctccaccttcacctgcgcagtaggcactctgcccccaggtggcttccaggaaaccagggtgactcgggcaaccctcactgtacccacagaggccctgggccatacctagaggtccacaacccccatctggcagcctggggtggtgcaggagtggcagagtctgctcccacaggcactgagtcacccagctgctccccacctggccccatccaccaggcagcctgaccagctgcacaggcccctaaggctgagacccccgagcccagaatcagccaaccccctcctcaggcatctggtgctgaggccacagcagctggcctgggtggcaccgatggggcactggggccccctgggtcttcgctcaacatcatcgccaccccagaggccactgtccctgttgtatggagggggaaactgaggcatagattgaagctcctcagctggagcacaggagccaggccatgacagggtctccagagctgactgtgttcacctgagctctgggaactcgccgcccatggagggtgggagtcgggctgtggccaagcacagggctctcttccagggacagtggccatggacctgcagggcacagagcaccggcctacgcagagcggcggtgggggcgggggcagcaagctggcactgcccacggagaacggctcgggcagccggcggcccagcatcgcgcccgtcctggaactggccgacagctcagccctgctgccctgcgacctgctgagcgaccagtcggaggatgaggtgacgccgtcggacgacgaggggctctccgtggtagagtgagtgctgccttggagacggctcccagtggggggaggagccgcccatgagtgcgggggatgggtgtcggagcatccttggtggtcccccatgctctgaattgcagcttctggacagctccgtggaagtccttgtttgacaaatgaaatcttcagggggcccaacacagacatcagggccacatcaccctcgtcgccggggagcacttttgagtgtcactgagatggggtgtgctgggctacaccctttccagttggggctgggggtgcagagctaattggagcgaggcagctcactggcacaggggctgtcaggcaccaggcagcgccaccctcagagaggggcccgttcccacccacctcaccagccccatagtggcccggccactctctgaatcaggatggagctgggcagtgaccccaggccatcctccacccagtgccctaggtctcccccttagcactacagagcacagaggggcacagcccgcctgaccaggggtgggtgtcctctgagcagggtccctgtgcaacccctgggcaaggcagtgtcccagcctggaaaccacagcccgtcctgagttgttgtcctggcctaggtttggggccaagtcctcctgcttcaaatcatgagacaggaggccccactctgttctcagcagcttctgccttttggcctcagccaggacagacaagtgccccagctgcagggcccgaggtccatcctcagcggggctgcctcactctgtcctggcctttgcctctggggtggggtcaacacactgtaatgacagcccagctgctgggaaacagccctgaaccattgcatgtgtgtgttgggtgctgctggggacggggtggtggcctgctggggacaacagcctggctggacaagtatgtgggtaagagcccaaggccagagtgcctgccccaccagccgggggtgctggggatgtcagggaggcatggcgggcgggcagagccctgtgggttttgcatgtggctgaaaagcctggtctaggctgtggtgggaggagaaagaccgagtagggcatgggggtgggtgtgcaaggggggtgtgtccggtgtgtgtgtgtggtgtatgttatgtgtgtggtgtgtgtccatatgtgtaatgtgtgttgtgtgtcaacgtgtgtttgtcacgtgtggtgtgtgttgtgtgatatgtgatgtgtgtctgggtgtgtgtggtatgtgtccgtgtgtgtgatgtgtgtctgagtggtattgtgtgtggtatgtggtgtctgtgtgttgtgtgtggtgtgtgtccgtgtgtggtgtgtgtggtgtctgttgtgtgtgtggtgtgttgtgtgtggtgtgtgtgtctgtgtgctgtgtgttgtgtgtctgggtgtgttgtgtgtccgtgtgtggtgcgtgttgtgtctgtgtgtggtgtgtgttgcgtatgttgtgtgtccgtgtgtgtgtggtgtgtgtctgtgtgttgtgtggtgtgtctgtggtgtgtctgtgtgttgtgtgtccgtgtgtggtacgtgttgtgtctgtgtggtgtgtgttgcatatgttgtgtgtctgtgtgtgctgtgtgtctgtgtagtgtgttgtgtggtctgtgtggtgtgtctgtgtgttgtgtgtccatgtgtggtacgtgttgtgtgttgtgtatgttgtgtgtcgatgtgtgtgttgtgtgtctgggtgtgtggtgtgtatgttatgtgtccgtgcacgtggtgcacgtgtgcatacgcctgcatgtgttcatttctgtgcgcatgtgttgtgtgtgtgtccgggtgtgtccatttccgtgtgcctgtatggggccatgtgttatgccgagggctgaccatgggggggtcctgggctgaccaggtgaggggtgagccccaccgccacccccttccacacctgggtgagggcagcagcagtgagggccctacccacccctcggtctcctccagtcccccaaggccctgatggggctaagtccatgcctccccagccctggccttgcagccgctgctgacccgccaggcaggcacgggtccttaaggttcggcacctgcatgggtgccctgggcactgccctgactgccctgcaggacacactgggtcctgggtgccaaggctaggcaggccctatgacacggtgaccacaggctcagctggaggagcgtttgctgatgcacaggtggttaagggaagaacgggccaccttgggtcagctgtgcgtgaccccgagcaacgtggctgtgggtggagtgggtgccctcgagtcccccagcccgaggggggccccagagcagacccagccacctctgtgcagctgtgctgagggctcctgtctcctgccccaggtatgtgaagggctaccctcccaactcgccctacatcggcagctccccaaccctgtgccacctcctgcctgtgaaagcccccttctgctgcctgcggctggacaaggtaaggctggcggctctgccgcgctctgcacccccagacgccagcaccgggccgtgcatacctgccctggtttctctttggtcactttacatttcgataccattgcaaacttctagcacagctgcaagaattctgcaagggagagccacagatccttcacccagattcagcagacggtcccttcctgtcccgtttgggctctccctctcacagtgtctatgttagaggcacctgttttctgagccatcttgagaacaggttggagccaccacacccctttcccctaatacatctacatgcgttcccaaagatcaagaaccatctcgtaactacagctcattaggacagggaccccggcccggcatgggtgtctgatccacagtccaagttcagattgggccgagggccccgaaggccttcagcgtcctgcgtggcagtgttttccccaccgggagcctgtcccggctgcgtcttagcaactcctttctcgttactctcccttaatctggaatgttccctggggtctcttgaccttaatatttttgcagagcagggaccagtgacctggtggcaggtgggtctgcgatgtttcttggtggtgggactctgcctgcatgtggggccagccctccctggaaactcgggggtcaattgtcaggtcactggcgatgtttacttggatctcatggcaagctggggtcagtagggtttccccactgtgaaaatgactgttttccttctggagttagcagcttctctgtgggggacgtgttgagatcaggaaagcaaaccactcgacccagcggcttcaacatccctcagtgggttcttttttgtttatttgttgtttggttttttttaagacggggtcccactctgtcatccaggctggagtgcagtggtgggatcatagctcactacagcctccacctcccaggctcaaggggtcctcccacctcagcctcccaagtggctgggaacacaggcacgcaccaccacacctggctaattttatttatttatttattttttgtagtgatggggtctcactatgttgcccaggctagtctcgaactcctggactcaagtgatcctcccacctcggcctcccaaagtgctggggttacaggcgtgagccaccatgcctgccatctgtggatggtttccagttccggaatctctctccagctgtgtgttagggctcggccacggaggggacctccccttccccatgtgtgtgtgtccttcacggaccacaggctcccagcatattctgtgggttataatctgtggccggtcattgcagtagcatcatccttgacagatcgttattgattctgatgctcggaccccctcagttcactgggtggggtgggggccttcaagcggcttctgggtgcttctgacacgatcccctcaccctttgagctcatctgtgctttccaaccggagatgttccagccacatcttggccttcgcctgctccagtctggaatgaaccacttctccaaggaaccctgattccttttagtggaaaatgatgattggaaaccaagacctgggcacgtggcgtgctcactgctgttggcatccctgctctgagaaaacgtgtgtgcatacacatgacacgtatctatgcccacacctagatctctagccacatgccaaaaccacatgttcacatggacaccgccctttccacccctgacatgactgctctgtgccgcctgtgggtacagctgtggcatctggtggtgcctgtagctccccacaggcgtgtgcaggccgtgaaagggctgtgggccgagaggctaacggctgggtgttcacgggggatgtttggtgaggggtctgggagggctccagtggccagcaggaaccacagccctgactccagccctgactccagcatctgcccccagggctgcaagcacaacagctatgaagacgccaaggcctacgggttcaagaacaagctgatcatcgtctcggcagagacggccggcaatgggctgtacaacttcatcgtgccactgcgggcctactacagatcccgcaaggagctgaaccccatcgtgctgctgctggacaacaagtgaggctcctggggctcagcccaccccgcccacccgggccctcagacctgcagccagcagcctccccaactgggcccacccttcgcctttgcagagggcacgggaacatggggcctctggcctggtcctctcagctttcctaaaaagggggactctccttcctgctcccaactcctcctgctcccagctcctccccacacccactcctgctcccagctcctccccacccccagctcctctggctcctgggtcctccctgctccctgttcccccagttcccagctccacccaactcccagctcctccttgcttccagctcccccagttcccagctactctccattcccagctcctccccactcccagcttctccccactcccagttccccctcactcccagctcctccctgctcccagttcctctggccaccagctcctccccactcccagttcctctgtctcccagctcctccatgctctcagctcctgtggctcccagctcctcccactctggtcctctctccccctttccccctcctcccttgtcactccttctgtcctgtttgcctcctgctccactcactctgagccccaggattggggtggagggataaatggctcttcctcctgggcacctttttgcccaggggaccctaggaccctgacagctgagcccagggtcatcttggctgtgtgacctcagcaggtcccaccctcccgggccttggtttccccttgaataaaataaagaatggcccactggccttaaagtactccccaggtcccatacgctgcggttctggggaacccctgcctggcccagctctgtgcatggagggtagggccccactgggcctgaggagggcaggccttgaagcagggtgggcccctccaggaccgctgtccccacaggcccgaccaccacttcctggaagccatctgctgcttccccatggtctactacatggagggctctgtggacaagtaaggcgtggccggccgaggctcgtgggggctccacacccacccctcccctcctcttccaaagtctggggtgaccccgaccgcaggtggggtggggggctgaggtcctcctgccttctgaccaaatcccggggtcctgtgggtggggagtgggccgcatcctcagccacgggccctcggtcccgccaccagcctggacagcctgctgcagtgtggcatcatctatgcggacaacctggtggtggtggacaaggagagcaccatgagcgccgaggaggactacatggcggacgccaagaccatcgtcaacgtgcagaccatgttccggtgcgtccagtgtccggggctcggctctaaaccaccccacagccacgaccacgggccctcgccctgagacccccacagccacgaccacgggccctcgccctgagacccccacagccacgaccacgggccctcgccctgagaccgccacagccacgaccacgggccctcgccctgagacccccacagctatgaccacaagcccccaccctgaaacccccacaaccacagccatgggaccacaccctgagacccccacagccatggactctgcccagagacccccacagccacgaccacaagccctccaccctgagagtcccacagccatgaccatgggcctctgccctgagaccccccacagccgtgggaccctgccctgagacccccatagccaagacagtgggccctgccctgagacccccccacagccatgggaccccgccctgagacccccacagccacatgatcatgggcccccaccctgagacctcctacaaccaccatgggccccgccctgagccgcctgcctcccccaggctcttccccagcctcagcatcaccacggagctcacccacccttccaacatgcgcttcatgcagttccgcgccaaggacagctactctctggctctttccaaactagaaaaggtgagcagccctgccccgtgccagctgccaccccagaatcccagaaagagtgggagaaaggggctcaggggaaagggggccagtgccatgggaggctgggctcctgccgccctcctgctggggaactcaggagatggcgtggggggcccagcatggacagggtgctcttgatggtggaaccaggagatggaggcagggcgtttccctgaccgcgtgtgaggcactgggaatgtggcccatagcagccttccatctccctgagcagggaccaggcctggccgtatctggaggccaaggccatctgtcctggcatggtcagttggtcagaactcccgtggggagccctcagatcggtggttccacataggttggccagtagctcttagtacagataacgcacacggctgcaccattccagacttctccgctgccctggccactacgcccgaccttgaaacaggttcagtataaccactgcctccttgtattgacaagggacggagggcctgagagggaaggggcctgccccggatcgcacagtggagcaagtggctgagctggaattggctttctgcccttggagctccatgaagggcaccacccagggtcaggtgtggaacccaggggcacgggcactgttgctgttgccttgatatctggctgaccccagccccaccgcattccccacctgctcaagctgggacagcaccaccttgtccacacgggggctggtgcccaggcagccctaggctgagagcagggagcaggcaccttggtcagcagcaatgctgtctgctctacacgaaccccgagctcaccggtctgggctgaagtcctcatcggcgagctgctgggacctgcgtggctgcaggattgtgacaccgtggaggatctactgagccaggcccggcctggccaccaacaccaggcagaggacacacacgggcactccctgcgacacggacatggctttgtcccacctgaacccgatggagagtgaaagtgtgttccggccagccgtggaccaagagcccatagggatcctggagtcagttctgccccagagaagcgagaggaggccgggctgggcagccaccaggtcaacaggggcttctgccttagagatgccagcctgaggcgtagggggatgctcgggcacctggtctcagcacaggagagggctctgtctgccctgcccgccgcccaagcccaccctggagcctgccctgcccagcgtcgggagctcctggccccagtccccggctcagccggcagaggggtgtgctctgcagctggaaccacggaaggggaggaagttcacaggattctgtgttcgggtgccgggccgctccccagggctgggaggactctccggatctggaagaccaagtctgaccctgtgtggacagggatgctgccgtggagtcggggtcgaggcagtcatcctggcgcggctcctccggcctcagccttgcacctctctgtcccacaaggtggcttgaagtttgggtcagcaagcacgcagccaacacgctcctgcccgccttcccgccaggcagccatcgccaatcacccacgctccgccccgccgtggggcccatctctttccccggcttcacctccactggggctctgtgttgccggggcgggtgcggccagctctccatctcagagcagtgaacagtcctgggctccagctccagtcatgcgattccgtggcagttgtgtgacagggaccaaggagaaatcaggacatcggcaaagcctctggaagcagccgtgagctcctctgggctgacatggtgctggggtccatgtgggacagatgccctgggccctgggcagggccaggcatatacagagacgtggagccgagggaggagagggtgccgtggaccacctgacccatcaccctctgcagaggctgggatccgcctccccagcatgcaaggagcctgtagaaggcaccaggggctgcgtcttctccagcctgggtggccttggccccagatcccccatgggtctctctgacgggccagcatacggtgacctgcttccaacaaggacacctgtggcagcttcagatccttatttaaaagacattaatgccaatttattataaaaatgtacatgtttagtatgtattttacttcaaagaatactgaaagtaaaaaaaaaaaaaaattgaagaaaagttaccccaaatccctgcagccaacatcttctacatccaggcccagtggaaggataaagacaggattccaggcacgactccggaaggaaaaagggaggaggtggagatggcatacacgaccgtcagtgagatgctctgccaggtgcagcggctcatgcctgtaatcccagcacttttagaggctgaagcaggaagatcgcttgaggccagaagttcgagaccagcccaggcaacatagcaagtccccatctatacaaaaaatttaatggctgggggcggtggctcatgcctgtaatcccagcactttgggaggctaaggcggacggatcacctgaggtcaggagttcgagaccaggctggccaacatggagaaacctctctactaaagacacaaaaattagccgggcgtggtggctgtaaccccagctacttgggaggctagggcaggagaattgtttgaacccgggatgcagaggttgcagtgagccgagattgtgccactgcactccaggctgggcgacagagcgagaccctgtctcaaaaaaaaccaagcaaaaaaatgaaactattattaaggatgctgctggtggggtgacatgcatctccccttgagggctggctccagaaggctctcagagccccggtgtgtgagaggagtctctcccctgaggcccacctcccctgccgtccacctcccctggtctccagcacccaggactcctccaccacttcattgctttcacatggtcagcctgtgtgatctctcctcctcaccaggagccttccttcaggctggctctgcccatcctggggctgcacggtgcctgtcctgctccctctgaccgggggtctcgtgctcaggaagggctcatccgctcctgtgtgctcctgtcttcactgtcacttgtgaatggcacccgctcagcccctgctgaagccctcctgtccagaaagcaggaggaaatctctggaaggtcctgaaacgtccagctttttctttccttccacgtctctctctggccctgtcgtggttcttgtttcctgtttagcgttgttctgagtaaaggaaaattaaaattgcaagttgttctggaatttcacgttcgtctccgcgaagtacggctggaattctggatgcaaatcaccacgattgcaaactcacacgcccatcgcagctcatcgcacctggttcttgccccaagagcggaaacgccgcacgaaactaattccactgtttctcacgccccagccttcccaccggccactgatgagtaagggcgaacccggaggaaaaggggcttctgcctgcttgtcctgtcgctgtcgtgtcttttacctgtgaatcgagttctggttccggggggaagtgtggcctccgtgggcgcacacgcctgcctgtgcacacttgcttggaggccgagctccagggttggggccctccgctgaagaatgacccgagagttggccacggccgggtccggagcccgtggatgtaccgcctcacgtgacagaagggcctctgcagatgggattaagtcacgggcctcgagatgggagatcctcctggagcatccggcaggttcacgccgtcagggtccttctagggagaggccagaggtccccatccggtgggaagaagctgcacggccagctcagaagagggaagagggagccacaagccaaggcaggtggcctccaggagctggaaaaggcaagagaaggaggcccccaggcctccagagggagcggggccctgcccaggcctggatccagcctgagacacctcaaactcccgactccagaaacgggagagaggagacaggtgtcgttttagctgctaagtttgcggtcatttgttacgggagccccaggaaactcagtgggtccattgcaagcctggagcacagggacggggaggccaaggacagcagggagcccgatgcttgctcctcctcagcccagactccacacgccctccggcaccacttacaaaacaccgggggaggagaggattatcaggaggatttggaatttcgggatggcaagagcagggcgcttgaccaagcgcagtgcccatggcagccgccgggagtccaccttctcatcagagccccacatcactcctcccgggctctggccctgagtgtgtctgggaagatagagctggttcaggcctgccggtgtatgcacacatgcagacacacactgtgtacacgtggacacacacaccatgcacaaatgtgcacacaggtatcatgcacacatgtacggtgcacacacagtgccctcgaccccgtggctgccgtgccactggagggacctcggcaccagcccatctgaggcccctcctttcccacagagggagcgagagaatggctccaacctggccttcatgttccgcctgccgttcgccgccggccgcgtcttcagcatcagcatgttggacacactgctctaccaggtcagcggggaagcggcagcaggagggtggcgcctgggtgggacccccgtcatgccctcagctcttcagcctggtccctgttctgaatgataggactccctctgaatgaccttccctggattccaaggagacctctgggccctgtccttgccccagggatttctgggccctttttgacctatgggtgcctggcaagggagttttcttagaaaaggcctcccagaactctgcctgtgggtcatgtggtgcttggggacctggtggttctgtgtgtgtgtatgcatgaggggtggcgagcccgtggccggtggggtatggacctgtgtcccacgcccgtgcccgcgtgcctcactgtggctccctccctccctccctccctccctccctccctccctccctccctggccagtccttcgtgaaggactacatgatcaccatcacccggctgctgctgggcctggacaccacgccgggctcggggtacctctgtgccgtaagtgcccctggctgcgctgggctgggggcgtgctgggctgtccaagtgggtggatgggcacctgcccctgattcacggtggccaggaggctctgggtgctgccacctgccccagccaactcagggttcccaccctgcagatgaaaatcaccgagggcgacctgtggatccgcacgtacggccgcctcttccagaagctctgctcctccagcgccgagatccccattggcatctaccggacagagagccacgtcttctccacctcggaggttctggggcagcctgggggctgggactgtggcagcccctgtcctgtgtgacccacagcatccccaccttccgggggctgggactgtggcagcccctgtcctgtgtgacccacagcatccccaccttcagtgtcagggacctgggctagatcagctttgctattgctggcagctccttccgtctggtccgtgtgcccacgtgtagcgcttccagctggagcagccatgacccctaccgggggcagagaggctcaggggagtcttcaggaagaatacgggcagcccctgtgctgcagtccacacagcagcaggcaccgtgcccaccagccctaagcatgttccgtgcagaccccaggctgaggcggcgtggggggcaggggtgcgcccacaggtcccagactgcgcctgtttcctttgcagccccacgacctcagagcccaggtaagcaacccctccgtgcccacgcagcttctgcggagcaccagaacatcgcagcttcacatggagaagcctgccaggccccacccacccacccacccacagggccctgggaaagccgaggcaggagcgggtcccacggatgtgtcggccccagcacggctcagagaaggctgttgacactcactccctcctgcactggtggtggggaagctgaggcctggggggagtagcctgtgagtttggacaccaggtgcacccccgccccctcccaggccccaccaaggcaggcagcccccatgcccatctggcctgaccagcccccagcctcccaggcctttctgaaggtgccacccaggtgggatgcctacctcttcccagcaccccacgacccacaggctctggcaccccacgttccccaggttcggggcctccaaagggtgtgtttgcaagagggatcagggctcatctgcagatggagaaattgacacgcagagggggtgacctctcagggcaaggccaggatgtgctggggtccagatgggtcccaggccggacccacagaaataagggctcagagaggcgaagggagctgccctcagcagcctggcgaatcccttgaccaggcccgggcagggtgggtggcccggcgatctgtgctcaggaggaaaccaggcacattctttccagaggaggaaggcacatgcaccctggggtctgtcgtcgtcctcttccctaacacccccagctgctcccacgcaggccccaagctgcagagcccacacctcttcctaagcccctgccccaaagcccgcgaccctcccggcagcctcacccctccccgccctgccctgccctgccctgcccagtcccagatctcggtgaacgtggaggactgtgaggacacacgggaagtgaaggggccctggggctcccgcgctggcaccggaggcagctcccagggccgccacacgggcggcggtgaccccgcagagcacccactgctacggcgcaagagcctgcagtgggcccggaggctgagccgcaaggcgcccaagcaggcaggccgggcggcggccgcggagtggatcagccagcagcgcctcagcctgtaccggcgctctgagcgccaggagctctccgagctggtgaagaaccgcatgaagcacctggggctgcccaccaccggctacggtaagggcacacggcgcgggtgggggccggacggacggactgggccagggtcgggccacagccaagaacagtcggccacggcgtcccggggcccgggccgtcctcctgtctgtcatctgtctgtctgtcagcctttctggccaccctctgcctgcctctactgtggggccagctgtgggcaccaccacggcccagcagaggccccgtgcagaggagggaagtggccatggcccaccctggtgctgggaggccacaggacccttgggcatccctggtgtgagccccgtagggaggtgctcgcttccactgctctggggcctcagtttccctctctgccaaattagagacaggagctggctgagccctttggccaggggaggcagctgtggggtggagagggccaggctgggctccccaagtcgagggcatcagctagagttgggccagccagctgcagccaggcctgctggacagcggatacccagggtccctgagttctcgcagagaacaagactggtggggaccccctcccagagcccccagccctcccagcagccagggtaggggggagactttgggggaacagttgggggaaaggcccatgccaggacatgcctctgtccaccatgggcctgttgccactcccacggcacagccccctcctgccgcttcagggagcccggcgtccgtcttcagcaacagatccgtgcagccccctctctctgcgccttgtggtgggaagtgtgcttgggcttcggggccctacatgccctgcctggcgtggagagaagctcatcccctccctcactctagagatgctacctccgttgatgaagccatgggccccagctccccagcaccgggccgctacccagtgacacccacccctgagccgggtcgaggctccaccttccctttgttccatctcagggacttgcctgatgctcagggctgcctctgtcccgtcggagtgccccgtctcccaaccctgcttcctgcgtctgtcacctgggtcggggtcttgagtcccctgagcagagtcaggagctcacggctggcccaggagctgctcacgggtgactgggctgggctggcctgcgggcccgaaggagactcagaggccagggtttggggaacaggtggcctgtgaagacctttcccggcccccagccctgtgggcgctggggaaggtgtccccctgtccccgccttgaagcttccctccatccccacagtgtccctgtcccgggagggacttcacgccctttcaggtggggctcacgaggctgaggggggtgcccagcattcatgtttgtggtcccctgtgactgctggcagaggatgatcccgcccggtctggccacccccagagctggtgcctgcctctgcgaccgctgcctgcccccagagcttggcaaggaaggagcaactgcctttctggtgaccgactcgcttctggtggccggcctcccgtgcagctgcccctgcacccgcccactgtgccggccgcccgcacctcgcttgctgatattctctctctctctctttctgtctgtgcctctttctgtgtgttctgtagaggacgtagcaaatttaacagccagtgatgtcatgaatcgggtaaacctgggatatttgcaaggtaattctgccctggcgggtgccgaccaccgcacaacggggctcgccaacccttcaccgccggcagccttgctgcgccatcccggccaggcaggtggacaggagctgtgagagcacgtcccaggtgtcagcccaggcggccctgttgggcaaggcctcgcatgccgacagagtgtcccctggctgggcgttgaggacgggcctgaggctgaggtctggggcttgtgctagtcaaggtgctgttggccgccaggcagagtggcgactgcccagctctcgggctcaccctggtgctgcttctcctgctttgctgtttacgatgagctctgatgtgaccggtgggccaccctggcctacagccagcccttccctgtggccggccccacaagcagtctgaggaccctaagctcctctgctctcaggaagcacccagagggtcccgagccagggccccgggaggagctcggtgaagcctgggctggatgcaggggtggggactccaagccacgaggagtaggcaggctttatggctgcccaggccaggtggatggtgaccttcgtcccccagctggggctccacgaggggattcttcctgctcggctcgagcagagccgctccacaccccgtttcctccaagaacatgctgtgccccgtcagtgctcagcagagtccgggccagggtgctcccaggggtcctggcagcctcaggggagctcagagcctgcccccgctgccacacctccctacagggtaacccaggagcttgaagggcctcctgagccaggacgggtgttagctcgggagggacctgtttgccacaatgcccttgcacccggaggcagggccaggcctggggcagccacagaagtaaagcgtcggcctctcccggctctgtgcttttccccacgatggcgctcatggaagaccctcgctctgccgggcactgtggtgctcggacctgcagctagcggccttcatgagtgggatgcctgcgtcgcccgctcccctcacccgccaaagcttcctgcggctcccactgcagctccagggcaggcatggccacagcgggcttggggggcgagggctcacaggactcccgcggcaggcatttcctgggctcggcctacatgcccgggagggtgacggtgccggggttctggggctggggagctgcacccccatgtttagattcaagcggagcctaggccaagaggcagaacaggaagagtcacaccctttgcggggagggcgggcagcatcctggggcccctccagcgtgcagcccccatcccacacacacagcgggaggaggcaccccagccccgatactcacacaaatcccctcttcacgccttcctcacagctgccgtggggggaggcactagttatccccagtttatagagaaggaaactgaggctcgggtggcggtagctctggcctggcgtgggggattcaggcgggagtgaccgtggatgccaagagccaggaggtctggaagcctctggggcctgcagggttaggaagcgggttggggggcgagcagagctaccactgaggcctggagtaaaggtcggagggtctgggtggaggagtcaggtagccctggggaccaggggcgaggtggccctgccccaggacatgcaccagaagagccaaagccagcccctgccccaccccaggccctcaggcacacctgctgccagccttcaccaagcaccgtggacacgcgaagctagtcctctgcgaccatcctgggcagccaggaccagtgccatgtgactgaccacaacccaggccctggcactgcagctggggcctggcatccccacccgccccctagcccagccccaagaaacacaaagcccagcctggctcagcccagtgagctctgcttacttcagaagcgtgtggtccgggcaccccagcgggagaagctggggcctgggggccagtctgtgcctgccggagtgaaatgagctggtggccccttgttctcggtgtcccactgggtgctcgtcacaaagccagtcaggcaggggggcagggtcccaagaacctactagagctgccctcaccacccactccctgtgccacctgccatgcggccaccccagaagcgtgtgtgggcacgagggtggggagggtgtgtctccctgcacagcctcagcatgaggcccctgggcctcctccaaaggcctggggaagaccccaccatgggttttgggaaaagagcaaaacagctgtgaggggcaggtgtcaggccgtcccatcccgcaggtgtggacacaggagctggcaccaaatgacctgccgccgtgtgcgaaaggcctgaagcagagctggtctcctggccggagctgccctggtcgcccccaagtcctagcctgtgcccccgccaccctggcagcagctgtgctcacactgtgggaactccaaagcccccactgcagagggaccagcatggcccccacctggctggccagcagcctccccgacttttcccttctcagggaccagggcagggctaccccaaggggagaggacgggcagagaggagggaatggcagcagccagggtggaggctccctggggtcccccgtcctgggtcacctttgttctctgtccttggagtcctggaacaggaatccaggagcttcccgtagacaccatcagatggataaactgaggccacagaccctttccagccagtgaacccagggccactgctgggatggtggcctctccctccctgtgcttggcagggctaccctgccggccccatggagactgtgagatgatgggggggctgtctctccaggaccgttctcaccctgtgggacacatgcccacgcccaccccatgagccggacgccaggctgtctcccatgaggggcagaggccatgttctcggagggatgagctgcggggtgggggtgctctgggctgatgccactcaggacttcagtctgaagcaaagggctgagcacagcgagtgggaatggcctgtccaggcccggggacagcggcagctgcagggccctgagggagggacagggctgagcagcctctggaggcgggggtgggggtgtgtggatgtggaggacggcatgcaggtgtgtatatgagtgagtgtgcaggggtctgtggataccataggcatgggttgctatgaagatctgtgcagacacacaaatgtgtgtggaatacagaggtgaatctgcaggtgtgtgcaggtgtagatgaaggcatgcatgcatgtgaggtgaacagacacgtcccggtgtctgcaggctgacgtacgtggcaggtgtgcgctggtgtgtgcaggtgtgctcagtgcctctgctcagggccacccctttgctgcgatgccacctagcaccagaggtgggtgcgggtcagagctggctcccagctgccctcctggctgtcccctgccctgtgtggagatgggacaggtgtcggtcagggatgggccctggctgggctcaccaggatgaggtgctggagcagaatggtcaggaaggccggaaagactcagctcatcctggagcccccacacctctgggcccctgcagagctgggaggggcagggctgggggggtgacgtctgcccggctgtgtcctttgcagacgagatgaacgaccaccagaacaccctctcctacgtcctcatcaaccctccgcccgacacgaggctggagcccagtgacattgtgtgagtagcacctgtgggctgtgtggagaccccccctgagcaccaggtgggcactggggagatgaggccacaggcaccacagtggggccgctcagcagagggctgagcaggggctgcccggcccacatccactccagggtcctctgtgccctcccgcagctatctcatccgctccgaccccctggctcacgtggccagcagctcccagagccggaagagcagctgcagccacaagctgtcgtcctgcaaccccgagactcgcgacgagacacagctctgagccagccctgcacggagctcaggccaccaagcccggggtcctcaggaaggacgtggaggagcgtgtgaggacacggtggcactagcgtgaccctggggatggcacactctactcaccatggctcctgggactccaccctggaaaggagcccctcatgcggggggagggccagctcacccctgggcacctgcaggctagtgaggagagttttttaacctatttttacacgtcgatgcagtccacttctctttacacagatgtaccgcaactcgtgaccagggctggctgggagggcaacgcagggactggacgccctacagggccgagcccaggctgtgctggagggtggggctggggtgcatggggaggggagcagaacccagaacccaggagccccgcgtgggccacacccaactcagagccggcctgagcgttcacggccaggcagcctcgcttccttgcagccaagggctgggggccagggctgctgttctgcactctggggtgggtgagggggaccctgggctgtttgctgtcccaagccccttctggaagttagaagcagcaaagggcccggggaagccgggcatgtgagaggggtgcgtccccaggtcccccagagggccctgtcgccgaggacctttctgaaggaagcagaagacgccatttcctctacttcacactgaactgtcccagccactgcatctagggggcattgggcggaagatggtgcatttccatggaccattttacacttaccttttaaagcaaagcctcattttctaaacccctgacttgtgaagcacaattcagcctccgggctgggccacgtggagagagaggatcttctcagcaaggcgagatcccgggcggcggctgacatcaggagcgccaccctgcgtcctttgctgctggttccttactggtttgtacggtcagcgctggaaacttctattaaatggatgcattctggaggcatgaagttacaagtcaagtcgccctgctcgtgtttccaaggctctcacccctcccagccaccccactttaagggttacaaacacctgctggggtccccaccccaaccccataggcaagcccccattccccagccaggccaggacagtccttccaaaactcgggaaccaaattgtatttggctactggtgactggatcctggtagccaggaaacctgcctggtggtgggggtcccagagtccaggagggctgtctggtgagctgcccatcagcctcacccctgcagccaggcatgtccctggggtgggcacagagaccccaggctctgcccgcagtggcacagaactcatctgaggccagtggctgctggggatcccctacactgggggtcagggctgccccaggtggggatgtgtgtgcacctcaccacgttcacttcagggtaccccaagaggctgaaggggaaggaccaaaaggccgaggtgcagcccctccccggtgtcagggcagacaacacagcagctgctggaggggccggccctggccacacagactagctagtcccttactcccggcctgtctggaaccctcctgctcagaaggtgcccactagccctctgtgggggacagagccagacatgggtggtcagggagaggctgtgtggattcaggggaccagaaagtaagtcccaggaccttgatggagcggcagggattgatgttgggctagggtggccagagcctgtcccagcagggctggggtctatcacgttcctgggatccaagcagcgagcacgccctgccccgcagtcaccccgccccgcagtcgccctgcagctggaaggcccaagtctgcctcacctgggtggcctctcatgtcccccacaccctggcccccaggcgaggggggctgcacagcacctgcagggaggagaagggagagaaaagccggtctggctgctgggatgggagggccacagttccagcagtggcaggggaagctgtagcccctggagccccacactggaagagctggcctgcaggaggcaccatgggggagtcgcatgacttattcgggattgacttgcgatgtggatggtgttcccggagtcccctgtggccactccaccaccatgaggccgggaggcatcttagcctttgagcctctctccaggggtgagcggagccccccaaagagggctgaaggcttgctgcccaagaggggctgggtgagcacttggggcctctgagaacatcagtggtccgttccctcctgcacactggtggcaagtggcagcattttttcataatctccagtaatgaggccacttcgggtccagccctggacatccgaggaggaggcgggcagtccctgccccttcactaaccgcagaggatgccagctctaggccccctgctccgcctggagctcatgcgggcagccgtggacacaggtggcacccagcgcccagcggcctgtgaatcctcccgtgggcaaagctgggagccaggggctggaaccaggcaggtcagtgactgtgagatgccagctgccagcccaagaaaagctgcctgcagcatctggaaacttctgtgctctccttggcctctgtgttcttcatctccaggtttagggagcacccgggtgcctctctgcttgtcccgagcccactcaccaacagccccagcttgcacagtcatgacatcaggaaggtgggtccctgctcccagccgtcctcgtccaccatcacttctcccagcctcgtgtcctgctgacccataaaaggtccccctgcaaagtacaccaagtgaagtaggatctgagcaaaggttgagggactgaattccctaagaagtcatcactgcctagaataagcgaaaagaattttttttaatgttttacggtagaattatttgaaacatacaaaatgagtgagacacctgctattttccttattcctgttttttgtttgtttttattttccttatacctaattcatctaacagaaa

SEQ ID NO: 2 - DNA, 4782 bp, Homo sapiens
agggcaacgcgagggaagaaggtggcggctcccactcgcttctccctcgggtcgggtccgagctgccaggccgcatgccactccctgacggggcgcggaccccggggggcgtctgccgggaggcgcgcggcgggggctacaccaaccggaccttcgagtttgacgacggccaatgcgcccccaggcggccctgcgcgggggacggcgcgctcctggacaccgccggcttcaagatgagcgacctggactccgaggtgctgcccttgccgccgcgctaccgcttccgggacctgctgctgggcgacccgtccttccagaacgacgacagggtccaggtggagttctacgtcaacgagaacaccttcaaggagcggctcaagctgttcttcatcaaaaaccaaagatcgagcctgaggatccggctgttcaacttctccctgaagctgctcacctgcctgctctacattgtgcgcgtcctgctcgatgacccggccctgggcatcggatgctggggctgcccaaagcagaactactccttcaatgactcgtcctccgagatcaactgggctcctattctgtgggtggagagaaagatgacactgtgggcgatccaggtcatcgtggccataataagcttcctggagacgatgcttctcatctacctcagctacaaaggcaacatctgggagcagatcttccgcgtgtccttcgtcctggagatgatcaacactctgcccttcatcatcacgatcttctggccgccgctgcggaacctgttcatccccgtctttctgaactgctggctggccaagcacgcgctggaaaacatgattaatgacttccaccgtgccatcctgcggacacagtcagccatgttcaaccaggtcctcatcctcttctgcaccctgctgtgcctcgttttcacggggacctgcggcatccagcacctggagcgggcgggcgagaacctgtccctcctgacctccttctacttctgcatcgtcaccttctccaccgtgggctacggtgacgtcacgcccaagatctggccatcgcagctgctggtggtcatcatgatctgcgtggccctcgtggtgctcccactgcagttcgaggagctcgtctacctctggatggagcggcagaagtcagggggcaactacagccgccaccgtgcgcagacggagaagcacgtggtcctgtgtgtcagctccctcaagatcgaccttctcatggacttcctgaacgagttctacgcccacccccggctccaggactattacgtggtcatcctgtgccccacggagatggatgtccaggtgcgcagagtcctgcagatccctctgtggtcccagcgggtcatctacctccagggctctgcactcaaagaccaggacctcatgcgagccaagatggacaatggggaggcctgcttcatcctcagcagcaggaacgaggtggaccgcacggctgcagaccaccagaccatcctgcgcgcctgggccgtgaaggacttcgcccccaactgccccctctacgtccagatcctcaaacctgaaaacaagtttcacgtcaagtttgctgaccacgtggtgtgtgaggaggagtgcaagtacgccatgctggcgctgaactgcatctgcccggcgacctccaccctcatcaccctgctggtgcacacgtcccgcggccaggagggacaggagtctccggagcagtggcagcgcatgtatgggcgctgctccggcaacgaggtgtaccacatccgcatgggtgacagcaagttcttccgcgagtacgagggcaagagcttcacctacgcggccttccacgcccacaagaagtatggcgtgtgcctcatcgggctgaagcgggaggacaacaagagcatcctgctgaacccggggccccggcacatcctggccgcctctgacacctgcttctacatcaacatcaccaaggaggagaactcggccttcatcttcaagcaggaggagaagcggaagaagagggccttctcggggcaggggctgcacgagggtccggcccgcctgcccgtgcacagcatcatcgcctccatggggacagtggccatggacctgcagggcacagagcaccggcctacgcagagcggcggtgggggcgggggcagcaagctggcactgcccacggagaacggctcgggcagccggcggcccagcatcgcgcccgtcctggaactggccgacagctcagccctgctgccctgcgacctgctgagcgaccagtcggaggatgaggtgacgccgtcggacgacgaggggctctccgtggtagagtatgtgaagggctaccctcccaactcgccctacatcggcagctccccaaccctgtgccacctcctgcctgtgaaagcccccttctgctgcctgcggctggacaagggctgcaagcacaacagctatgaagacgccaaggcctacgggttcaagaacaagctgatcatcgtctcggcagagacggccggcaatgggctgtacaacttcatcgtgccactgcgggcctactacagatcccgcaaggagctgaaccccatcgtgctgctgctggacaacaagcccgaccaccacttcctggaagccatctgctgcttccccatggtctactacatggagggctctgtggacaacctggacagcctgctgcagtgtggcatcatctatgcggacaacctggtggtggtggacaaggagagcaccatgagcgccgaggaggactacatggcggacgccaagaccatcgtcaacgtgcagaccatgttccggctcttccccagcctcagcatcaccacggagctcacccacccttccaacatgcgcttcatgcagttccgcgccaaggacagctactctctggctctttccaaactagaaaagagggagcgagagaatggctccaacctggccttcatgttccgcctgccgttcgccgccggccgcgtcttcagcatcagcatgttggacacactgctctaccagtccttcgtgaaggactacatgatcaccatcacccggctgctgctgggcctggacaccacgccgggctcggggtacctctgtgccatgaaaatcaccgagggcgacctgtggatccgcacgtacggccgcctcttccagaagctctgctcctccagcgccgagatccccattggcatctaccggacagagagccacgtcttctccacctcggagccccacgacctcagagcccagtcccagatctcggtgaacgtggaggactgtgaggacacacgggaagtgaaggggccctggggctcccgcgctggcaccggaggcagctcccagggccgccacacgggcggcggtgaccccgcagagcacccactgctacggcgcaagagcctgcagtgggcccggaggctgagccgcaaggcgcccaagcaggcaggccgggcggcggccgcggagtggatcagccagcagcgcctcagcctgtaccggcgctctgagcgccaggagctctccgagctggtgaagaaccgcatgaagcacctggggctgcccaccaccggctacgacgagatgaacgaccaccagaacaccctctcctacgtcctcatcaaccctccgcccgacacgaggctggagcccagtgacattgtctatctcatccgctccgaccccctggctcacgtggccagcagctcccagagccggaagagcagctgcagccacaagctgtcgtcctgcaaccccgagactcgcgacgagacacagctctgagccagccctgcacggagctcaggccaccaagcccggggtcctcaggaaggacgtggaggagcgtgtgaggacacggtggcactagcgtgaccctggggatggcacactctactcaccatggctcctgggactccaccctggaaaggagcccctcatgcggggggagggccagctcacccctgggcacctgcaggctagtgaggagagttttttaacctatttttacacgtcgatgcagtccacttctctttacacagatgtaccgcaactcgtgaccagggctggctgggagggcaacgcagggactggacgccctacagggccgagcccaggctgtgctggagggtggggctggggtgcatggggaggggagcagaacccagaacccaggagccccgcgtgggccacacccaactcagagccggcctgagcgttcacggccaggcagcctcgcttccttgcagccaagggctgggggccagggctgctgttctgcactctggggtgggtgagggggaccctgggctgtttgctgtcccaagccccttctggaagttagaagcagcaaagggcccggggaagccgggcatgtgagaggggtgcgtccccaggtcccccagagggccctgtcgccgaggacctttctgaaggaagcagaagacgccatttcctctacttcacactgaactgtcccagccactgcatctagggggcattgggcggaagatggtgcatttccatggaccattttacacttaccttttaaagcaaagcctcattttctaaacccctgacttgtgaagcacaattcagcctccgggctgggccacgtggagagagaggatcttctcagcaaggcgagatcccgggcggcggctgacatcaggagcgccaccctgcgtcctttgctgctggttccttactggtttgtacggtcagcgctggaaacttctattaaatggatgcattctggaggcatgaagttaaaaaaaaaaaaaaa

SEQ ID NO: 3 - protein, 1235 aa, Homo sapiens
MPLPDGARTPGGVCREARGGGYTNRTFEFDDGQCAPRRPCAGDGALLDTAGFKMSDLDSEVLPLPPRYRFRDLLLGDPSFQNDDRVQVEFYVNENTFKERLKLFFIKNQRSSLRIRLFNFSLKLLTCLLYIVRVLLDDPALGIGCWGCPKQNYSFNDSSSEINWAPILWVERKMTLWAIQVIVAIISFLETMLLIYLSYKGNIWEQIFRVSFVLEMINTLPFIITIFWPPLRNLFIPVFLNCWLAKHALENMINDFHRAILRTQSAMFNQVLILFCTLLCLVFTGTCGIQHLERAGENLSLLTSFYFCIVTFSTVGYGDVTPKIWPSQLLVVIMICVALVVLPLQFEELVYLWMERQKSGGNYSRHRAQTEKHVVLCVSSLKIDLLMDFLNEFYAHPRLQDYYVVILCPTEMDVQVRRVLQIPLWSQRVIYLQGSALKDQDLMRAKMDNGEACFILSSRNEVDRTAADHQTILRAWAVKDFAPNCPLYVQILKPENKFHVKFADHVVCEEECKYAMLALNCICPATSTLITLLVHTSRGQEGQESPEQWQRMYGRCSGNEVYHIRMGDSKFFREYEGKSFTYAAFHAHKKYGVCLIGLKREDNKSILLNPGPRHILAASDTCFYINITKEENSAFIFKQEEKRKKRAFSGQGLHEGPARLPVHSIIASMGTVAMDLQGTEHRPTQSGGGGGGSKLALPTENGSGSRRPSIAPVLELADSSALLPCDLLSDQSEDEVTPSDDEGLSVVEYVKGYPPNSPYIGSSPTLCHLLPVKAPFCCLRLDKGCKHNSYEDAKAYGFKNKLIIVSAETAGNGLYNFIVPLRAYYRSRKELNPIVLLLDNKPDHHFLEAICCFPMVYYMEGSVDNLDSLLQCGIIYADNLVVVDKESTMSAEEDYMADAKTIVNVQTMFRLFPSLSITTELTHPSNMRFMQFRAKDSYSLALSKLEKRERENGSNLAFMFRLPFAAGRVFSISMLDTLLYQSFVKDYMITITRLLLGLDTTPGSGYLCAMKITEGDLWIRTYGRLFQKLCSSSAEIPIGIYRTESHVFSTSEPHDLRAQSQISVNVEDCEDTREVKGPWGSRAGTGGSSQGRHTGGGDPAEHPLLRRKSLQWARRLSRKAPKQAGRAAAAEWISQQRLSLYRRSERQELSELVKNRMKHLGLPTTGYDEMNDHQNTLSYVLINPPPDTRLEPSDIVYLIRSDPLAHVASSSQSRKSSCSHKLSSCNPETRDETQL

SEQ ID NO: 4 - DNA, 16 bp, Mus musculus
tgagaaagtcacgatg

SEQ ID NO: 5 - DNA, 16 bp, Mus musculus
gaggtggcataaagtc

SEQ ID NO: 6 - DNA, 16 bp, Homo sapiens
ggagaaggtgacgatg

SEQ ID NO: 7 - DNA, 16 bp, Homo sapiens
gaggtggcacagggtt

(19) United States
(12) Patent Application Publication
(10) Pub. No.: US 2020/0129538 A1
(43) Pub. Date: Apr. 30, 2020

(54) COMPOSITIONS AND METHODS FOR TREATING CONDITIONS ASSOCIATED WITH GAIN-OF-FUNCTION MUTATIONS IN KCNT1

(71) Applicant: The Florey Institute of Neuroscience and Mental Health, Melbourne, Victoria (AU)

(72) Inventor: Steven PETROU, Eltham, Victoria (AU)

(21) Appl. No.: 16/622,249

(22) PCT Filed: Jun. 13, 2018
(86) PCT No.: PCT/AU2018/050587

§ 371 Date: Dec. 12, 2019

(30) Foreign Application Priority Data
Jun. 13, 2017 (AU) 2017902242

(51) Int. Cl. A61K 31/711; C12N 15/113; A61P 25/08

(52) U.S. Cl. CPC A61K 31/711; C12N 15/1138; C12N 2310/11; C12N 2310/3231; C12N 2320/34; A61P 25/08

(57) ABSTRACT

Compositions and methods suitable for treating diseases and conditions associated excessive neuronal excitability, and/or diseases associated with gain-of-function mutations in KCNT1. More specifically, antisense oligonucleotides specific for KCNT1 and their use for treating diseases and conditions associated with excessive neuronal excitability and/or gain-of-function mutations of KCNT1.

### RELATED APPLICATIONS

This application claims priority to Australian Provisional Patent Application No. 2017902242, entitled “Compositions and Methods for Treating Conditions Associated with Gain-Of-Function Mutations in KCNT1” and filed on 13 Jun. 2017, the content of which is incorporated herein in its entirety.

### FIELD OF INVENTION

The present disclosure relates generally to compositions and methods suitable for treating diseases and conditions associated excessive neuronal excitability, and/or diseases associated with gain-of-function mutations in KCNT1. More specifically, the disclosure relates to antisense oligonucleotides specific for KCNT1 and their use for treating diseases and conditions associated with excessive neuronal excitability and/or gain-of-function mutations of KCNT1.

### BACKGROUND OF THE DISCLOSURE

KCNT1 encodes an intracellular sodium-activated potassium channel (potassium sodium-activated channel subfamily T member 1 that is expressed in the central nervous system. Also known as Slack, KCNT1 is a member of the Slo-type family of potassium channel genes and can co-assemble with other Slo channel subunits. These channels can mediate a sodium-sensitive potassium current (I_{KNa}), which is triggered by an influx of sodium channels ions through sodium channels or neurotransmitter receptors. It is thought that this delayed outward current is involved in regulating neuronal excitability.

Gain-of-function mutations in KCNT1 have been associated with particular forms of epilepsy, including epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome.

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including V271F, G288S, R428Q, R474Q, R474H, R474C, I760M, A934T and P924L (Barcia et al. (2012) Nat Genet. 44:1255-1260; Ishii et al. (2013) Gene 531:467-471; McTague et al. (2013) Brain. 136:1578-1591; Epi4K Consortium & Epilepsy Phenome/Genome Project. (2013) Nature 501:217-221; Lim et al. (2016) Neurogenetics; Ohba et al. (2015) Epilepsia 56:e121-e128). These mutations are gain-of-function, missense mutations that are dominant (i.e. present on only one allele) and result in change in function of the encoded potassium channel that causes a marked increase in whole cell current when tested inXenopus oocyte or mammalian expression systems (see e.g. Milligan et al. (2015) Ann Neurol. 75(4): 581-590; Barcia et al. (2012) Nat Genet. 44(11): 1255-1259; and Mikati et al. (2015) Ann Neurol. 78(6): 995-999).

ADNFLE has a later onset than EIMFS, generally in mid-childhood, and is generally a less severe condition. It is characterized by nocturnal frontal lobe seizures and can result in psychiatric, behavioural and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44:1188-1190). Functional studies of the mutated KCNT1 genes associated with ADNFLE indicated that the underlying mutations (M896I, R398Q, Y796H and R928C) were dominant, gain-of-function mutations (Milligan et al. (2015) Ann Neurol. 75(4): 581-590; Mikati et al. (2015) Ann Neurol. 78(6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including G652V and R474H, have been associated with West syndrome (Fukuoka et al. (2017) Brain Dev 39:80-83 and Ohba et al. (2015) Epilepsia 56:e121-e128). Treatment targeting the KCNT1 channel suggests that these mutations are gain-of-function mutations (Fukuoka et al. (2017) Brain Dev 39:80-83).

Quinidine is a small molecule drug that can block KCNT1 channels and which has been shown in both rodents and humans to have the potential to reverse the gain-of-function phenotype associated with the particular KCNT1 mutations (Milligan et al. (2015) Ann Neurol. 75(4): 581-590; Mikati et al. (2015) Ann Neurol. 78(6): 995-999; and Fukuoka et al. (2017) Brain Dev 39:80-83). However, there remains a need for additional compositions and methods for treating conditions that are associated with KCNT1 gain-of-function mutations as well as other conditions that are associated with excessive neuronal excitability.

### SUMMARY OF THE DISCLOSURE

The present disclosure relates generally to compositions and methods for treating diseases and conditions associated with excessive neuronal excitability and/or a gain-of-function mutation in KCNT1.

In one aspect, the present disclosure relates to an antisense oligonucleotide comprising a sequence of nucleobases that is complementary to a target region in KCNT1.

In some embodiments, the target region is within the KCNT1 sequence set forth in SEQ ID NO:1 or a variant thereof having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto. In a particular embodiment, the target region is within or spans all or a part of an exon, intron, an intron/exon junction, a 3′-untranslated region (UTR), a 5′-UTR, the translation initiation site and/or the translation termination site.

The antisense oligonucleotides of the present disclosure may hybridize to the pre-mRNA and/or mRNA of KCNT1. In some examples, the antisense oligonucleotide is an allele-specific oligonucleotide.

In one embodiment, the target region spans a nucleotide selected from among nucleotide 5236, 39323, 53882, 55173, 73279, 73631, 80231 or 91871 of SEQ ID NO:1. In a particular example, the antisense oligonucleotide specifically hybridizes to the pre-mRNA of a KCNT1 allele containing nucleotide(s) AC at position 5236, T at position 39323, C at position 55173, A at position 53882, G at position 73279, A at position 73631, A at position 80231, or C at position 91871.

The antisense oligonucleotides of the present disclosure may be, for example, 10 to 80, 10 to 60, 10 to 50, 10 to 40, 10 to 30 or 15 to 25 nucleobases in length.

In some embodiments, the antisense oligonucleotides of the present disclosure are at least 70%, 80%, 85%, 86%, 87%, 88%, 89%, 90%, 91%, 92%, 93%, 94%, 95%, 96%, 97%, 98%, or 99% complementary to the target region. In a particular embodiment, the antisense oligonucleotides comprise least 8, 9, 10, 11, 12, 13, 14, 15, 16, 17, 18, 19 or 20 contiguous nucleobases that are 100% complementary to the target region.

In some examples, the antisense oligonucleotides comprise at least one modification, such as, for example, a nucleobase modification, a modification of the oligonucleotide backbone or a modification of a ribose sugar. For example, the antisense oligonucleotides can comprise a modified sugar selected from among a 2-O-methyl (2OMe), 2-O-methoxy-ethyl (MOE), locked nucleic acids (LNA), 2-fluoro or S-constrained-ethyl (cEt). In some examples, the backbone of the antisense oligonucleotide comprises phosphorothioates.

In particular embodiments, the antisense oligonucleotides activate RNase H.

In a further aspect, the present disclosure relates to a composition comprising an antisense oligonucleotide described above and herein.

In another aspect, the present disclosure is directed to a method for treating a disease or condition associated with a gain-of-function mutation in KCNT1 in a subject, comprising administering to the subject an antisense oligonucleotide or composition described above and herein. In some embodiments, the disease or condition is selected from among epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome. In particular examples, the subject is confirmed as having a KCNT1 allele containing a gain-of-function mutation (e.g. V271F, G288S, R398Q, R428Q, R474Q, R474H, R474C, G652V, I760M, Y796H, M896I, P924L, R928C or A934T).

The methods for treating a disease or condition associated with a gain-of-function mutation in KCNT1 in a subject may comprise administering to the subject an allele-specific antisense oligonucleotide described above and herein. In a particular example, the subject has been genotyped to identify an allele-SNP that is associated with the gain-of-function mutation. Exemplary allele-specific SNPs include SNP 9:138594266 A/AC (rs5901089) at nucleotide (nt) 5236 of SEQ ID NO:1, SNP 9:138662309 A/G (rs10776844) at nt 73279, SNP 9:138669261 G/A (rs914428 at nt 80231, SNP 9:138662661 G/A (rs10858172) at nt 73631, SNP 9:138642912 C/A (rs10122976) at nt 53882, SNP 9:138644203 T/C (rs10735239) at nt 55173, SNP 9:138628353 C/T (rs7350168) at nt 39323, and SNP 9:138680901 T/C (rs10858173) at nt 91871.

The present disclosure is also directed to a method for treating a disease or condition selected from among epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome, comprising administering to a subject with the disease an antisense oligonucleotide or composition described above and herein.

In the methods of the present disclosure, the antisense oligonucleotide or composition may be administered to the subject by parenteral administration (e.g. subcutaneous administration, intravenous administration, intramuscular administration, intraarterial administration, intraperitoneal administration, or intracranial administration) or intranasal administration. In instances where administration is intracranial administration, it may be, for example, intrathecal or intracerebroventricular.

In some examples, the antisense oligonucleotide or composition is administered to the subject about every 1, 2, 3, 4, 5, 6, 7, 8, 9, 10, 11, 12 or more months. In a particular embodiment, the antisense oligonucleotide or composition is administered to the subject about every 3 months.

In a further aspect, the present disclosure is related to the use of an antisense oligonucleotide or composition described above and herein for the preparation of a medicament for treating a disease or condition associated with a gain-of-function mutation in KCNT1. In an additional aspect, the present disclosure is related to the use of an antisense oligonucleotide or composition described above and herein for the preparation of a medicament for treating a disease or condition selected from among epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome.

### BRIEF DESCRIPTION OF THE DRAWINGS

FIG. 1 shows KCNT1 mRNA expression in the brains of mice administered one of two LNA oligonucleotides specific for the KCNT1 mRNA (oligonucleotides LNA 5 and LNA 6) compared to the untreated controls (n=3). Expression levels are shown normalized to the expression levels in untreated mice (presented as a dashed line at 100%).

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

An “antisense oligonucleotide” refers to a single-stranded oligonucleotide having a sequence that permits hybridization to a corresponding region or segment of a target nucleic acid. Reference to an antisense oligonucleotide includes reference to both unmodified and modified antisense oligonucleotides, wherein a modified antisense oligonucleotide contains at least one modified nucleoside and/or modified internucleoside linkage.

“Complementary,” as used herein, refers to the capacity for precise pairing between two nucleobases, such as between a nucleobase in an antisense oligonucleotide and a nucleobase in a KCNT1 mRNA or pre-mRNA. The antisense oligonucleotide and the mRNA or pre-mRNA are complementary to each other when a sufficient number of corresponding positions in each molecule are occupied by nucleobases which can hydrogen bond with each other. Thus, “complementary” is used to indicate a sufficient degree of precise pairing over a sufficient number of nucleotides such that stable and specific binding occurs between the antisense oligonucleotide and the mRNA or pre-mRNA. It is understood that the antisense oligonucleotide need not be 100% complementary to the target region in the KCNT1 mRNA or pre-mRNA to hybridize thereto. Moreover, an oligonucleotide may be complementary to, and hybridize, over one or more segments such that intervening or adjacent segments are not involved in the hybridization event. “Complementary” as used herein therefore includes reference to less that 100% complementary, such at least or about 70%, 75%, 80%, 85%, 90% or 95% sequence complementarity.

As used herein, a “disease or condition associated with a gain-of-function mutation in KCNT1” refers to a disease or condition that is associated with, is partially or completely caused by, or has one or more symptoms that are partially or completely caused by, a mutation in KCNT1 that results in a gain-of-function phenotype, i.e. an increase in activity of the potassium channel encoded by KCNT1 resulting in an increase in whole cell current.

As used herein, “expression of KCNT1” refers to the transcription of mRNA from KCNT1 or the translation of protein from the KCNT1 mRNA. KCNT1 expression can be assessed using any method known in the art, including, but not limited to, Northern blot, Western blot and qRT-PCR.

As used herein, a “gain-of-function mutation” is a mutation in KCNT1 that results in an increase in activity of the potassium channel encoded by KCNT1. Activity can be assessed by, for example, ion flux assay or electrophysiology (e.g. using the whole cell patch clamp technique). Typically, a gain-of-function mutation results in an increase of at least or about 20%, 30%, 40%, 50%, 60%, 70%, 80%, 90%, 100%, 125%, 150%, 175%, 200%, 225%, 250%, 275%, 300%, 325%, 350%, 375%, 400% or more compared to the activity of a potassium channel encoded by a wild-type KCNT1.

A “gapmer” as referred to herein is a chimeric antisense oligonucleotide in which an internal region having a plurality of nucleotides that support RNase H cleavage is positioned between external regions having one or more nucleotides, wherein the nucleotides comprising the internal region are chemically distinct from the nucleoside or nucleotides comprising the external regions.

As used herein, “hybridization” means the pairing of substantially complementary strands of nucleic acids, such as between an antisense oligonucleotide of the disclosure and a KCNT1 mRNA or pre-mRNA. One mechanism of pairing involves hydrogen bonding, which may be Watson-Crick, Hoogsteen or reversed Hoogsteen hydrogen bonding, between complementary nucleobases of the strands of nucleic acids. For example, adenine and thymine or uracil are complementary nucleotides which pair through the formation of hydrogen bonds. Hybridization can occur under varying circumstances. Reference to “specifically hybridizes” as used herein means that the antisense oligonucleotide hybridizes to a target region in one KCNT1 allele, such as a mutant KCNT1 allele, and not to the same target region in another KCNT1 allele, such as a wild-type KCNT1 allele.

An “inhibition of expression of KCNT1” in the context of the present disclosure means that there has been a reduction in the level of expression of KCNT1 when cells expressing KCNT1 have been contacted with an antisense oligonucleotide specific for KCNT1, compared to the level of expression observed when the cells have not been contacted with the antisense oligonucleotide. Typically, expression levels of KCNT1 are reduced by at least or about 20%, 25%, 30%, 35%, 40%, 45%, 50%, 55%, 60%, 65%, 70%, 75%, 80%, 85%, 90% or more.

The terms “linked” and “attached” are used interchangeably and relate to any type of interaction that join two entities, such as an antisense oligonucleotide and a moiety (e.g. a cell penetrating peptide), and include covalent bonds or non-covalent bonds, such as, for example, hydrophobic/hydrophilic interactions, van der Waals forces, ionic bonds or hydrogen bonds.

As used herein, “nucleobase” means a heterocyclic moiety capable of pairing with a base of another nucleic acid, and includes, for example, adenine (A), guanine (G), cytosine (C), thymine (T) and uracil (U). Reference herein to nucleobase also includes reference to a modified nucleobase.

A “nucleoside” as used herein refers to a nucleobase linked to a sugar. Reference herein to a nucleoside also includes reference to a modified nucleoside, which has a modified sugar moiety or modified nucleobase. A “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

As used herein, “nucleotide” refers to a nucleoside having a phosphate group covalently linked to the sugar portion of the nucleoside. Reference herein to a nucleotide also includes reference to a modified nucleotide, which has a modified sugar moiety, modified internucleoside linkage, or modified nucleobase. A “nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

As used herein, the term “sequence identity” or “% identical” or grammatical variations means that in a comparison of two sequences over a specified region the two sequences have the specified number or percentage of identical residues in the same position. Sequences can be aligned by any method known to those of skill in the art. Such methods typically maximize matches, and include methods such as using manual alignments and by using the numerous alignment programs available.

As used herein the terms “treating” or “treatment” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of the present invention, symptoms that may be ameliorated, reversed, prevented, retarded or the linked include but are not limited to seizures and spasms.

The term “subject” as used herein refers to an animal, in particular a mammal and more particularly a primate including a lower primate and even more particularly, a human who can benefit from the protocol of the present invention. A subject regardless of whether a human or non-human animal or embryo may be referred to as an individual, subject, animal, patient, host or recipient.

### Antisense Oligonucleotides Specific for KCNT1

The present disclosure provides antisense oligonucleotides that are specific for KCNT1 and which function to inhibit expression of KCNT1 when cells are contacted with the antisense oligonucleotide. Typically, this is achieved either by preventing or inhibiting translation (e.g. by targeting the translation start site with the antisense oligonucleotide or sterically blocking the binding of RNA binding protein complexes, such as ribosomal subunits, with the antisense oligonucleotide) or by degrading the mRNA or pre-mRNA, a process mediated by RNase H upon generation of the DNA/RNA duplex formed between the antisense oligonucleotide and pre-mRNA or mRNA The resulting inhibition of KCNT1 expression is reflected in a reduction in the level of KCNT1 mRNA and/or protein. Typically, expression levels of KCNT1 are reduced by at least or about 20%, 25%, 30%, 35%, 40%, 45%, 50%, 55%, 60%, 65%, 70%, 75%, 80%, 85%, 90% or more. Accordingly, the antisense oligonucleotides of the present disclosure can be used for treating a disease or condition associated with a gain-of-function mutation in KCNT1.

The antisense oligonucleotides of the present disclosure are complementary to a target region within KCNT1, and can therefore hybridize to that target region within the KCNT1 pre-mRNA (i.e. the precursor mRNA containing both introns and exons) and/or KCNT1 mRNA. Exemplary human KCNT1 genes include those with a sequence set forth in SEQ ID NO:1 (NCBI Reference Sequence: NG_033070) and variants thereof, including variants containing single nucleotide polymorphisms, such as those described in the various publically available databases (e.g. the Genome Aggregation Database (gnomAD) (http://gnotnad.broadinstitute.org/), a selection of which are also set forth below. Typically, the KCNT1 variants have at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the sequence set forth in SEQ ID NO:1. The human KCNT1 gene set forth in SEQ ID NO:1 contains 31 exons that are interspersed between nucleotides (nt) 5001 and 95963: exon 1: nt 5075-5184; exon 2: nt 17393-17536; exon 3: 52914-52993; exon 4: nt 53758-53857; exon 5: nt 56753-56809; exon 6: nt 57937-57985; exon 7: nt 59689-59748; exon 8: nt 59972-60046; exon 9: nt 60114-60197; exon 10: nt 61230-61324; exon 11: nt 62495-62675; exon 12: nt 67847-68011; exon 13: nt 68440-68576; exon 14: nt 71426-71489; exon 15: nt 71645-71753; exon 16: nt 72763-72871; exon 17: nt 73114-73263; exon 18: nt 73673-73911; exon 19: nt 75531-75765; exon 20: nt 78126-78231; exon 21: nt 80154-80326; exon 22: nt 81240-81311; exon 23: nt 81504-81638; exon 24: nt 82175-82286; exon 25: nt 86840-86941; exon 26: nt 87351-87434; exon 27: nt 87577-87705; exon 28: nt 88126-88146; exon 29: nt 89013-89337; exon 30: nt 94613-94697; and exon 31: nt 94857-95963. This gene is transcribed into the KCNT1 mRNA represented by the cDNA sequence set forth in SEQ ID NO:2 (NCBI Reference Sequence: NM_020822.2), which is translated into the potassium channel subfamily T member 1 protein set forth in SEQ ID NO:3 (NCBI Reference Sequence: NP_065873.2).

In some examples, the target region to which the antisense oligonucleotide is complementary is within or spans all or a part of an exon such that the antisense oligonucleotide hybridizes to the KCNT1 pre-mRNA and mRNA. For example, in one embodiment, the oligonucleotide targets nucleotides 998-1013 of the KCNT1 mRNA set forth as cDNA in SEQ ID NO:2, and thus has a sequence of, for example, GGAGAAGGTGACGATG (SEQ ID NO:6). In a further embodiment, the oligonucleotide targets nucleotides 2366-2381 of the KCNT1 mRNA set forth as cDNA in SEQ ID NO:2, and thus has a sequence of, for example, GAGGTGGCACAGGGTT (SEQ ID NO:7). In other examples, the target region is within or spans all or a part of an untranslated region of KCNT1, such as an intron, an intron/exon junction, or a 3′- or 5′-untranslated region (UTR). In such examples, and in particular when the target region is within an intron or spans an intron/exon junction, the antisense oligonucleotide may only hybridize to the KCNT1 pre-mRNA. In some instances, the target region includes particular structural or functional sites, such as the translation initiation site (including start codon), the translation termination site (including stop codon).

In some embodiments, the antisense oligonucleotides of the present disclosure are complementary to a conserved target region of KCNT1 and therefore hybridize to all or essentially all alleles of KCNT1 within a population. Typically, such conserved target regions are with an exon, although they may be within or span an intron, an intron/exon junction, or a 3′- or 5′-UTR.

In other embodiments, the antisense oligonucleotides of the present disclosure are allele-specific antisense oligonucleotides. Allele-specific antisense oligonucleotides bind to a region of KCNT1 containing a SNP that is associated with (i.e. present within) the allele containing the gain-of-function mutation in KCNT1. Specific targeting of the disease-associated allele by the allele-specific antisense oligonucleotide can therefore result in inhibition of expression from this KCNT1 allele while leaving expression from the normal, non-disease-associated allele unchanged.

Databases describing SNPs within KCNT1 and their frequency are widely available, and include, for example, the Genome Aggregation Database (gnomAD) (http://gnomad.broadinstitute/org/). Such databases can be used to identify SNPs that are most commonly present in the population and which can therefore be targeted by allele-specific antisense oligonucleotides. The most common single nucleotide polymorphisms (SNPs) within KCNT1 include 9:138594266 A/AC (rs5901089) (wherein “9” indicates chromosome 9, “138594266” represents the position of the polymorphism on chromosome 9, “A” represents the replaced nucleotide, “AC” represents the replacing nucleotides, and “rs5901089” is the NCBI reference number of the SNP); 9:138662309 A/G (rs10776844); 9:138669261 G/A (rs914428); 9:138662661 G/A (rs10858172); 9:138642912 C/A (rs10122976); 9:138644203 T/C (rs10735239); 9:138628353 C/T (rs7350168); 9:138680901 T/C (rs10858173). The Table below indicates the allele frequency of these SNPs and the position within KCNT1 (as set forth in SEQ ID NO:1) that these SNPs occur.

TABLE 1

| | Allele | Position within KCNT1 |
|---|---|---|
| SNP | frequency | (SEQ ID NO: 1) |
| 9:138594266 A/AC (rs5901089) | 0.8995 | 5236 |
| 9:138662309 A/G (rs10776844) | 0.7105 | 73279 |
| 9:138669261 G/A (rs914428) | 0.6683 | 80231 |
| 9:138662661 G/A (rs10858172) | 0.4820 | 73631 |
| 9:138642912 C/A (rs10122976) | 0.4694 | 53882 |
| 9:138644203 T/C (rs10735239) | 0.4689 | 55173 |
| 9:138628353 C/T (rs7350168) | 0.3590 | 39323 |
| 9:138680901 T/C (rslO858173) | 0.3005 | 91871 |

Thus, amongst the antisense oligonucleotides of the present disclosure are those that are complementary to, and hybridize to, a target region spanning nucleotide 5236, 39323, 53882, 55173, 73279, 73631, 80231 or 91871 of SEQ ID NO:1, wherein the antisense oligonucleotides specifically bind to the pre-mRNA of an allele containing nucleotide(s) AC at position 5236, T at position 39323, C at position 55173, A at position 53882, G at position 73279, A at position 73631, A at position 80231, or C at position 91871.

The antisense oligonucleotides of the present disclosure are typically 10 to 80 nucleobases in length, such as 10 to 60, 10 to 50, 10 to 40, 10 to 30 or 15 to 25 nucleobases in length. Thus, in particular examples, the antisense oligonucleotides are 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

The antisense oligonucleotides may be 100% complementary across their entire length to a target region of KCNT1 or may be less than 100% complementary. Typically, the antisense oligonucleotides are at least 70%, 80%, 85%, 86%, 87%, 88%, 89%, 90%, 91%, 92%, 93%, 94%, 95%, 96%, 97%, 98%, or 99% complementary to a target region of KCNT1. The antisense oligonucleotides may contain, for example, at least 8, at least 9, at least 10, at least 11, at least 12, at least 13, at least 14, at least 15, at least 16, at least 17, at least 18, at least 19, or at least 20 contiguous nucleobases that are complementary to a target region in KCNT1. In instances where the antisense oligonucleotides are not 100% complementary, the mismatched or non-complementary nucleobase(s) can be clustered or interspersed with complementary nucleobases and need not be contiguous to each other. The non-complementary nucleobase(s) may be located at the 5′ end and/or 3′ end of the antisense compound. Alternatively, the non-complementary nucleobase(s) can be at an internal position of the antisense oligonucleotide. When two or more non-complementary nucleobases are present, they can be either contiguous or non-contiguous.

In particular embodiments, antisense oligonucleotides of the present disclosure are up to 10, 12, 13, 14, 15, 16, 17, 18, 19, 20, 21, 22, 23, 24, 25, 26, 27, 28, 29 or 30 nucleobases in length and comprise no more than 6, 5, 4, 3, 2, or 1 non-complementary nucleobase(s) relative to a target region in KCNT1.

The antisense oligonucleotides of the present disclosure can be produced using any method known in the art. Typically, the antisense oligonucleotides are produced using chemical synthesis methods. While the antisense oligonucleotides can be unmodified, more typically the antisense oligonucleotides of the present disclosure contain one or more modifications. These modifications can function to, for example, increase stability of the antisense oligonucleotide (e.g. increase resistance of the antisense oligonucleotide to degradation by nucleases), increase affinity of the antisense oligonucleotide to the target mRNA or pre-mRNA, increase steric hindrance by the antisense oligonucleotide, increase RNase H activity, and/or improve intracellular uptake. Exemplary modifications that are well known to those skilled in the art include, but are not limited to, modification of the nucleobase, modification of the backbone phosphate linkages (e.g. phosphodiester, phosphoramidate, or phosphorothioate (PS) modification), modifications of the ribose sugar (e.g. 2-O-methyl (2OMe), 2-O-methoxy-ethyl (MOE), locked nucleic acids (LNA), 2-fluoro and S-constrained-ethyl (cEt) modifications) and other modifications such as replacement of the entire sugar phosphate backbone with polyamide linkages to produce peptide nucleic acids (PNA) and the use of a morpholine ring instead of the ribose ring and phosphoroamidate intersubunit linkages to produce phosphorodiamidate morpholino oligomers (PMO) (broadly reviewed in, for example, Sardone et al. (2017) Molecules 22(4): 563 Evers et al. (2015) Adv Drug Del Rev 87:90-103; Kole et al. (2012) Nat Rev Drug Discov. 11(2): 125-140).

In particular embodiments, the antisense oligonucleotides of the present disclosure contain one or more modified nucleobases. These can function to, for example, increase stability or binding affinity of the antisense oligonucleotide. Exemplary modified nucleobases include, but are not limited to, N⁶-methyladenine, N²-methylguanine, hypoxanthine, 7-methylguanine, 5-methylcytosine, 5-hydroxymethylcytosine, pseudouracil, 4-thiouracil, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcyto sine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N⁴-ethylcytosine, or derivatives thereof; N²-cyclopentylguanine (cPent-G), N2-cyclopentyl-2-aminopurine (cPent-AP), and N²-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). In particular embodiments, the antisense oligonucleotides contain one or more modified nucleobases that increase the binding affinity of the antisense oligonucleotide to the KCNT1 mRNA or pre-mRNA, such as 5-methylcytosine (5-me-C), 5-substituted pyrimidines, 6-azapyrimidines and N-2, N-6 and 0-6 substituted purines, including 2 aminopropyladenine, 5-propynyluracil and 5-propynylcytosine.

The antisense oligonucleotides of the present disclosure may comprise modified sugar moieties. Exemplary sugar moiety modifications include 2-O-methyl (2OMe), 2-O-methoxy-ethyl (MOE), locked nucleic acids (LNA), 2-fluoro and S-constrained-ethyl (cEt) modifications.

In particular embodiments, the backbones of the antisense oligonucleotides of the present disclosure comprise phosphorothioates, chiral phosphorothioates, phosphorodithioates, phosphotriesters, aminoalkylphosphotriesters, methyl or other alkyl phosphonates comprising 3′alkylene phosphonates or chiral phosphonates, phosphinates, phosphoramidates comprising 3′-amino phosphoramidate and aminoalkylphosphoramidates, thionophosphoramidates, thionoalkylphosphonates, thionoalkylphosphotriesters, or boranophosphates. In other embodiments, the backbone has no phosphorus atom. Exemplary oligonucleotide backbones that do not include a phosphorus atom include those that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside; see e.g. owned U.S. Pat. Nos. 5,698,685, 5,217,866, 5,142,047, 5,034,506, 5,166,315, 5,185, 444, 5,521,063, 5,506,337, 8,076,476, 8,299,206 and 7,943,762); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In one example, the antisense oligonucleotides of the present disclosure are a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone (see e.g. U.S. Pat. Nos. 5,539,082; 5,714,331; and 5,719,262).

In particular embodiments, the antisense oligonucleotides of the present invention are partially or completely resistant to RNase H. Such antisense oligonucleotides can include 2′-O-methyl derivatives, and/or phosphorothioate backbones, both of which are resistant to nuclease degradation. In further examples, the antisense oligonucleotides do not activate RNase H, typically by virtue of the presence of one or more structural modifications that sterically hinders or prevent binding of RNase H to a duplex molecule containing the antisense oligonucleotide and the KCNT1 mRNA or pre-mRNA. For example, such antisense oligonucleotides include those where at least one, or all, of the inter-nucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl).

In other examples, the antisense oligonucleotides of the present disclosure activate RNase H when they form a DNA-RNA duplex with the KCNT1 mRNA or pre-mRNA. Exemplary of such antisense oligonucleotides are gapmers, which are chimeric molecules containing at least one region modified so as to confer increased resistance to nuclease degradation, increased cellular uptake, increased binding affinity for the target nucleic acid, and a second region that serves as a substrate for RNase H. Gapmers have an internal region having a plurality of nucleotides that support RNase H cleavage. This internal region is positioned between external regions having a plurality of nucleotides that are chemically distinct from the nucleosides of the internal region, and which serve to, for example, increase stability of the antisense oligonucleotide and protect it from nuclease degradation. In certain embodiments, the external regions of the gapmer contain -D-ribonucleosides, -D-deoxyribonucleosides, 2′-modified nucleosides (e.g. 2′-MOE, and 2′-O—CH₃, among others), bridged nucleic acids (BNAs), or locked nucleic acids (LNAs).

The antisense oligonucleotides of the present disclosure may also be linked to one or more one or more moieties that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, carbohydrates, phospholipids, biotin, phenazine, folate, phenanthridine, anthraquinone, acridine, fluoresceins, rhodamines, coumarins, and various dyes.

In particular embodiments, the antisense oligonucleotides are linked to a cell-penetrating peptide (CPP) that is effective to enhance transport of the compound into cells. The transport moiety can be attached to either terminus of the antisense oligonucleotide, resulting in increased penetration of the antisense oligonucleotides into cells and macromolecular translocation within multiple tissues in vivo upon systemic administration. In one embodiment, the cell-penetrating peptide is an arginine-rich peptide transporter. Antisense oligonucleotides linked with arginine-rich CPPs were able to cross the blood-brain barrier and were widely distributed throughout the brain of wild-type mice following systemic delivery (Du et al. Hum. Mol. Genet., 20 (2011), pp. 3151-3160). In another embodiment, the cell-penetrating peptide may be Penetratin or the Tat peptide. These peptides are well known in the art and are disclosed, for example, in US Publication No. 20100016215. The transport moieties described above have been shown to greatly enhance cell entry of attached oligomers, relative to uptake of the oligomer in the absence of the attached transport moiety. For example, antisense oligonucleotides linked with arginine-rich CPPs were able to cross the blood-brain barrier and were widely distributed throughout the brain of wild-type mice following systemic delivery (Du et al. Hum. Mol. Genet., 20 (2011), pp. 3151-3160). Uptake may be enhanced at least ten-fold, or at least twenty-fold, relative to the unconjugated compound.

The antisense oligonucleotides can also be modified to have one or more stabilizing groups that are generally attached to one or both termini to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps.

### Assessment of the Antisense Oligonucleotides

The activity of the antisense oligonucleotides of the present disclosure can be assessed and confirmed using various techniques known in the art. For example, the ability of the antisense oligonucleotides to inhibit KCNT1 expression and/or whole cell current can be assessed in in vitro assays to confirm that the antisense oligonucleotides are suitable for use in treating a disease or condition associated with a gain-of-function mutation in KCNT1 and/or excessive neuronal excitability. Mouse models can be used to not only assess the ability of the antisense oligonucleotides to inhibit KCNT1 expression or whole cell current, but to also ameliorate symptoms associated with gain-of-function KCNT1 mutations and/or excessive neuronal excitability.

In one example, cells such as mammalian cells (e.g. CHO cells) that are transfected with KCNT1 and express this gene are also transfected with an antisense oligonucleotide of the present disclosure. Typically, the KCNT1 contains a gain-of-function mutation. In another example, a human neuronal cell line (e.g. SH-SY5Y) that naturally expresses native wild type KCNT1 is used. Optionally, the genome of this cell is edited so as to contain a gain-of-function mutation, such that the resulting KCNT1 is a disease causing variant. The levels of KCNT1 mRNA can be assessed using qRT-PCR or Northern blot as is well known in the art. The level of expression of protein from KCNT1 can be assessed by Western blot on total cell lysates or fractions as described in Rizzo et al. (Mol Cell Neurosci. (2016) 72:54-63). Residual function of the KCNT1-encoded channels can also be assessed using electrophysiology or ion flux assay.

In a particular examples, the activity of the antisense oligonucleotides of the present disclosure are assessed and confirmed using stem cell modelling (for review, see e.g. Tidball and Parent (2016) Stem Cells 34:27-33; Parent and Anderson (2015) Nature Neuroscience 18:360-366). For example, human induced pluripotent stem cells (iPSCs) can be produced from somatic cells (e.g. dermal fibroblasts or blood-derived hematopoietic cells) derived from a patient with a KCNT1 gain-of-function mutation and presenting with an associated disease or condition (e.g. EIMFS, ADNFLE or West syndrome). Optionally, genome editing can be used to revert the gain-of-function mutation to wild-type to produce an isogenic control cell line (Gaj et al. (2013) Trends Biotechnol 31, 397-405), which can also be used to determine desirable wild-type levels of activity for subsequent assessment and comparison of oligonucleotides. Alternatively, genome editing can be used to introduce a gain-of-function mutation into the KCNT1 gene of wild-type, control iPSCs (e.g. a reference iPSC line). The iPSCs containing the gain-of-function mutation, and optionally the isogenic control, can then be differentiated into neurons, including excitatory neurons, using known techniques (see e.g. Kim et al. (2014) Front Cell Neurosci 8:109; Zhang et al. 2013, Chambers et al. (2009) Nat Biotechnol 27, 275-280). The effect of the antisense oligonucleotides of the present invention on KCNT1 expression (as assessed by KCNT1 mRNA or protein levels) and/or activity (as assessed by ion flux assay and/or electrophysiology, e.g. using the whole cell patch clamp technique, the single electrode voltage clamp technique or the two-electrode voltage clamp (TEVC) technique) can then be assessed following exposure of the iPSCs to the antisense oligonucleotides of the present invention.

The levels of KCNT1 expression (mRNA or protein) or whole cell current observed when cells expressing KCNT1 are exposed to an antisense oligonucleotide of the present disclosure are compared to the respective levels observed when cells expressing KCNT1 are exposed with a negative control antisense oligonucleotide, so as to determine the level of inhibition resulting from the antisense oligonucleotide of the present disclosure. Typically, expression levels of KCNT1 or whole cell current levels are reduced by at least or about 20%, 25%, 30%, 35%, 40%, 45%, 50%, 55%, 60%, 65%, 70%, 75%, 80%, 85%, 90% or more. Accordingly, the antisense oligonucleotides of the present disclosure can be used for treating a disease or condition associated with a gain-of-function mutation in KCNT1.

Mouse models can also be used to assess and confirm the activity of the antisense oligonucleotides of the present disclosure. For example, knock-in or transgenic mouse models can be generated using KCNT1 genes containing a gain-of-function mutation in a similar manner to that described for SCN1A and SCN2A knock-in and transgenic mouse models (see e.g. Kearney et al. (2001) Neuroscience 102, 307-317; Ogiwara et al. (2007) J Neurosci 27:5903-5914; Yu et al. (2006) Nat Neurosci 9:1142-1149). In particular examples, a KCNT1 gene that matches the particular antisense oligonucleotide (e.g. an allele-specific oligonucleotide) is used to produce the knock-in or transgenic mouse. The gain-of-function KCNT1 knock-in or transgenic mice may present with a phenotype similar to EIMFS, ADNFLE and/or West syndrome, including, for example, increased neuronal activity, spontaneous seizures, and heterogeneous focal seizure activity on electroencephalogram (EEG). In other examples, SCN1A and SCN2A knock-in and transgenic mouse models may be used for models exhibiting excessive neuronal excitability. The ability of the antisense oligonucleotides of the present invention to inhibit expression of KCNT1 in these mice and to ameliorate any symptoms associated with the gain-of-function KCNT1 mutations and/or excessive neuronal excitability in the mice, can then be assessed.

For example, the levels of KCNT1 mRNA and/or protein can be assessed following administration of an antisense oligonucleotide of the present disclosure or a negative control antisense oligonucleotide to the mice. In a particular example, KCNT1 mRNA and/or protein levels in the brain, and in particular the neurons, are assessed. The levels of KCNT1 expression following administration of an antisense oligonucleotide of the present disclosure are compared to the respective levels observed when a negative control antisense oligonucleotide is administered, so as to determine the level of inhibition resulting from the antisense oligonucleotide of the present disclosure. Typically, expression levels of KCNT1 in the mice (e.g. in the brains of the mice) are reduced by at least or about 20%, 25%, 30%, 35%, 40%, 45%, 50%, 55%, 60%, 65%, 70%, 75%, 80%, 85%, 90% or more.

In another example, the functional effect of administration of an antisense oligonucleotide of the present disclosure is assessed. For example, the number, severity and/or type of seizures can be assessed visually and/or by EEG. Neuronal excitability can also be assessed, such as by excising brain slices from mice administered an antisense oligonucleotide of the present disclosure or a negative control antisense oligonucleotide and assessing whole cell current (e.g. using the whole cell patch clamp technique). Similar neuronal excitability analyses can be performed using neurons isolated from the mice and then cultured. Additionally, mouse behaviour, including gait characteristics, can be assessed to determine the functional effect of administration of an antisense oligonucleotide of the present disclosure.

### Compositions

The present disclosure provides compositions comprising the antisense oligonucleotides described above and herein. In particular examples, provided are pharmaceutical compositions comprising the antisense oligonucleotides and a pharmaceutically acceptable carrier. The compositions can also comprise additional ingredients such as carriers, diluents, stabilizers and excipients.

The carriers, diluents, stabilizers and excipients can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The antisense oligonucleotides may also be formulated in compositions with liposomes, nanoparticles, microparticles, microspheres, lipid particles, vesicles, and the like, for the introduction of the antisense oligonucleotides of the present disclosure into cells.

Compositions comprising the antisense oligonucleotides encompass compositions comprising any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure also provides pharmaceutically acceptable salts of the antisense oligonucleotides described herein and other bio equivalents. Suitable pharmaceutically acceptable salts include, but are not limited to, sodium and potassium salts.

### Methods of Treating a Disease or Condition Associated with Excessive Neuronal Excitability and/or Gain-Of-Function Mutation in KCNT1

The antisense oligonucleotides described above and herein can be used to treat a disease or condition associated with excessive neuronal excitability and/or a gain-of-function mutation in KCNT1. Exemplary diseases or conditions include EIMFS, ADNFLE and West syndrome. Other exemplary diseases include infantile spasms, epileptic encephalopathy, focal epilepsy, Ohtahara syndrome, developmental epileptic encephalopathy and Lennox Gastaut syndrome. Accordingly, the antisense oligonucleotides and compositions thereof can be administered to a subject with EIMFS, ADNFLE, West syndrome, infantile spasms, epileptic encephalopathy, focal epilepsy, Ohtahara syndrome, developmental epileptic encephalopathy, Lennox Gastaut syndrome or another disease or condition associated with excessive neuronal excitability and/or a gain-of-function mutation in KCNT1.

In some examples, the subject presenting with a disease or condition that may be associated with a gain-of-function mutation in KCNT1 is genotyped to confirm the presence of a known gain-of-function mutation in KCNT1 prior to administration of the antisense oligonucleotides and compositions thereof. For example, whole exome sequencing can be performed on the subject. Gain-of-function mutations associated with EIMFS may include, but are not limited to, V271F, G288S, R428Q, R474Q, R474H, R474C, I760M, A934T and P924L. Gain-of-function mutations associated with ADNFLE may include, but are not limited to, M896I, R398Q, Y796H and R928C. Gain-of-function mutations associated with West syndrome may include, but are not limited to, G652V and R474H. In other examples, the subject is first genotyped to identify the presence of a mutation in KCNT1 and this mutation is then confirmed to be a gain-of-function mutation using standard in vitro assays, such as those described in Milligan et al. (2015) Ann Neurol. 75(4): 581-590. Typically, the presence of a gain-of-function mutation is confirmed when the expression of the mutated KCNT1 allele results an increase in whole cell current compared to the whole cell current resulting from expression of wild-type KCNT1 as assessed using whole-cell electrophysiology (such as described in Milligan et al. (2015) Ann Neurol. 75(4): 581-590; Barcia et al. (2012) Nat Genet. 44(11): 1255-1259; Mikati et al. (2015) Ann Neurol. 78(6): 995-999; or Rizzo et al. Mol Cell Neurosci. (2016) 72:54-63). This increase of whole cell current can be, for example, an increase of at least or about 50%, 100%, 150%, 200%, 250%, 300%, 350%, 400% or more. The subject can then be confirmed to have a disease or condition associated with a gain-of-function mutation in KCNT1.

In instances where the antisense oligonucleotides are allele-specific antisense oligonucleotides, the subject is also genotyped to determine which allele the gain-of-function mutation is present on, i.e. which allele-specific SNP the gain-of-function mutation is associated with. Identification of an allele-specific SNP that is associated with the gain-of-function mutation and thus present on the mutant allele but not present on the wild-type KCNT1 allele informs which of the allele-specific antisense oligonucleotides of the present disclosure should be used for treatment. Allele-specific SNPs can include those described above, such as, for example, SNP 9:138594266 A/AC (rs5901089) at nt 5236, SNP 9:138662309 A/G (rs10776844) at nt 73279, SNP 9:138669261 G/A (rs914428 at nt 80231, SNP 9:138662661 G/A (rs10858172) at nt 73631, SNP 9:138642912 C/A (rs10122976) at nt 53882, SNP 9:138644203 T/C (rs10735239) at nt 55173, SNP 9:138628353 C/T (rs7350168) at nt 39323, and SNP 9:138680901 T/C (rs10858173) at nt 91871 of SEQ ID NO:1. Based on such genotyping, the skilled person can select the allele-specific antisense oligonucleotide of the present invention that is complementary to the region spanning the SNP identified as being associated with the gain-of-function mutation.

The antisense oligonucleotides can also be used therapeutically for conditions associated with excessive neuronal excitability where the excessive neuronal excitability is not necessarily the result of a gain-of-function mutation in KCNT1. Even in instances where the disease is not the result of increased KCNT1 expression and/or activity, inhibition of KCNT1 expression and/or activity can nonetheless result in a reduction in neuronal excitability, thereby providing a therapeutic effect. Thus, the antisense oligonucleotides of the present disclosure can be used to treat, for example, a subject with EIMFS, ADNFLE, West syndrome, infantile spasms, epileptic encephalopathy, focal epilepsy, Ohtahara syndrome, developmental epileptic encephalopathy, Lennox Gastaut syndrome, regardless of whether or not the disease is associated with a gain-of-function mutation in KCNT1.

The precise amount or dose of the antisense oligonucleotide administered to the subject depends on, for example, the efficacy of the antisense oligonucleotide, the presence of other moieties (e.g. CCPs), the route of administration, the number of dosages administered, and other considerations, such as the weight, age and general state of the subject. Particular dosages and administration protocols can be empirically determined or extrapolated from, for example, studies in animal models or previous studies in humans, or may be otherwise determined by those skilled in the art using standard procedures.

The antisense oligonucleotides can be administered by any method and route understood to be suitable by a skilled artisan. Typically, the antisense oligonucleotides are administered parenterally, such as by subcutaneous administration, intravenous administration, intramuscular administration, intraarterial administration, intraperitoneal administration, or intracranial administration, e.g., intrathecal or intracerebroventricular administration. In other embodiments, the antisense oligonucleotides are delivered intranasally. Administration of the antisense oligonucleotides in the methods described herein preferably results in delivery of the antisense oligonucleotides to the central nervous system. In particular embodiments, the antisense oligonucleotides are administered intrathecally or by intracerebroventricular administration. The methods of the present invention can involve any combination of any two or more routes.

The antisense oligonucleotides can be administered to a subject one time or more than one time, including 2, 3, 4, 5 or more times. Typically, the antisense oligonucleotides are administered as needed while symptoms (e.g. seizures) remain. Where the antisense oligonucleotides are administered more than one time, the time between dosage administration can be, for example, 1, 2, 3, 4, 5, 6, 7, 8, 9, 10, 11, 12 or more weeks, or 1, 2, 3, 4, 5, 6, 7, 8, 9, 10, 11, 12 or more months. Selecting an optimal protocol is well within the level of skill of the skilled artisan and may depend on, for example, the half-life of the antisense oligonucleotide and the severity of the condition. In a particular embodiment, the antisense oligonucleotides are administered about every 3 months.

The antisense oligonucleotides, if desired, can be presented in a package, in a kit or dispenser device, such as a syringe with a needle, or a vial and a syringe with a needle, which can contain one or more unit dosage forms. The kit or dispenser device can be accompanied by instructions for administration.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1

Two locked nucleic acid (LNA) oligonucleotides specific for the mRNA of mouse KCNT1 were ordered from Exiqon (Qiagen). The sequences of the two oligonucleotides were as follows:

(SEQ ID NO: 4)
mKcnt1_LNA 5: 5′ TGAGAAAGTCACGATG 3′
(SEQ ID NO: 5)
mKcnt1_LNA 6: 5′ GAGGTGGCATAAAGTC 3′

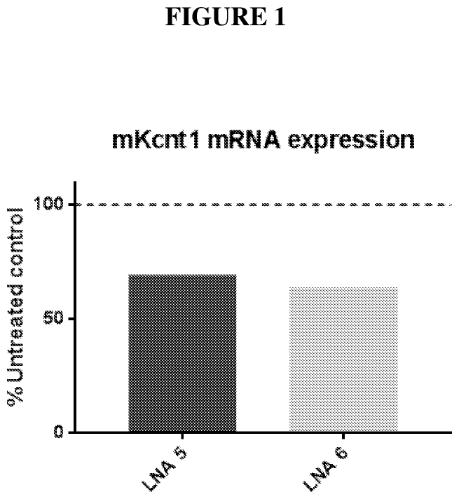

Two L of LNA oligonucleotides at a concentration of 5 g/L (5 nanomoles) were injected into wild-type BL/6 mice at P1 in the right lateral ventricle. Brains were then harvested 14 days after injection and RNA was isolated from the right hemisphere using a standard trizol-based extraction protocol. qPCR was performed using primers specific for KCNT1 mRNA, as well as those specific for RPL32 as a house gene. KCNT1 mRNA expression levels were determined and normalised to RPL32 expression, then expressed as a percentage of the KCNT1 mRNA expression levels in untreated wild-type mice. As shown in FIG. 1, administration of either LNA 5 or LNA 6 effectively inhibited KCNT1 mRNA expression levels in the brains of mice.

1. An antisense oligonucleotide comprising a sequence of nucleobases that is complementary to a target region in KCNT1.

2. The antisense oligonucleotide of claim 1, wherein the target region is within the KCNT1 sequence set forth in SEQ ID NO:1 or a variant thereof having at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereto.

3. The antisense oligonucleotide of claim 1 or claim 2, wherein the antisense oligonucleotide hybridizes to the pre-mRNA and/or mRNA of KCNT1.

4. The antisense oligonucleotide of any one of claims 1-3, wherein the target region is within or spans all or a part of an exon, intron, an intron/exon junction, a 3′-untranslated region (UTR), a 5′-UTR, the translation initiation site and/or the translation termination site.

5. The antisense oligonucleotide of any one of claims 1-4, wherein the antisense oligonucleotide is an allele-specific oligonucleotide.

6. The antisense oligonucleotide of any one claims 1-5, wherein the target region spans a nucleotide selected from among nucleotide 5236, 39323, 53882, 55173, 73279, 73631, 80231 or 91871 of SEQ ID NO:1.

7. The antisense oligonucleotide of claim 6, wherein the antisense oligonucleotide specifically hybridizes to the pre-mRNA of a KCNT1 allele containing nucleotide(s) AC at position 5236, T at position 39323, C at position 55173, A at position 53882, G at position 73279, A at position 73631, A at position 80231, or C at position 91871.

8. The antisense oligonucleotide of any one claims 1-7, wherein the antisense oligonucleotide is 10 to 80, 10 to 60, 10 to 50, 10 to 40, 10 to 30 or 15 to 25 nucleobases in length.

9. The antisense oligonucleotide of any one claims 1-8, wherein the antisense oligonucleotide is at least 70%, 80%, 85%, 86%, 87%, 88%, 89%, 90%, 91%, 92%, 93%, 94%, 95%, 96%, 97%, 98%, or 99% complementary to the target region.

10. The antisense oligonucleotide of any one claims 1-9, wherein the antisense oligonucleotide comprises least 8, 9, 10, 11, 12, 13, 14, 15, 16, 17, 18, 19 or 20 contiguous nucleobases that are 100% complementary to the target region.

11. The antisense oligonucleotide of any one of claims 1-10, wherein the antisense oligonucleotide comprises at least one modification.

12. The antisense oligonucleotide of claim 11, wherein the modification is a nucleobase modification, a modification of the oligonucleotide backbone or a modification of a ribose sugar.

13. The antisense oligonucleotide of claim 12, wherein antisense oligonucleotide comprises a modified sugar selected from among a 2′-O-methyl (2OMe), 2′-O-methoxy-ethyl (MOE), locked nucleic acids (LNA), 2′-fluoro or S-constrained-ethyl (cEt).

14. The antisense oligonucleotide of claim 11 or claim 12, wherein the backbone of the antisense oligonucleotide comprises phosphorothioates.

15. The antisense oligonucleotide of any one of claims 1-14, wherein the antisense oligonucleotide activates RNase H.

16. A composition comprising the antisense oligonucleotide of any one of claims 1-15.

17. A method for treating a disease or condition associated with a gain-of-function mutation in KCNT1 in a subject, comprising administering to the subject the antisense oligonucleotide of any one of claims 1-15 or composition of claim 16.

18. The method of claim 17, wherein the disease or condition is selected from among epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome.

19. The method of claim 17 or claim 18, wherein the subject is confirmed as having a KCNT1 allele containing a gain-of-function mutation.

20. The method of claim 19, wherein the gain-of-function mutation is selected from among V271F, G288S, R398Q, R428Q, R474Q, R474H, R474C, G652V, I760M, Y796H, M896I, P924L, R928C and A934T.

21. The method of any one of claims 17-20, comprising administering to the subject an allele-specific antisense oligonucleotide of any one of claims 5-15.

22. The method of claim 21, wherein the subject has been genotyped to identify an allele-SNP that is associated with the gain-of-function mutation.

23. The method of claim 22, wherein the allele-specific SNP is selected from among SNP 9:138594266 A/AC (rs5901089) at nucleotide (nt) 5236 of SEQ ID NO:1, SNP 9:138662309 A/G (rs10776844) at nt 73279, SNP 9:138669261 G/A (rs914428 at nt 80231, SNP 9:138662661 G/A (rs10858172) at nt 73631, SNP 9:138642912 C/A (rs10122976) at nt 53882, SNP 9:138644203 T/C (rs10735239) at nt 55173, SNP 9:138628353 C/T (rs7350168) at nt 39323, and SNP 9:138680901 T/C (rs10858173) at nt 91871.

24. A method of treating a disease or condition selected from among epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome, comprising administering to the subject the antisense oligonucleotide of any one of claims 1-15 or composition of claim 16.

25. The method of any one of claims 17-24, wherein the antisense oligonucleotide or composition is administered to the subject by parenteral administration or intranasal administration.

26. The method of claim 25, wherein the parenteral administration is selected from among subcutaneous administration, intravenous administration, intramuscular administration, intraarterial administration, intraperitoneal administration, or intracranial administration.

27. The method of claim 26, wherein intracranial administration is intrathecal or intracerebroventricular administration.

28. The method of any one of claims 17-27, wherein the antisense oligonucleotide or composition is administered to the subject about every 1, 2, 3, 4, 5, 6, 7, 8, 9, 10, 11, 12 or more months.

29. The method of any one of claims 17-28, wherein the antisense oligonucleotide or composition is administered to the subject about every 3 months.

30. Use of the antisense oligonucleotide of any one of claims 1-15 or composition of claim 16 for the preparation of a medicament for treating a disease or condition associated with a gain-of-function mutation in KCNT1.

31. Use of the antisense oligonucleotide of any one of claims 1-15 or composition of claim 16 for the preparation of a medicament for treating a disease or condition selected from among epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental epileptic encephalopathy, and Lennox Gastaut syndrome.
